(19) United States
(12) Patent Application Publication
(10) Pub. No.: US 2025/0295137 A1
(43) Pub. Date: Sep. 25, 2025

(54) PROBIOTIC COMPOSITIONS FOR AQUACULTURE

(71) Applicant: BiomEdit, LLC, Fishers, IN (US)

(72) Inventors: Arvind Kumar, Fishers, IN (US); Dharanesh Mahimapura Gangaiah, Fishers, IN (US); Hannah Bekebrede, Fishers, IN (US)

(21) Appl. No.: 18/692,412

(22) PCT Filed: Sep. 19, 2022
(86) PCT No.: PCT/US2022/043996

§ 371 Date: Mar. 15, 2024

Related U.S. Application Data
(60) Provisional application No. 63/245,791, filed on Sep. 17, 2021.
(60) Provisional application No. 63/293,168, filed on Dec. 23, 2021.

(51) Int. Cl. A23K 10/16; A23K 20/163; A23K 20/174; A23K 20/20; A23K 50/75; A23K 50/80; C12N 1/20; C12R 1/07; C12R 1/225

(52) U.S. Cl. CPC A23K 10/16; A23K 20/163; A23K 20/174; A23K 20/20; A23K 50/75; A23K 50/80; C12N 1/20; C12R 2001/07; C12R 2001/225

(57) ABSTRACT

Disclosed are probiotic compositions and methods for improving animal health and animal production. The probiotic compositions include two or more novel Lactilactobacillus strains which are capable of colonizing the gastrointestinal tract to improve the health of an animal. A probiotic composition includes Lactilactobacillus curvatus and a second Lactilactobacillus strain. The second Lactilactobacillus strain can include a second strain of Lactilactobacillus curvatus, Lactilactobacillus sakei, Lactilactobacillus fuchuensis, and combinations thereof, and a carrier suitable for animal administration. Also disclosed are Bacillus strains.

### CROSS-REFERENCE TO RELATED APPLICATIONS

This application is a national stage application which claims priority from PCT Application No. PCT/US2022/043996 filed Sep. 19, 2022, which in turn claims priority to U.S. provisional application Ser. No. 63/245,791 filed Sep. 17, 2021 and 63/293,168 filed Dec. 23, 2021, all of which are incorporated herein by reference in their entireties.

### SEQUENCE LISTING XML

The instant application contains a Sequence Listing encoded in XML format which has been filed electronically by EFS-web and is hereby incorporated by reference in its entirety. Said XML format Sequence Listing, created on Nov. 8, 2024, is named “2950-5 PCT_US_ST26.xml” and is 35,960,286 bytes in size.

### FIELD

The present invention relates to probiotic compositions and methods for improving animal health. The probiotic compositions include one or more isolated strains of Lactobacillus species which colonizes the gastrointestinal tract to improve the health and production performance of an animal.

### BACKGROUND

Direct fed microbials (DFMs), often also called probiotics, are microorganisms which colonize the gastrointestinal tract of an animal and provide some beneficial effect to that animal. The microorganisms can be bacterial species, for example, those from the genera Bacillus, Lactobacillus, Lactococcus, and Enterococcus. The microorganisms can also be yeast or even molds. The microorganisms can be provided to an animal orally or mucosally or, in the case of birds, provided to a fertilized egg, i.e., in ovo.

The beneficial activity provided by a DFM can be through the synthesis and secretion of vitamins or other nutritional molecules needed for a healthy metabolism of the host animal. A DFM can also protect the host animal from disease, disorders, or clinical symptoms caused by pathogenic microorganisms or other agents. For example, the DFM may naturally produce factors having inhibitory or cytotoxic activity against certain species of pathogens, such as deleterious or disease-causing bacteria.

Probiotics and DFMs provide an attractive alternative or addition to the use and application of antibiotics in animals. Antibiotics can promote resistant or less sensitive bacteria and can ultimately end up in feed products or foods consumed by other animals or humans. There is a need in the art for probiotic compositions and methods that provide improved delivery of beneficial molecules to the gastrointestinal tract of an animal and thus improve animal health.

The citation of references herein shall not be construed as an admission that such is prior art to the present disclosure.

### SUMMARY

An aspect of the disclosure is a probiotic composition including Lactilactobacillus species. The Lactilactobacillus species includes a first Lactilactobacillus curvatus strain and a second Lactilictobacillus strain, and a carrier suitable for animal administration.

In an embodiment, the second Lactilactobacillus strain comprises at least one of a second Lactilactobacillus curvatus strain, Lactilactobacillus sakei, Lactilactobacillus fuchuensis and combinations thereof.

In embodiments, the Lactilactobacillus strains of the disclosure include any combination of any two, three, four or more of the following isolated Lactilactobacillus strains and any combination of any two, three, four or more of the following strains having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the genomic sequence of any of the SEQ ID NOS: 1-19. Further Lactilactobacillus strains of this disclosure include any combination of any two, three, four or more strains having at least 80% identity, 90% identity, 95% identity, 97% identity, 98% identity, 99% identity in sequence to the coding sequences of SEQ ID NOS: 1-19 or a portion or portions thereof. The whole genome nucleic acid sequences for the Lactilactobacillus strains of Table 1 are provided in SEQ ID NOS: 1-19.

TABLE 1
Accession numbers of Lactilactobacillus strains of the disclosure.

| | Isolated | Bioproject | SEQ ID |
|---|---|---|---|
| Organism | strains | accession | NO: |
| Lactilactobacillus | ELA204093 | PRJNA762592 | 1 |
| curvatus | | | |
| Lactilactobacillus | ELA204100 | PRJNA762592 | 2 |
| curvatus | | | |
| Lactilactobacillus | ELA214388 | PRJNA762592 | 3 |
| curvatus | | | |
| Lactilactobacillus | ELA214391 | PRJNA762593 | 4 |
| sakei | | | |
| Lactilactobacillus | ELA214002 | PRJNA762592 | 5 |
| curvatus | | | |
| Lactilactobacillus | ELA204023 | PRJNA762592 | 6 |
| curvatus | | | |
| Lactilactobacillus | ELA204029 | PRJNA762592 | 7 |
| curvatus | | | |
| Lactilactobacillus | ELA204033 | PRJNA762592 | 8 |
| curvatus | | | |
| Lactilactobacillus | ELA214059 | PRJNA762592 | 9 |
| curvatus | | | |
| Lactilactobacillus | ELA214060 | PRJNA762592 | 10 |
| curvatus | | | |
| Lactilactobacillus | ELA214061 | PRJNA762592 | 11 |
| curvatus | | | |
| Lactilactobacillus | ELA214062 | PRJNA762592 | 12 |
| curvatus | | | |
| Lactilactobacillus | ELA204092 | PRJNA762592 | 13 |
| curvatus | | | |
| Lactilactobacillus | ELA204096 | PRJNA762592 | 14 |
| curvatus | | | |
| Lactilactobacillus | ELA204098 | PRJNA762592 | 15 |
| curvatus | | | |
| Lactilactobacillus | ELA214117 | PRJNA762592 | 16 |
| curvatus | | | |
| Lactilactobacillus | ELA214068 | PRJNA780402 | 17 |
| fuchuensis | | | |
| Lactilactobacillus | ELA214064 | PRJNA762593 | 18 |
| sakei | | | |
| Lactilactobacillus | ELA214065 | PRJNA762593 | 19 |
| sakei | | | |

In an embodiment, the first Lactilactobacillus curvatus strain includes any of the following strains:
Lactilactobacillus curvatus ELA204093 corresponding to ATCC deposit PTA-127116, Lactilactobacillus curvatus ELA204100 corresponding to ATCC deposit PTA-127117 and Lactilactobacillus curvatus ELA214388 corresponding to ATCC deposit PTA-127118, and combinations thereof, or
a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NOS: 1-3, and combinations thereof.

In an embodiment the second Lactilactobacillus strain comprises any of the following strains which is different than the first Lactilactobacillus curvatus strain:
Lactilactobacillus curvatus ELA204093 corresponding to ATCC deposit PTA-127116, Lactilactobacillus curvatus ELA204100 corresponding to ATCC deposit PTA-127117, Lactilactobacillus curvatus ELA214388 corresponding to ATCC deposit PTA-127118, Lactilactobacillus sakei ELA214391 corresponding to ATCC deposit PTA-127119 and combinations thereof, or
a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NOS: 1-4, and combinations thereof.

In an embodiment the first Lactilactobacillus curvatus strain includes any of the following strains:
at least one of Lactilactobacillus curvatus ELA214002, Lactilactobacillus curvatus ELA204023, Lactilactobacillus curvatus ELA204029, Lactilactobacillus curvatus ELA204033, Lactilactobacillus curvatus ELA214059, Lactilactobacillus curvatus ELA214060, Lactilactobacillus curvatus ELA214061, Lactilactobacillus curvatus ELA214062, Lactilactobacillus curvatus ELA204092, Lactilactobacillus curvatus ELA204096, Lactilactobacillus curvatus ELA204098, Lactilactobacillus curvatus ELA214117; or
at least one Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to any of sequences of SEQ ID NO: 5-16; and combinations thereof.

In an embodiment, a) the first Lactilactobacillus curvatus strain includes any of the following strains:
at least one of Lactilactobacillus curvatus ELA214002, Lactilactobacillus curvatus ELA204023, Lactilactobacillus curvatus ELA204029, Lactilactobacillus curvatus ELA204033, Lactilactobacillus curvatus ELA214059, Lactilactobacillus curvatus ELA214060, Lactilactobacillus curvatus ELA214061, Lactilactobacillus curvatus ELA214062, Lactilactobacillus curvatus ELA204092, Lactilactobacillus curvatus ELA204096, Lactilactobacillus curvatus ELA204098, Lactilactobacillus curvatus ELA214117; and combinations thereof; or
at least one Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to any of sequences of SEQ ID NO: 5-16; and combinations thereof; and
b) the second Lactilactobacillus strain comprises any of the following strains which is different than the first Lactilactobacillus curvatus strain:
at least one of Lactilactobacillus curvatus ELA214002, Lactilactobacillus curvatus ELA204023, Lactilactobacillus curvatus ELA204029, Lactilactobacillus curvatus ELA204033, Lactilactobacillus curvatus ELA214059, Lactilactobacillus curvatus ELA214060, Lactilactobacillus curvatus ELA214061, Lactilactobacillus curvatus ELA214062, Lactilactobacillus curvatus ELA204092, Lactilactobacillus curvatus ELA204096, Lactilactobacillus curvatus ELA204098, Lactilactobacillus curvatus ELA214117, Lactilactobacillus fuchuensis ELA214068, Lactilactobacillus sakei ELA214064, Lactilactobacillus sakei ELA214065; or
at least one Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to any of sequences of SEQ ID NO: 5-19; and combinations thereof.

In an embodiment, a) the first Lactilactobacillus curvatus strain includes any of the following strains:
Lactilactobacillus curvatus ELA204093 corresponding to ATCC deposit PTA-127116, Lactilactobacillus curvatus ELA204100 corresponding to ATCC deposit PTA-127117 and Lactilactobacillus curvatus ELA214388 corresponding to ATCC deposit PTA-127118, and combinations thereof, or
a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NOS: 1-3, and combinations thereof; and
b) wherein the first Lactilactobacillus curvatus strain further includes at least one of Lactilactobacillus curvatus ELA214002, Lactilactobacillus curvatus ELA204023, Lactilactobacillus curvatus ELA204029, Lactilactobacillus curvatus ELA204033, Lactilactobacillus curvatus ELA214059, Lactilactobacillus curvatus ELA214060, Lactilactobacillus curvatus ELA214061, Lactilactobacillus curvatus ELA214062, Lactilactobacillus curvatus ELA204092, Lactilactobacillus curvatus ELA204096, Lactilactobacillus curvatus ELA204098, Lactilactobacillus curvatus ELA214117, and combinations thereof; or
at least one Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to any of sequences of SEQ ID NO: 5-16; and combinations thereof.

In an embodiment, a) the first Lactilactobacillus curvatus strain includes any of the following strains:
Lactilactobacillus curvatus ELA204093 corresponding to ATCC deposit PTA-127116, Lactilactobacillus curvatus ELA204100 corresponding to ATCC deposit PTA-127117 and Lactilactobacillus curvatus ELA214388 corresponding to ATCC deposit PTA-127118, and combinations thereof, or
a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NOS: 1-3, and combinations thereof; and
b) the second Lactilactobacillus strain comprises any of the following strains which is different than the first Lactilactobacillus curvatus strain:
at least one of Lactilactobacillus curvatus ELA214002, Lactilactobacillus curvatus ELA204023, Lactilactobacillus curvatus ELA204029, Lactilactobacillus curvatus ELA204033, Lactilactobacillus curvatus ELA214059, Lactilactobacillus curvatus ELA214060, Lactilactobacillus curvatus ELA214061, Lactilactobacillus curvatus ELA214062, Lactilactobacillus curvatus ELA204092, Lactilactobacillus curvatus ELA204096, Lactilactobacillus curvatus ELA204098, Lactilactobacillus curvatus ELA214117, Lactilactobacillus fuchuensis ELA214068, Lactilactobacillus sakei ELA214064, Lactilactobacillus sakei ELA214065; and combinations thereof; or
at least one Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to any of sequences of SEQ ID NO: 5-19; and combinations thereof.

An embodiment is a probiotic composition including isolated Lactilactobacillus species, and a carrier suitable for animal administration. The isolated Lactilactobacillus species comprises at least two of the following:
ELA204093 corresponding to ATCC deposit PTA-127116 (PTA-16) or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 1;
ELA204100 corresponding to ATCC deposit PTA-127117 (PTA-17) or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 2;
ELA214388 corresponding to ATCC deposit PTA-127118 (PTA-18) or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 3; or
ELA214391 corresponding to ATCC deposit PTA-127119 (PTA 19) or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 4.

In an embodiment a probiotic composition includes a first isolated Lactilactobacillus curvatus strain and a second isolated Lactilactobacillus strain; and a carrier suitable for animal administration;
wherein the composition reduces or inhibits the colonization of an animal by a pathogenic bacterium or provides improved growth performance in an animal when an effective amount is administered to an animal, as compared to an animal not administered the composition; and
wherein the first isolated Lactilactobacillus curvatus strain comprises a nucleic acid sequence having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 1 and wherein the second isolated Lactilactobacillus strain comprises a nucleic acid sequence having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 2; or
wherein the first isolated Lactilactobacillus curvatus strain comprises a nucleic acid sequence having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 3 and wherein the second isolated Lactilactobacillus strain comprises a nucleic acid sequence having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 4.

In an embodiment a probiotic composition includes a first isolated Lactilactobacillus curvatus strain and a second isolated Lactilactobacillus strain; and a carrier suitable for animal administration;
wherein the composition reduces or inhibits the colonization of an animal by a pathogenic bacterium or provides improved growth performance in an animal when an effective amount is administered to an animal, as compared to an animal not administered the composition; and
wherein the first isolated Lactilactobacillus curvatus strain comprises a nucleic acid sequence having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 1, and wherein the second isolated Lactilactobacillus strain comprises a nucleic acid sequence having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 3; or
wherein the first isolated Lactilactobacillus curvatus strain comprises a nucleic acid sequence having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 2; and wherein the second isolated Lactilactobacillus strain comprises a nucleic acid sequence having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 4.

An embodiment of a composition includes:
ELA204093 corresponding to ATCC deposit PTA116 or a first Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 1; or
ELA204100 corresponding to ATCC deposit PTA-117 or a second Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 2; or
ELA214388 corresponding to ATCC deposit PTA-118 or a third Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 3; or
ELA214391 corresponding to ATCC deposit PTA-119 or a fourth Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 4;
wherein the composition includes at least one of the following combinations:
ELA204093 or the first Lactilactobacillus strain, and ELA204100 or the second Lactilactobacillus strain;
ELA214388 or the third Lactilactobacillus strain, and ELA214391 or the fourth Lactilactobacillus strain;
ELA204093 or the first Lactilactobacillus strain, and ELA214388 or the third Lactilactobacillus strain;
ELA204093 or the first Lactilactobacillus strain, and ELA214391 or the fourth Lactilactobacillus strain;
ELA204100 or the second Lactilactobacillus strain, and ELA214388 or the third Lactilactobacillus strain;
ELA204100 or the second Lactilactobacillus strain, and ELA214391 or the fourth Lactilactobacillus strain; and combinations thereof.

In some embodiments, a composition of the disclosure includes a first isolated Lactilactobacillus curvatus strain including ELA204093 and a second isolated Lactilactobacillus strain including ELA204100. In some embodiments, a composition of the disclosure includes a first isolated Lactilactobacillus curvatus strain including ELA214388 and a second isolated Lactilactobacillus strain including ELA214391. In some embodiments, a composition of the disclosure includes a first isolated Lactilactobacillus curvatus strain including ELA204093 and a second isolated Lactilactobacillus strain including ELA214391. In some embodiments, a composition of the disclosure includes a first isolated Lactilactobacillus curvatus strain including ELA204093 and a second isolated Lactilactobacillus strain including ELA214388. In some embodiments, a composition of the disclosure includes a first isolated Lactilactobacillus curvatus strain including ELA204100 and a second isolated Lactilactobacillus strain including ELA2214388. In some embodiments, a composition of the disclosure includes a first isolated Lactilactobacillus curvatus strain including ELA204100 and a second isolated Lactilactobacillus strain including ELA214391.

In embodiments, the Lactilactobacillus strains are selected from ELA204093 corresponding to ATCC deposit PTA-127116 or a Lactilactobacillus strain having at least 90% identity, 95% identity, 97% identity, 98% identity, 99% identity in genomic sequence to the sequence of ELA204093 corresponding to ATCC deposit PTA-127116; ELA204100 corresponding to ATCC deposit PTA-127117 or a Lactilactobacillus strain having at least 90% identity, 95% identity, 97% identity, 98% identity, 99% identity in genomic sequence to the sequence of ELA204100 corresponding to ATCC deposit PTA-127117; ELA214388 corresponding to ATCC deposit PTA-127118 or a Lactilactobacillus strain having at least 90% identity, 95% identity, 97% identity, 98% identity, 99% identity in genomic sequence to the sequence of ELA214388 corresponding to ATCC deposit PTA-127118; and ELA214391 corresponding to ATCC deposit PTA-127119 or a Lactilactobacillus strain having at least 90% identity, 95% identity, 97% identity, 98% identity, 99% identity in genomic sequence to the sequence of ELA214391 corresponding to ATCC deposit PTA-127119.

In some embodiments, the disclosure relates to related, homologous or derivative Lactilactobacillus strains having significant genome sequence identity to the genome sequence of any of the following Lactilactobacillus strains: Lactilactobacillus curvatus strain ELA204093 corresponding to ATCC deposit PTA-127116, Lactilactobacillus curvatus strain ELA204100 corresponding to ATCC deposit PTA-127117, Lactilactobacillus curvatus strain ELA214388 corresponding to ATCC deposit PTA-127118, and/or Lactilactobacillus sakei strain ELA214391 corresponding to ATCC deposit PTA-127119. Thus, derivative or similar or nearly genetically identical strains to the Lactilactobacillus strains provided herein are contemplated by and additional embodiments of the disclosure. Lactilactobacillus strains having 80% identity, 85% identity, 90% identity, 95% identity, 97% identity, 98% identity, 99% identity in genomic sequence to a strain provided and deposited in association with this disclosure are contemplated and are embodiments of the disclosure. Such derivative or similar or nearly genetically identical strains must similarly function as probiotics and have activity/capability or function in improving animal health and animal production and performance, including as detailed in the capability and activity or function of the strains and examples hereof.

In an embodiment a ratio of the first Lactilactobacillus curvatus strain and the second Lactilactobacillus strain is 0.75-1.5:1 or 1:0.75-1.5. In one embodiment, a composition is provided comprising strain ELA204093 and ELA204100 in equal amounts or at a ratio of 0.75-1.5:1. In one embodiment, a composition is provided comprising strain ELA214388 and ELA214391 in equal amounts or at a ratio of 0.75-1.5:1.

In an embodiment, the probiotic composition further includes a Bacillus species. The Bacillus species may be Bacillus velezensis, Bacillus subtilis, or a combination thereof.

In an embodiment, the probiotic composition includes at least one of the following bacterial species: B. velezensis ELA006, B. velezensis ELA014, B. subtilis ELA015, B. subtilis ELA017, B. subtilis ELA191105, B. amyloliquefaciens ELA202071, B. amyloliquefaciens ELA191024 and combinations thereof.

In an embodiment, a probiotic composition includes a mixture of the following bacterial species: B. amyloliquefaciens ELA191024, B. subtilis ELA191105 and B. amyloliquefaciens ELA202071.

### Bacillus Subtilis—Strain 105

Bacillus subtilis is a Gram-positive model bacterium which is widely used for industrial production of recombinant proteins such as alpha-amylase, protease, lipase, and other industrial enzymes. Because of the ability of the bacteria to produce large amounts of a target protein, and also to secrete large amounts of a target protein into the culture medium, and the availability of a low-cost downstream production and purification process, over 60% of commercial industrial enzymes are produced in Bacillus subtilis and relative Bacillus species (Schallmey, M.; Singh, A.; Ward, O. P. (2004) 50 (1): 1-17). In contrast to the frequently used recombinant protein expression host Escherichia coli, Bacillus subtilis has no risk of endotoxin contamination and has been certificated as a GRAS (generally regarded as safe) organism by the FDA, which makes it a choice for food-grade and pharmaceutical protein production.

Bacillus subtilis strain ELA191105, also denoted strain 105, corresponds to ATCC deposit PTA-126786. Strain 105 is described and detailed as a genetically modified strain for live delivery or production in U.S. Ser. No. 63/247,271 (filed Sep. 11, 2021), 63/247,273 (filed Sep. 22, 2021) and 63/247,400 (filed Sep. 23, 2021), which applications are incorporated herein by reference.

B. subtilis Strain 105 and Strain Combinations

B. subtilis strain 105 is described as a microbial having beneficial effects, including in combination with one or more Bacillus amyloliquefaciens strain. In some aspects/embodiments, the B. subtilis strain 105 can be combined with one or more isolated Bacillus amyloliquefaciens strain, particularly selected from ELA191024 (corresponding to ATCC deposit PTA-126784), ELA191006 (corresponding to ATCC deposit PTA-127065) and ELA202071 (corresponding to ATCC deposit PTA-127064). These probiotic strain combinations and compositions and methods thereof are described and provided in PCT/US2021/051973 filed Sep. 24, 2021, published as WO2022/067052 Mar. 31, 2022. Priority applications include 63/083,697 filed Sep. 25, 2020 and 63/241,369 filed Sep. 8, 2021. All of the foregoing applications are incorporated herein by reference in their entireties.

In an embodiment the Lactilactobacillus species only has one antimicrobial resistance gene. In an embodiment the Lactilactobacillus species does not have any identifiable antimicrobial resistance genes. In an embodiment, the Lactilactobacillus species only has one gene involved in biogenic amines and toxins. In an embodiment the Lactilactobacillus species does not have any identifiable genes involved in biogenic amines and toxins. In an embodiment the Lactilactobacillus species is sensitive to an antibiotic. In an embodiment the antibiotic comprises at least one of Ampicillin, Vancomycin, Gentamicin, Kanamycin, Streptomycin, Erythromycin, Clindamycin, Tetracycline, Chloramphenicol, or combinations thereof.

In an embodiment the probiotic composition is in the form of a liquid, dry powder, pellets, suspension, or a combination thereof. In an embodiment the composition comprises between about 1×10⁶ and 1×10⁹ CFU/g of the Lactilactobacillus species. In an embodiment the composition comprises at least about 1×10⁶, 1×10⁷ CFU/g, 1×10⁸ CFU/g, or 1×10⁹ CFU/g of the Lactilactobacillus species. In an embodiment the composition includes from about 1×10⁴ to about 1×10¹⁰ viable spores per gram dry weight of the Lactilactobacillus species. In an embodiment the probiotic composition further comprises a prebiotic. In an embodiment the composition further comprises inulin, vitamin D, vitamin C, zinc, N-acetyl-glucosamine, galactooligosaccharides (GOS), lactose, or combinations thereof. In an embodiment the at least one bacterial strain is isolated and inactivated. In an embodiment the at least one bacterial strain is not genetically engineered. In an embodiment the Lactilactobacillus species is obtained from Salmo salar. In one embodiment, the first and the second isolated Lactilactobacillus strain are isolated from salmon and in particular from the intestine of Salmo salar.

In an embodiment the probiotic composition or direct fed microbial of the disclosure is formulated as animal feed, feed additive, food ingredient, water additive, water-mixed additive, consumable solution, consumable spray additive, consumable solid, consumable gel, injection, or combinations thereof. In an embodiment the probiotic composition of the disclosure includes a carrier suitable for animal administration. In an embodiment probiotic composition of the disclosure includes at least one of the following: edible food grade material, mineral mixture, gelatin, cellulose, carbohydrate, starch, glycerin, water, rice hulls, glycol, molasses, calcium carbonate, whey, sucrose, dextrose, soybean oil, vegetable oil, sesame oil, and corn oil, and combinations thereof.

In an embodiment, a method for improving feed efficiency in a fish includes administering the probiotic composition or composition described herein,

In an embodiment, a method for improving disease resistance in a fish includes administering the probiotic composition or composition described herein.

In the method embodiments the fish may be a salmon. The fish may be an Atlantic salmon. In the method embodiments, the composition may improve humoral immune modulation, bacteriocin production, lymphocyte modulation, inhibit aquatic pathogens. The aquatic pathogen may be Aeromonas.

In an embodiment, when combined with prebiotics, the composition may form a synbiotic which improves humoral immune response and weight gain.

In an embodiment, the compositions described herein may be used in the manufacture of a probiotic for fish.

In an embodiment, a fish food product may comprise the composition described herein. The product may comprise about 3-8% w/w lyophilized bacteria. The product may comprise about 0.01-0.2% w/w spray dried spores. The food product may further comprise food-grade excipients. The food product may be in the form of pellets, powder, granules, or a combination thereof.

In an embodiment, Lactilactobacillus curvatus and Lactilactobacillus sakei are the only bacterial strains in the composition.

In one embodiment, the disclosure provides a method for reducing or inhibiting the colonization of an animal by a pathogenic bacterium. In one embodiment, the disclosure provides a method for reducing or inhibiting the colonization of the gut or gastrointestinal tract (GIT) of an animal by a pathogenic bacterium. The method includes administering to an animal an effective amount of a probiotic composition described above. In an embodiment, the probiotic composition comprises a non-natural and unique combination of Lactilactobacillus bacteria strains as disclosed herein.

In one embodiment, the disclosure provides a method of treating necrotic enteritis in poultry by administering to poultry a probiotic composition described above and herein.

In one embodiment, the disclosure provides a method of delivering a metabolite to the gut of an animal. The method includes administering to an animal a probiotic composition having a first isolated Lactilactobacillus curvatus strain and a second isolated Lactilactobacillus strain described above and herein. In an embodiment, the metabolite is secreted by the combination of a first isolated Lactilactobacillus curvatus strain and a second isolated Lactilactobacillus strain.

In an embodiment, the composition is formulated as animal feed, feed additive, food ingredient, water additive, water-mixed additive, consumable solution, consumable spray additive, consumable solid, consumable gel, injection, or combinations thereof. In one embodiment, the composition comprises animal feed.

In an embodiment of the composition or method of the disclosure, the animal administered the composition further exhibits at least one improved gut characteristic, as compared to an animal not administered the composition; wherein improved gut characteristics includes at least one of: decreasing pathogen-associated lesion formation in the gastrointestinal tract, increasing feed digestibility, increasing meat quality, modulating microbiome, improving short chain fatty acids, and increasing gut health (reducing permeability and inflammation). The animal administered the composition can be a fish, in particular Salmo samar.

In an embodiment, the animal is human, non-human, poultry (chicken, turkey), bird, cattle, swine, fish, cat, or dog. In an embodiment the animal is fish. In an embodiment, the fish is salmon.

In an embodiment, the animal is fish and wherein the fish administered the composition further exhibits at least one of: decreased feed conversion ratio, increased weight, increased lean body mass, decreased pathogen-associated lesion formation in the gastrointestinal tract, decreased colonization of pathogens, modulated microbiome, increased feed digestibility, and decreased mortality rate, as compared to fish not administered the composition. In an embodiment, the fish is salmon and wherein the salmon administered the composition further exhibits at least one of: decreased feed conversion ratio, increased weight, increased lean body mass, decreased pathogen-associated lesion formation in the gastrointestinal tract, decreased colonization of pathogens, modulated microbiome, increased feed digestibility, and decreased mortality rate, as compared to salmon not administered the composition.

In an embodiment, the feed conversion ratio is decreased by at least 1%, at least 5%, at least 6%, at least 7%, at least 8%, at least 9%, at least 10%, or at least 15%. In an embodiment, poultry weight is increased by at least 1%, at least 5%, at least 10%, at least 15%, at least 25%, or at least 50%. In an embodiment, pathogen-associated lesion formation in the gastrointestinal tract is decreased by at least 1%, at least 5%, at least 10%, at least 15%, at least 25%, or at least 50%. In an embodiment, mortality rate is decreased by at least 1%, at least 5%, at least 10%, at least 15%, at least 25%, or at least 50%.

In an embodiment, the administration comprises spray administration. In an embodiment, the administration comprises dry feed administration. In an embodiment, the administration comprises immersion, intranasal, intramammary, topical, or inhalation.

In an embodiment, the administration comprises administration of a vaccine. In an embodiment, the animal is administered a vaccine prior to the administration of the composition. In an embodiment, the animal is a fish and the fish is administered a vaccine prior to the administration of the composition. In an embodiment, the fish is salmon and the salmon is administered a vaccine prior to the administration of the composition. In an embodiment, the animal is administered a vaccine concurrently with the administration of the composition. In an embodiment, the animal is a fish and the fish is administered a vaccine concurrently with the administration of the composition.

In an embodiment, a composition is provided for use in therapy. In an embodiment, a composition is provided for use in improving animal health. In an embodiment, a composition is provided for use in reducing colonization of an animal by a pathogenic bacterium. In an embodiment, a composition is provided for use in the manufacture of a medicament for reducing colonization of an animal by a pathogenic bacterium.

In an embodiment, a method is provided reducing mortality in fish, wherein the method comprises administering a composition as provided herein to a fish in need thereof. In an embodiment, a method is provided reducing mortality in salmon, wherein the method comprises administering a composition as provided herein to a salmon in need thereof. In an embodiment, a method is provided for improving performance selected from average daily feed intake (ADFI), average daily gain (ADG) and feed conversion ratio (FCR) in salmon, wherein the method comprises administering a composition as provided herein to a salmon in need thereof.

In an embodiment, a method is provided of preparing a fermentation product comprising the steps of:
(a) obtaining at least any combination of the following bacterial strains:
a Lactilactobacillus strain comprising SEQ ID NO: 1, a Lactilactobacillus strain comprising SEQ ID NO: 3, a Lactilactobacillus strain comprising SEQ ID NO: 2 or a Lactilactobacillus strain comprising SEQ ID NO: 4, and combinations thereof;
(b) contacting the at least one strain of step (a) with cell growth media;
(c) incubating a combination of at least one strain of step (a) and cell growth media of step (b) at a temperature of about 37° C. for an incubation time of about 24 hours; and
(d) cooling the combination of step (c);
wherein the product of step (d) comprises the fermentation product.

In a further embodiment, a method is provided of preparing a fermentation product comprising obtaining at least two of the bacterial strains.

In an embodiment, the cell growth media comprises: 0.5 g casamino acids/L, 1% glucose, Disodium Phosphate (anhydrous) 6.78 g/L, Monopotassium Phosphate 3 g/L, Sodium Chloride 0.5 g/L, and Ammonium Chloride 1 g/L.

In an embodiment, the cell growth media comprises: Peptone 30 g/L; Sucrose 30 g/L; Yeast extract 8 g/L; KH2PO4 4 g/L; MgSO4 1.0 g/L; and MnSO4 25 mg/L.

While there have been described what are presently believed to be particular embodiments of the present disclosure, those skilled in the art will realize that other and further changes and modifications may be made thereto without departing from the spirit of the disclosure, and it is intended to claim all such modifications and changes as come within the true scope of the disclosure.

Other objects and advantages will become apparent to those skilled in the art from a review of the ensuing detailed description, which proceeds with reference to the following illustrative drawings, and the attendant claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts the phylogenetic relationship of L. curvatus, L. sakei and L. fuchuensis strains using 92 core genes. The phylogenetic relationship was explored using UBCG v3.0 and a maximum likelihood tree was inferred using GTR+CAT model. L. reuteri was used as an outgroup.

FIG. 2 depicts the pan-genome analyses for L. curvatus, L. sakei and L. fuchuensis strains as determined by Orthofinder. A. The pan-genome pie chart showing gene content from core and accessory genes. B. A heatmap showing gene presence (dark blue) or absence (light blue) in each of the 18 strains. The core-genome tree generated was compared with a matrix where the core and accessory genes were either present or absent.

FIG. 3 depicts the antimicrobial susceptibility of Bacillus and Lactilactobacillus strains. A. Bacillus spp. and B. Lactilactobacillus spp. probiotic candidates. Minimum inhibitory concentration (MIC) (μg/mL) values for each antibiotic tested of respective genus are shown. Nine medically important antibiotics at a concentration range of 0.06-32 g/ml were tested and the respective antimicrobial susceptibility cut-off concentrations required for genus are shown at the bottom of each panel. *NR=not required by EFSA.

FIG. 4 depicts the effect of probiotic supplementation on the weights of salmon following daily administration in feed for 45 days. A. Description of study groups, dose and probiotic candidates tested. Negative control product (NCP), Test Product (TP) groups, CFU/g feed, organisms in consortia, text color corresponds to isolate origin; black=Indiana, grey=Chile, yellow=Norway. B. Body weights for each group following daily administration of the respective probiotic candidates in feed for 45 days. Horizontal bar denotes mean. *P=0.0039 for NCP vs TP3, Student's t-test (n=70). C. Timeline of experimental events.

FIG. 5 depicts the principal component analysis of feature abundance changes across media additives compared to media controls. Each marker in the figure represents one of three replicates in the corresponding treatment (different colors). Numbers in parenthesis indicate the variance explained by each of the principal components. The histogram on the bottom represents the distribution of samples from each of the two strains along the first principal component.

FIG. 6 depicts the features showing at least 10-fold difference in abundance with different media additives compared to a control condition. A. The number of features with a higher than 10-fold increase or decrease in abundance in media supplemented with different additives compared to a glucose media control for strain PTA-17. Error bars represent the standard error of the mean across replicates (n=3). B. Like A, but for strain PTA-16. In both panels, additives are sorted according to the number of metabolites with increased abundance.

FIG. 7 depicts the two-component PCA for standardized feature abundances in PTA-17 and PTA-16. Each marker in the figure represents the mean of three replicates for each strain and growth condition. Abundance data for each feature was Z-score standardized before PCA analysis. Numbers in parenthesis indicate the variance explained by each of the principal components.

FIG. 8 depicts the MeSH terms in different categories associated with potential compounds produced by PTA-17 and PTA-16. MeSH terms associated via co-annotation in PubMed publications were classified based on the MeSH term ontology. Numbers for adenine and biotin were excluded.

FIG. 9 depicts the number of MeSH terms associated with possible metabolites produced by PTA-17 and PTA-16. MeSH terms were identified by their significant co-annotation with metabolites across PubMed articles. Black stars indicate metabolites associated with features enriched by feed additives in PTA-16 but not PTA-17, red stars indicate features enriched in LcELA100 but not LcELA093.

FIG. 10A provides a schematic overview of group allocation to tanks and replicates in a study of probiotics on Atlantic salmon (Salmo salar) and B depicts the study design for evaluating supplementation of probiotics on Atlantic salmon (Salmo salar) in the presence of Salmonid Rickettsial Syndrome (SRS). Negative Control is standard diet only; TP1 is B. amyloliquefaciens Strain A+B. subtilis Strain A+B. amyloliquefaciens Strain B+Standard diet; TP2 is B. velezensis Strain A+B. subtilis Strain B+B. subtilis Strain A+Standard diet; TP3 is L. curvatus ELA204100+L. sakei ELA204093+Standard diet; TP4 is L. curvatus ELA204100+L. sakei ELA204093+B. subtilis Strain A+Standard diet; TP5 is B. velezensis Strain A+B. subtilis Strain B+L. curvatus ELA204100+Standard diet.

FIGS. 11A and B depicts average group weights over time. A depicts average body weight (BW) in grams and B depicts average body weight vs normal or negative control (NCP). Negative Control is standard diet only; TP1 is B. amyloliquefaciens Strain A+B. subtilis Strain A+B. amyloliquefaciens Strain B+Standard diet; TP2 is B. velezensis Strain A+B. subtilis Strain B+B. subtilis Strain A+Standard diet; TP3 is L. curvatus ELA204100+L. sakei ELA204093+Standard diet; TP4 is L. curvatus ELA204100+L. sakei ELA204093+B. subtilis Strain A+Standard diet; TP5 is B. velezensis Strain A+B. subtilis Strain B+L. curvatus ELA204100+Standard diet.

FIG. 12A-D depicts average tank weights. (A) shows average body weight (BW) in grams at day 1, (B) shows average body weight (BW) in grams at day 18, (C) shows average body weight (BW) in grams at day 33 and (D) shows average body weight (BW) in grams at day 45. Negative Control (NCP) is standard diet only; TP1 is B. amyloliquefaciens Strain A+B. subtilis Strain A+B. amyloliquefaciens Strain B+Standard diet; TP2 is B. velezensis Strain A+B. subtilis Strain B+B. subtilis Strain A+Standard diet; TP3 is L. curvatus ELA204100+L. sakei ELA204093+Standard diet; TP4 is L. curvatus ELA204100+L. sakei ELA204093+B. subtilis Strain A+Standard diet; TP5 is B. velezensis Strain A+B. subtilis Strain B+L. curvatus ELA204100+Standard diet.

FIGS. 13A and B depicts all weights at the end of Phase 1 or at day 45. Negative Control (NCP) is standard diet only; TP1 is B. amyloliquefaciens Strain A+B. subtilis Strain A+B. amyloliquefaciens Strain B+Standard diet; TP2 is B. velezensis Strain A+B. subtilis Strain B+B. subtilis Strain A+Standard diet; TP3 is L. curvatus ELA204100+L. sakei ELA204093+Standard diet; TP4 is L. curvatus ELA204100+L. sakei ELA204093+B. subtilis Strain A+Standard diet; TP5 is B. velezensis Strain A+B. subtilis Strain B+L. curvatus ELA204100+Standard diet.

FIG. 14 provides a tabulation of top probiotic sample information. Each of Lactilactobacillus strain 93 (ELA204093), Lactilactobacillus strain 100 (ELA204100), Lactilactobacillus strain 388 (ELA214388) and Lactilactobacillus strain 391 (ELA214391) are shown.

FIG. 15A provides a schematic overview of group allocation to tanks and replicates in a study of Lactilactobacillus probiotics on Atlantic salmon (Salmo salar) and B depicts the study design for evaluating supplementation of probiotics on Atlantic salmon (Salmo salar) Negative Control is standard diet only; TP1 is L. curvatus ELA204100+L. curvatus ELA204093+Standard diet; TP2 is L. curvatus ELA214388+L. sakei ELA214391+Standard diet.

FIG. 16 shows the effect of probiotic supplementation on the weights of salmon following daily administration in feed for 75 days in saltwater. FIG. 16A provides a timeline of experimental events. FIG. 16B shows mean body weights±standard error for each group following daily administration of the respective probiotic candidates in feed for 75 days. P=0.041 for NCP-S vs TP1-S, ANOVA with Dunnett's test (n=64)

FIG. 17 shows cumulative mortality after a P. salmonis cohabitation challenge on test (TP1, TP2) and control fish (NCP).

FIG. 18: Cumulative mortality after a P. salmonis cohabitation challenge on test, control and shedder fish. Each colour corresponds to a given tank, with solid line displaying shedders and sotted line displaying test fish. Analysis of survival (performed on survival curves, not shown, denotes a significant differences between NCP and TP1 (p=0.01).

### DETAILED DESCRIPTION

In an embodiment, the disclosure provides for a composition that is a combination of two Lactilactobacillus strains, particularly two isolated Lactilactobacillus curvatus strains or an isolated Lactilactobacillus curvatus strain and an isolated Lactilactobacillus sakei strain, wherein the composition includes a carrier that is suitable for animal consumption or use.

Without wishing to be bound by theory, it is believed that the consortia of strains described above have a unique secretion profile that provides health benefits to an animal when they colonize the gastrointestinal tract of an animal. Furthermore, it is believed that the combination of a first isolated Lactilactobacillus curvatus strain and a second isolated Lactilactobacillus strain, as described above, provide a unique combined metabolite secretion profile that provides health benefits to an animal when they colonize the gastrointestinal tract of an animal.

Even further, it is believed that the combination of a first isolated and a second isolated Lactilactobacillus strain as described above and herein, provide a unique combined metabolite secretion profile that provides health benefits to an animal when they colonize the gastrointestinal tract of an animal.

As used herein and in the context of bacterial consortia, “unique metabolites” include metabolites that are secreted at least 1.5, at least 2 fold, at least 3 fold, at least 5 fold, or at least 10 fold greater as compared to secretion of the respective metabolite by the bacterial strain grown individually.

The composition may include or comprise live bacteria or bacterial spores, or a combination thereof.

In some embodiments, the composition does not include antibiotics. Exemplary antibiotics include tetracycline, bacitracin, tylosin, salinomycin, virginiamycin and bambermycin.

In some embodiments, the Lactilactobacillus strains of the present disclosure are not genetically engineered or genetically modified and do not contain heterologous genetic sequences.

The compositions described above may include a carrier suitable for animal consumption or use. Examples of suitable carriers include edible food grade material, mineral mixture, gelatin, cellulose, carbohydrate, starch, glycerin, water, glycol, molasses, corn oil, animal feed, such as cereals (barley, maize, oats, and the like), starches (tapioca and the like), oilseed cakes, and vegetable wastes. In some embodiments, the compositions include vitamins, minerals, trace elements, emulsifiers, aromatizing products, binders, colorants, odorants, thickening agents, and the like.

In some embodiments, the compositions include one or more biologically active molecule or therapeutic molecule. Examples of the aforementioned include ionophore; vaccine; antibiotic; antihelmintic; virucide; nematicide; amino acids such as methionine, glycine, and arginine; fish oil; krill oil; and enzymes.

In some embodiments, the compositions or combinations may additionally include one or more prebiotic. In some embodiments, the compositions may be administered along with or may be coadministered with one or more prebiotic. Prebiotics may include organic acids or non-digestible feed ingredients that are fermented in the lower gut and may serve to select for beneficial bacteria. Prebiotics may include mannan-oligosaccharides, fructo-oligosaccharides, galacto-oligosaccharides, chito-oligosaccharides, isomalto-oligosaccharides, pectic-oligosaccharides, xylo-oligosaccharides, and lactose-oligosaccharides.

The composition may be formulated as animal feed, feed additive, food ingredient, water additive, water-mixed additive, consumable solution, consumable spray additive, consumable solid, consumable gel, injection, or combinations thereof. The composition may be formulated and suitable for use as or in one or more of animal feed, feed additive, food ingredient, water additive, water-mixed additive, consumable solution, consumable spray additive, consumable solid, consumable gel, injection, or combinations thereof. The composition may be suitable and prepared for use as animal feed, feed additive, food ingredient, water additive, water-mixed additive, consumable solution, consumable spray additive, consumable solid, consumable gel, injection, or combinations thereof.

### Methods and Methods of Use

In some embodiments, the disclosure provides for the use of any of the compositions described above to improve a phenotypic trait of interest in an animal. As used herein, a probiotic is a composition that improves a phenotypic trait of interest in an animal.

In all embodiments of the disclosure, an animal may include a farmed animal or livestock or a domesticated animal. Livestock or farmed animal may include cattle (e.g. cows or bulls (including calves)), poultry (including broilers, chickens and turkeys), pigs (including piglets), birds, aquatic animals such as fish, agastric fish, gastric fish, freshwater fish such as salmon, cod, trout and carp, e.g. koi carp, marine fish such as sea bass, and crustaceans such as shrimps, mussels and scallops), horses (including race horses), sheep (including lambs). A domesticated animal may be a pet or an animal maintained in a zoological environment and may include any relevant animal including canines (e.g. dogs), felines (e.g. cats), rodents (e.g. guinea pigs, rats, mice), birds, fish (including freshwater fish and marine fish), and horses. The animal may be a pregnant or breeding animal.

Examples of improving a phenotypic trait includes decreasing pathogen-associated lesion formation in the gastrointestinal tract, decreasing colonization of pathogens, increasing feed digestibility, modulating microbiome, increasing short chain fatty acids, and increasing gut health or characteristic (reducing permeability and inflammation).

A pathogen may be a bacteria or a virus. The virus may include a pathogenic virus infecting animals, including livestock animals or domesticated animals and may be specific for a particular animal such as a fish virus or a salmon virus. The bacteria may include a pathogenic bacteria infecting animals, including fish and may be specific for a particular animal such as a fish bacteria or a salmon bacteria.

The compositions may be used to treat an infection particularly a bacterial infection. In some aspects, the compositions described above are used to treat an infection from at least one of Piscirikettsia salmonis and Tanacibaculum maritimum. The compositions may be used to inhibit infection, particularly a bacterial infection. Infection may be by one or more Piscirikettsia salmonis and Tanacibaculum maritimum.

In some aspects, the compositions described above are used to reduce colonization by or inhibit colonization by a bacteria in an animal, particularly in a herd or group of animals, particularly of pathogenic bacteria. In some aspects, the compositions described above are used to reduce colonization by or inhibit colonization of at least one of Piscirikettsia salmonis and Tanacibaculum maritimum.

In some aspects, the compositions described above are used to reduce transmission of bacteria, particularly pathogenic bacteria, in an animal pen or in a group or herd of animals. In some aspects, the compositions described above are used to reduce transmission in an animal pen or in a group or herd of animals of at least one of Piscirikettsia salmonis and Tanacibaculum maritimum.

In some aspects, the compositions described above are used to reduce bacterial load, particularly pathogenic bacteria or clinically significant bacteria, including the number or amount of bacteria in the gut or gastrointestinal tract of an animal.

In some aspects, the compositions described above are used to treat at least one of inflammatory bowel disease, obesity, liver abscess, ruminal acidosis, leaky gut syndrome, piglet diarrhea, necrotic enteritis, coccidiosis, salmon ricketsial septicemia, and foodborne diseases.

In one embodiment, examples of phenotypic traits of interest in animals include decreased feed conversion ratio, increased weight, increased lean body mass, decreased pathogen-associated lesion formation in the gastrointestinal tract, decreased colonization of pathogens, modulated microbiome, increased egg quality, increased feed digestibility, and decreased mortality rate, as compared to animals not administered the composition.

In one embodiment, examples of phenotypic traits of interest in poultry include decreased feed conversion ratio, increased weight, increased lean body mass, decreased pathogen-associated lesion formation in the gastrointestinal tract, decreased colonization of pathogens, modulated microbiome, increased egg quality, increased feed digestibility, and decreased mortality rate, as compared to poultry not administered the composition.

In one embodiment, examples of phenotypic traits of interest in swine include decreased feed conversion ratio, increased weight, increased lean body mass, decreased pathogen-associated lesion formation in the gastrointestinal tract, decreased colonization of pathogens, modulated microbiome, increased feed digestibility, prevention of or reduction of post-weaning diarrhea in piglets, reduction of fecal scores, increased piglet body weight or weight gain, reduced unconsumed feed, increased daily feed intake, improved weight gain to feed ratio and decreased mortality rate, as compared to swine not administered the composition.

Methods are provided herein for reduction of post-weaning diarrhea in an animal. Methods are provided herein for reduction of fecal scores in a herd or group or pen of animals. Methods are provided herein for increase in body weight, for weight gain, for reducing unconsumed feed, for increasing daily feed intake, or for improving weight gain to feed ratio in a animal or in a herd or group or pen of animals.

In some aspects, the animal administered an effective amount of the composition disclosed herein exhibits a decrease in the feed conversion ratio by at least 1%, at least 5%, at least 6%, at least 7%, at least 8%, at least 9%, at least 10%, or at least 15%. In some aspects, the poultry administered an effective amount of the composition disclosed herein exhibits a decrease in the feed conversion ratio by at least 1%, at least 5%, at least 6%, at least 7%, at least 8%, at least 9%, at least 10%, or at least 15%. In some aspects, the swine or pigs/piglets administered an effective amount of the composition disclosed herein exhibits a decrease in the feed conversion ratio by at least 1%, at least 5%, at least 6%, at least 7%, at least 8%, at least 9%, at least 10%, or at least 15%.

In some aspects, the animal administered an effective amount of the composition disclosed herein exhibits an increase in animal weight by at least 1%, at least 5%, at least 25%, 20% or at least 50%. In some aspects, the poultry administered an effective amount of the composition disclosed herein exhibits an increase in poultry weight by at least 1%, at least 5%, at least 25%, 20% or at least 50%. In some aspects, the swine or piglet administered an effective amount of the composition disclosed herein exhibits an increase in swine or piglet weight by at least 1%, at least 5%, at least 25%, 20% or at least 50%.

In some aspects, the animal administered an effective amount of the composition disclosed herein exhibits a decrease in pathogen-associated lesion formation in the gastrointestinal tract by at least 1%, at least 5%, at least 25%, or at least 50%. In some aspects, the poultry administered an effective amount of the composition disclosed herein exhibits a decrease in pathogen-associated lesion formation in the gastrointestinal tract by at least 1%, at least 5%, at least 25%, or at least 50%. In some aspects, the swine or piglet administered an effective amount of the composition disclosed herein exhibits a decrease in pathogen-associated lesion formation in the gastrointestinal tract by at least 1%, at least 5%, at least 25%, or at least 50%.

In some aspects, the animal administered an effective amount of the composition disclosed herein exhibits decrease in the mortality rate by at least 1%, at least 5%, at least 25%, or at least 50%. In some aspects, the poultry administered an effective amount of the composition disclosed herein exhibits decrease in the mortality rate by at least 1%, at least 5%, at least 25%, or at least 50%. In some aspects, the swine, piglet administered an effective amount of the composition disclosed herein exhibits decrease in the mortality rate by at least 1%, at least 5%, at least 25%, or at least 50%.

In some aspects, the poultry administered an effective amount of the composition exhibits an increase in production efficiency by at least 6.0%, by at least 7%, by at least 10%, or by at least 15%.

The compositions may further include one or more component or additive. The one or more component or additive may be a component or additive to facilitate administration, for example by way of a stabilizer or vehicle, or by way of an additive to enable administration to an animal such as by any suitable administrative means, including in aerosol or spray form, in water, in feed or in an injectable form. Administration to an animal may be by any known or standard technique. These include oral ingestion, gastric intubation, or broncho-nasal spraying. The compositions disclosed herein may be administered by immersion, intranasal, intramammary, topical, mucosally, or inhalation.

Compositions may include a carrier in which the bacterium or any such other components is suspended or dissolved. Such carrier(s) may be any solvent or solid or encapsulated in a material that is non-toxic to the inoculated animal and compatible with the organism. Suitable pharmaceutical carriers include liquid carriers, such as normal saline and other non-toxic salts at or near physiological concentrations, and solid carriers, such as talc or sucrose and which can also be incorporated into feed for farm animals. When used for administering via the bronchial tubes, the composition is presented in particular in the form of an aerosol. A dye may be added to the compositions hereof, including to facilitate chacking or confirming whether an animal has ingested or breathed in the composition.

When administering to animals, including farm animals, administration may include orally or by injection. Oral administration can include by bolus, tablet or paste, or as a powder or solution in feed or drinking water. The method of administration will often depend on the species being fed or administered, the numbers of animals being fed or administered, and other factors such as the handling facilities available and the risk of stress for the animal.

The dosages required will vary and need be an amount sufficient to induce an immune response or to effect a biological or phenotypic change or response expected or desired. Routine experimentation will establish the required amount. Increasing amounts or multiple dosages may be implemented and used as needed.

In an embodiment of the disclosure, the bacterial strains are administered in doses indicated as CFU/g or colony forming units of bacteria per gram. In an embodiment, the dose is in the range of 1×10³ to 1×10⁹ CFU/g. In an embodiment, the dose is in the range of 1×10³ to 1×10⁷. In an embodiment, the dose is in the range of 1×10⁴ to 1×10⁶. In an embodiment, the dose is in the range of 5×10⁴ to 1×10⁶. In an embodiment, the dose is in the range of 5×10⁴ to 6×10⁵. In an embodiment, the dose is in the range of 7×10⁴ to 3×10⁵. In an embodiment, the dose is approximately 50K, 75K, 100K, 125K, 150K, 200K, 300K, 400K, 500K, 600K CFU/g.

Administration of the compositions disclosed herein may include co-administration with a vaccine or therapeutic compound. Administration of the vaccine or therapeutic compound includes administration prior to, concurrently, or after the composition disclosed herein.

Suitable vaccines in accordance with this embodiment include a vaccine that aids in the prevention of coccidiosis.

In some embodiments, the methods described above are administered to an animal in the absence of antibiotics.

### Definitions

Unless defined otherwise, all technical and scientific terms used herein have the same meaning as those commonly understood by one of ordinary skill in the art to which this disclosure belongs.

As used in the description herein and throughout the claims that follow, the meaning of “a,” “an,” and “the” includes plural reference unless the context clearly dictates otherwise.

As used herein, “isolated” means that the subject isolate has been separated from at least one of the materials with which it is associated in a particular environment, for example, its natural environment.

Thus, an “isolate” does not exist in its naturally occurring environment; rather, it is through the various techniques known in the art that the microbe has been removed from its natural setting and placed into a non-naturally occurring state of existence. Thus, the isolated strain or isolated microbe may exist as, for example, a biologically pure culture in association with an acceptable carrier.

As used herein, “individual isolates” should be taken to mean a composition, or culture, comprising a predominance of a single species, or strain, of microorganism, following separation from one or more other microorganisms. The phrase should not be taken to indicate the extent to which the microorganism has been isolated or purified. However, “individual isolates” can include substantially only one species, or strain, of microorganism.

As used herein, the term “bacterial consortia”, “bacterial consortium”, “microbial consortia” or “microbial consortium” refers to a subset of a microbial community of individual microbial species, or strains of a species, which can be described as carrying out a common function, or can be described as participating in, or leading to, or correlating with, a recognizable parameter, such as a phenotypic trait of interest (e.g., increased feed efficiency in poultry). The community may comprise two or more species, or strains of a species, of microbes. In some instances, the microbes coexist within the community symbiotically.

As used herein, “spore” or “spores” refer to structures produced by bacteria that are adapted for survival and dispersal. Spores are generally characterized as dormant structures; however, spores are capable of differentiation through the process of germination. Germination is the differentiation of spores into vegetative cells that are capable of metabolic activity, growth, and reproduction. The germination of a single spore results in a single bacterial vegetative cell. Bacterial spores are structures for surviving conditions that may ordinarily be nonconducive to the survival or growth of vegetative cells.

As used herein, the terms “colonize” and “colonization” include “temporarily colonize” and “temporary colonization”.

As used herein, “microbiome” refers to the collection of microorganisms that inhabit the gastrointestinal tract of an animal and the microorganisms' physical environment (i.e., the microbiome has a biotic and physical component). The microbiome is fluid and may be modulated by numerous naturally occurring and artificial conditions (e.g., change in diet, disease, antimicrobial agents, influx of additional microorganisms, etc.). The modulation of the gastrointestinal microbiome can be achieved via administration of the compositions of the disclosure can take the form of: (a) increasing or decreasing a particular Family, Genus, Species, or functional grouping of a microbe (i.e., alteration of the biotic component of the gastrointestinal microbiome) and/or (b) increasing or decreasing gastrointestinal pH, increasing or decreasing volatile fatty acids in the gastrointestinal tract, increasing or decreasing any other physical parameter important for gastrointestinal health (i.e., alteration of the abiotic component of the gut microbiome).

As used herein, “probiotic” refers to a substantially pure microbe (i.e., a single isolate) or a mixture of desired microbes, and may also include any additional components (e.g., carrier) that can be administered to an animal to provide a beneficial health effect. Probiotics or microbial compositions of the disclosure may be administered with an agent or carrier to allow the microbes to survive the environment of the gastrointestinal tract, i.e., to resist low pH and to grow in the gastrointestinal environment.

The term “growth medium” as used herein, is any medium which is suitable to support growth of a microbe. By way of example, the media may be natural or artificial including gastrin supplemental agar, minimal media, rich media, LB media, blood serum, and tissue culture gels. It should be appreciated that the media may be used alone or in combination with one or more other media. It may also be used with or without the addition of exogenous nutrients.

As used herein, “improved” should be taken broadly to encompass improvement of a characteristic of interest, as compared to a control group, or as compared to a known average quantity associated with the characteristic in question. For example, “improved” feed efficiency associated with application of a beneficial microbe, or microbial ensemble, of the disclosure can be demonstrated by comparing the feed efficiency of poultry treated by the microbes taught herein to the feed efficiency of poultry not treated. In the present disclosure, “improved” does not necessarily demand that the data be statistically significant (i.e. p<0.05); rather, any quantifiable difference demonstrating that one value (e.g. the average treatment value) is different from another (e.g. the average control value) can rise to the level of “improved.”

As used herein, the term “metabolite” refers to an intermediate or product of metabolism. In some embodiments, a metabolite includes a small molecule. Metabolites have various functions, including in fuel, structural, signaling, stimulatory and inhibitory effects on enzymes, as a cofactor to an enzyme, in defense, and in interactions with other organisms (such as pigments, odorants and pheromones). A primary metabolite is directly involved in normal growth, development and reproduction. A secondary metabolite is not directly involved in these processes but usually has an important ecological function. Examples of metabolites include but are not limited to antibiotics and pigments such as resins and terpenes, etc. Metabolites, as used herein, include small, hydrophilic carbohydrates; large, hydrophobic lipids and complex natural compounds.

As used herein, “carrier”, “acceptable carrier”, or “pharmaceutical carrier” are used interchangeably and refer to a diluent, adjuvant, excipient, or vehicle with which the compound is administered. Such carriers can be sterile liquids, such as water and oils, including those of petroleum, animal, vegetable, or synthetic origin; such as peanut oil, soybean oil, mineral oil, sesame oil, and the like. Water or aqueous solution saline solutions and aqueous dextrose and glycerol solutions are employed in particular as carriers, in some embodiments as injectable solutions. Alternatively, the carrier can be a solid dosage form carrier, including but not limited to one or more of a binder (for compressed pills), a glidant, an encapsulating agent, a flavorant, and a colorant. The choice of carrier can be selected with regard to the intended route of administration and standard pharmaceutical practice. See Handbook of Pharmaceutical Excipients, (Sheskey, Cook, and Cable) 2017, 8th edition, Pharmaceutical Press; Remington's Pharmaceutical Sciences, (Remington and Gennaro) 1990, 18th edition, Mack Publishing Company; Development and Formulation of Veterinary Dosage Forms (Hardee and Baggot), 1998, 2nd edition, CRC Press.

As used herein, “delivery” or “administration” means the act of providing a beneficial activity to a host. The delivery may be direct or indirect. An administration could be by an oral, nasal, or mucosal route. For example without limitation, an oral route may be an administration through drinking water, a nasal route of administration may be through a spray or vapor, and a mucosal route of administration may be through direct contact with mucosal tissue. Mucosal tissue is a membrane rich in mucous glands such as those that line the inside surface of the nose, mouth, esophagus, trachea, lungs, stomach, gut, intestines, and anus. In the case of birds, administration may be in ovo, i.e. administration to a fertilized egg. In ovo administration can be via a liquid which is sprayed onto the egg shell surface, or an injected through the shell.

As used herein, the terms “treating”, “to treat”, or “treatment”, include restraining, slowing, stopping, inhibiting, reducing, ameliorating, or reversing the progression or severity of an existing symptom, disorder, condition, or disease. A treatment may also be applied prophylactically to prevent or reduce the incidence, occurrence, risk, or severity of a clinical symptom, disorder, condition, or disease.

As used herein, “animal” includes bird, poultry, a human, or a non-human mammal. Specific examples include chickens, turkey, dogs, cats, cattle, salmon, fish, swine and horse. The chicken may be a broiler chicken, egg-laying, or egg-producing chicken. As used herein, the term “poultry” includes domestic fowl, such as chickens, turkeys, ducks, and geese.

As used herein, “gut” refers to the gastrointestinal tract including stomach, small intestine, and large intestine. The term “gut” may be used interchangeably with “gastrointestinal tract”.

As used herein Lactobacillus and Lactilactobacillus are used interchangeably, the latter being the more modern term.

Any examples or illustrations given herein are not to be regarded in any way as restrictions on, limits to, or express definitions of any term or terms with which they are utilized. Instead, these examples or illustrations are to be regarded as being described with respect to one particular embodiment and as being illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example,” “for instance,” “e.g.,” and “in one embodiment.” In this specification, groups of various parameters containing multiple members are described. Within a group of parameters, each member may be combined with any one or more of the other members to make additional sub-groups. For example, if the members of a group are a, b, c, d, and e, additional sub-groups specifically contemplated include any one, two, three, or four of the members, e.g., a and c; a, d, and e; b, c, d, and e; etc.

Throughout this specification, quantities are defined by ranges, and by lower and upper boundaries of ranges. Each lower boundary can be combined with each upper boundary to define a range. The lower and upper boundaries should each be taken as a separate element. Two lower boundaries or two upper boundaries may be combined to define a range.

### DEPOSIT INFORMATION

Lactilactobacillus curvatus strain “ELA204093” was deposited on Aug. 31, 2021 according to the Budapest Treaty in the American Type Culture Collection (ATCC), ATCC Patent Depository, 10801 University Boulevard, Manassas, Va., 20110, USA. The deposit has been assigned ATCC Patent Deposit Number PTA-127116.

Lactilactobacillus curvatus strain “ELA204100” was deposited on Aug. 31, 2021 according to the Budapest Treaty in the American Type Culture Collection (ATCC), ATCC Patent Depository, 10801 University Boulevard, Manassas, Va., 20110, USA. The deposit has been assigned ATCC Patent Deposit Number PTA-127117.

Lactilactobacillus curvatus strain “ELA214388” was deposited on Aug. 31, 2021 according to the Budapest Treaty in the American Type Culture Collection (ATCC), ATCC Patent Depository, 10801 University Boulevard, Manassas, Va., 20110, USA. The deposit has been assigned ATCC Patent Deposit Number PTA-127118.

Lactilactobacillus sakei strain “ELA214391” was deposited on Aug. 31, 2021 according to the Budapest Treaty in the American Type Culture Collection (ATCC), ATCC Patent Depository, 10801 University Boulevard, Manassas, Va., 20110, USA. The deposit has been assigned ATCC Patent Deposit Number PTA-127119.

Access to the deposits will be available during the pendency of this application to persons determined by the Commissioner of Patents and Trademarks to be entitled thereto under 37 C.F.R. § 1.14 and 35 U.S.C. § 122. Upon allowance of any embodiments in this application, all restrictions on the availability to the public of the variety will be irrevocably removed.

The deposits will be maintained in the ATCC depository, which is a public depository, for a period of 30 years, or 5 years after the most recent request, or for the effective life of the patent, whichever is longer, and will be replaced if a deposit becomes nonviable during that period.

The present disclosure may be better understood with reference to the examples, set forth below. The following examples are put forth so as to provide those of ordinary skill in the art with a complete disclosure and description of how the compounds, compositions, and/or methods claimed herein are made and evaluated, and are intended to be purely exemplary and are not intended to limit the disclosure. It will be appreciated that other embodiments and uses will be apparent to those skilled in the art and that the invention is not limited to these specific illustrative examples or specific embodiments.

### Aquaculture Probiotic Compositions

Salmon is a valuable protein source worldwide; since 2016, it has been the second-most popular seafood consumed in the United States. Salmon farming is critical to fill this demand as aquaculture provides 70% of global salmon production. Atlantic salmon are attractive to farmers as prices and profit margins are high due to strong demand, and require significantly less fresh water, space, and feed to produce the same mass of protein than terrestrial agriculture. The bulk of the salmon production cycle takes place in non-potable saltwater. Atlantic salmon meat is also attractive to consumers for its nutritional value including omega-3 fatty acids. However, the Atlantic salmon production cycle is relatively long at three years, which can make salmon farming capital-intensive and volatile. In an effort to improve feed efficiency, plant and insect proteins have been integrated into salmon feed. This can result in gut inflammation and poor weight gain due to antinutrients and changes in the fatty acid profile of salmon meat, specifically decreasing desirable omega-3 long-chain fatty acids. Intensive salmon farming can also be plagued by losses from disease like sea lice.

Lactobacilli are among the most widely used probiotic genus in human food and dietary supplements and are increasingly used as feed additives in aquaculture. Probiotics native to the target species, instead of species from a different environment may be better adapted to the aquatic environment and offer superior benefits to salmon. The inventors isolated and identified 900 native microbial isolates including 18 Lactobacilli from farmed salmon intestines.

Based on whole-genome sequencing and phylogenetic analysis, the Lactobacillus candidates belonged to Lactilactobacillus curvatus (L. curvatus) species and formed two distinct phylogenetic groups.

Using bioinformatics and in vitro analyses, two Lactilactobacillus strains were selected, L. curvatus ATCC PTA-127116 and L. curvatus ATCC PTA-127117, which showed desirable safety and probiotic properties. The two L. curvatus strains were evaluated for safety and efficacy in Atlantic salmon alongside spore-forming Bacilli isolated from salmon, poultry, and swine. All of the tested strains were safe to salmon with no adverse effects. While the inventors did not observe any efficacy in any Bacillus supplemented groups, the group administered with the two L. curvatus strains consortium in feed for seven weeks showed surprisingly significant improvement by 4.2% in final body weight compared to untreated group.

Comprehensive metabolomics analyses of the two strains in the presence of different prebiotics and/or additives revealed distinct metabolite profiles for each strain and prebiotic and/or additive with galactooligosaccharide-zinc-vitamin D3 combination resulting in most metabolic changes.

The two endogenous L. curvatus strains were identified for use in probiotic compositions and methods of use for Atlantic salmon. The two Lactilactobacillus curvatus (L. curvatus) strains may be used in combination, or individually, in probiotic compositions, optionally including prebiotics and/or additives to enhance their efficacy. Probiotics may be used to improve salmon weight gain and disease resistance, major challenges in aquaculture.

Lactobacillus, first bacteriologically described in 1901, is a popular probiotic candidate genus of lactic acid bacteria with a long history of safe use, and many studies have shown their efficacy in modulating terrestrial host immune systems. They dominate the intestine of healthy fish and favorably modulate fish gut microbiome. Lactobacilli improve fish disease resistance via immunostimulation. This effect likely stems from a combination of mechanisms such as humoral immune modulation, bacteriocin production, and lymphocyte modulation. Lactic acid bacteria can directly inhibit aquatic pathogens like Aeromonas, and when combined with prebiotics, form a synbiotic which can also improve humoral immune response and weight gain.

Various terrestrial and aquatic sources can yield probiotics for use in aquaculture, including cheese, humans, dairy, crops and soil, as well as recirculating aquaculture systems (RAS) and native fish specimens. Native microbiome species are already adapted to the temperature, pH, osmotic pressure, and native antimicrobial activity seen in aquaculture. While terrestrial probiotic candidates may be able to survive under these conditions, native species may already be optimized to conferring probiotic benefits, and colonization and positive effects may last longer.

The disclosure screened, identified, and analyzed native Lactilactobacillus candidates, recently differentiated within Lactobacillus for probiotic use in salmon. The study included whole genome sequencing feature analysis, as well as extensive metabolomics analysis in the presence of several prebiotic candidates toward the design of a synbiotic. It was surprisingly discovered that the combination of the Lactilactobacillus strains produced a synergistic effect, significant improvement in salmon growth performance.

The disclosure is now described by Examples which should not be used to unduly limit the disclosure to particular features or embodiments.

### EXAMPLES

### Example 1

### Identification of Probiotics

### Probiotic Candidate Isolation

Probiotic candidates were isolated from healthy salmon samples received from Chile, Norway, and North America over a seven-month period. On each site, selected stock fish were humanely euthanized according to the farm's standard husbandry procedures, e.g., overdose of an approved fish anesthetic, before packaging whole or processing for tissues prior to cold chain shipment.

On site or upon receipt, fish whole gut was excised, and the samples were separated aseptically into foregut and hindgut. Each sample was homogenized completely by hand in Whirl-Paks (Whirl-Pak; Madison, WI) in De Man Rogosa and Sharpe broth (MRS) (Becton Dickinson (BD); Franklin Lakes, NJ). Aliquots were heat-treated at 100° C. at 15° C. for 10 minutes to select for spore formers, targeting Bacillus spp. Dilutions were prepared to 10⁻² in PBS (Gibco Thermo Fisher; Hampton, NH) and 0.1 mL of each dilution was spread over the surface of plates of MRS agar (BD) supplemented with amphotericin B (Thermo Fisher) for lactic acid bacteria, and LB agar (BD) for Bacillus species. LB plates were incubated aerobically at 15° C. for three days, and MRS Plates were incubated at 15° C. or 23° C. under microaerophilic conditions in a GasPak EZ Campy Container system (BD) for 4 days before colonies were picked and re-isolated on fresh medium three times (Table 2).

TABLE 2
List of media conditions used in the study.

| Study | Condition | | |
|---|---|---|---|
| identifier | name | Role | Description |
| GLC | Glucose | Carbon source | Glucose |
| MALT | Maltose | Carbon source | Maltose |
| LAC | Lactose | Carbon source | Lactose |
| FUC | Fucose | Carbon source | Fucose |
| NAG | NAG | Simulant of | N-acetyl-glucosamine |
| | | fungal | |
| | | coculture | |
| BS | Bile Salt | Microbiome | Bile-salts |
| | | metabolites | |
| GOS | GOS | Prebiotic, | Galactooligosaccharide |
| | | carbon source | |
| IN | Inulin | Prebiotic, | Inulin (from chicory) |
| | | carbon source | |
| GOSC | GOS + vitC | Prebiotic, | Galactooligosaccharide |
| | | carbon source | and vitamin C |
| GOSD | GOS + vitD3 | Prebiotic, | Galactooligosaccharide |
| | | carbon source | and vitamin D3 |
| GOSZ | GOS + Zn | Prebiotic, | Galactooligosaccharide |
| | | carbon source | and zinc |
| GOSCMB | GOS combo | Prebiotic, | Galactooligosaccharide |
| | | carbon source | vitamin D3, |
| | | | and zinc |

Lactic acid bacteria were passaged under both aerobic and microaerophilic conditions at 15° C. and 23° C. Three of the candidates used in salmon studies were Bacillus isolated in the same way from chicken cecum and swine intestine described previously. Susanti et al. Front Microbiol. (2021) 12:747845; Latorre et al. Front Vet. Sci. (2016) 3:95.

### Bacterial Identification

Probiotic candidate strains were identified using 16S rRNA sequencing. Briefly, lactic acid bacterial strains were grown on Lactobacilli MRS agar for 36-48 hours under microaerobic conditions at 25° C. using BD GasPak container and sachets (BD). Bacillus strains were grown on LB agar for 36-48 hours under aerobic conditions at 25° C. Patched colonies were resuspended in 50 p. L of nuclease-free water and heated at 100° C. for 10 minutes. The debris were pelleted by brief centrifugation and the supernatant was used as a template for PCR. Sanger sequencing was sent to TacGen for analysis (TacGen; Richmond, CA) using U16Sf 5′-AGAGTTTGATCCTGGCTCAG-3′ (SEQ ID NO: 20) and U16Sr R, 5′-CTTGTGCGGGCCCCCGTCAATTC-3′ (SEQ ID NOL 21).

The sequences were then searched against the NCBI nucleotide collection (nr/nt) database using the BLAST algorithm. Altschul et al. J. Mol. Biol. (1990) 215 (3): 403-10.

Selected isolates were identified with colony PCR using universal bacterial primer U16Sf and U16Sr in a 25 μL master mix consisting of 12.5 μL NEB Phusion master mix (NEB) and 2.5 L 10 UM primer mix. These PCR products were submitted to an outside partner (ACGT; Wheeling, IL) for sequencing using U16Sr, and identified using BLAST analysis against NCBI 16S rRNA species database. Altschul et al. J. Mol. Biol. (1990) 215 (3): 403-10.

### Antimicrobial Susceptibility Profiling

Candidates were sent to Microbial Research Inc. (Fort Collins, CO) for antimicrobial susceptibility analysis, performed as previously described (Susanti et al. Front Microbiol. (2021) 12:747845; Latorre et al. Front Vet. Sci. (2016) 3:95) for Bacillus. Lactobacillus was also analyzed at Microbial Research Inc. using broth microdilution method in laked horse blood (LHB) medium [Mueller Hinton broth (BD) containing 5% horse blood] following Clinical and Laboratory Standards Institute (CLSI) guidelines. Two-fold dilutions of the clinically relevant antibiotics (Clindamycin, Chloramphenicol, Erythromycin, Gentamicin, Kanamycin, Streptomycin, Tetracycline and Ampicillin; Sigma Aldrich; St. Louis, MO) were prepared in LHB medium. Approximately 50 μL of 1×10⁵ CFUs/mL of the Lactobacillus cells were added into each well. “No antibiotic” and “medium” alone controls were included. Escherichia coli ATCC 25923, Pseudomonas aeruginosa ATCC 27853, Staphylococcus aureus ATCC 29213, Enterococcus faecalis ATCC 29212, and Streptococcus pneumonia ATCC 49619 were used as quality control organisms. The Lactobacillus plates were incubated for 24-48 hours under microaerophilic conditions and Bacillus plates were incubated aerobically. Minimum inhibitory concentration (MIC) was defined as the lowest concentration of antibiotic that showed complete inhibition of candidate growth. The strains were classified as susceptible or resistant using the microbiological cut offs established by EFSA. Rychen et al. EFSA Journal (2018) 16 (4): e05206.

### Example 2

### Isolation of Genomic DNA

Genomic DNA for Illumina sequencing was isolated using the DNeasy blood and tissue kit (Qiagen; Hilden, Germany) for Gram-positive bacteria. Briefly, Lactilactobacillus strains were grown in MRS broth overnight under aerobic conditions for 14-16 hours without shaking. The cells were pelleted by centrifugation at 4,000×g for 10 minutes at 4° C. The pellet was washed once in 1 ml of PBS buffer (Invitrogen) and resuspended in 0.2 mL P1 buffer containing 100 μg/ml. RNase (Qiagen) and 6.25 mg/ml of lysozyme (Sigma Aldrich) and incubated at 37° C. overnight. After incubation, 20 μL of proteinase K (Qiagen) was added, mixed several times, and incubated at 55° C. for 1 hour. Subsequently DNA was purified without modification to the supplier protocol until elution in 100 μL distilled H₂O. Isolated DNA quantity was analyzed using Qubit 3.0 (Invitrogen) and integrity was confirmed by agarose gel electrophoresis.

### Whole Genome Sequencing (WGS)

Lactilactobacillus (see Table 1 above) whole genome sequencing (WGS) was performed using the Illumina platform.

Library preparations were performed according to the manufacturer's instructions for the Nextera DNA Flex Library Prep kit (Illumina; San Diego, CA). The concentration of DNA was confirmed using HS DNA Assay kit with the Qubit 3.0 (Invitrogen) and 300 ng of genomicDNA underwent the tagmentation process by enzymatic fragmentation, then sequence-specific overhangs attached using Bead-Linked Transposome technology. Following tagmentation, the samples were amplified with 5 cycles of PCR, using index labelled primers specific to the inserted sequences. Fragments were separated by size exclusion using SPRI-beads to obtain fragment sizes of about 600 base pairs. The eluted libraries were then confirmed for size and quality using the 4200 TapeStation High Sensitivity D1000 reagents (Agilent Technologies; Santa Clara, CA) and concentrations determined using Qubit HS DNA Assay (Invitrogen). Each library was diluted to a 4 nM stock and 5 μL of each library combined into a pooled library. The pooled library was then denatured by incubating with 0.2 N NaOH at room temperature for 5 minutes and diluted to a final concentration of 12 pM. The diluted pooled libraries were then added to the reagent cartridge (MiSeq Reagent Kit v3, Illumina) and analyzed using the MiSeq.

The genomes of L. curvatus strains PTA-127116 and PTA-127117, referred to herein as as PTA-16 and PTA-17 were further sequenced using PacBio platform. Bacterial pellet samples (Table 3) were sent to DNA Link, Inc (San Diego, CA) for WGS using PacBio RSII platform (PacBio; Menlo Park, CA).

TABLE 3
List of strains analyzed in the study.

| Study | | | | Source |
|---|---|---|---|---|
| identifier | Name | Genus | Species | organism |
| PTA-16 | L. curvatus ATCC | Lactilacto- | Curvatus | Atlantic |
| | PTA-127116 | bacillus | | salmon |
| PTA-17 | L. curvatus ATCC | Lactilacto- | Curvatus | Atlantic |
| | PTA-127117 | bacillus | | salmon |

The L. curvatus species listed in Table 3 were deposited in the American Type Culture Collection, located at 10801 University Boulevard, Manassas, Va., 20110-2209, USA.

Briefly, 20 kb DNA fragments were generated by shearing genomic DNA using the Covaris G-tube according to the manufacturer's recommended protocol (Covaris; Woburn, MA). Smaller fragments were purified by the AMpureXP bead purification system (Beckman Coulter; Brea, CA), For library preparation, 5 μg of genomic DNA was used. The SMRTbell library was constructed using SMRTbell™ Template Prep Kit 1.0 (PacBio®). Small fragments were removed using the BluePippin Size selection system (Sage Science; Beverly, MA). The remaining DNA sample was used for large-insert library preparation. A sequencing primer was annealed to the SMRTbell template and DNA polymerase was bound to the complex using DNA/Polymerase Binding kit P6 (PacBio®). Following the polymerase binding reaction, the MagBead was bound to the library complex with MagBeads Kit (PacBio®). This polymerase-SMRTbell-adaptor complex was loaded into zero-mode waveguides. The SMRTbell library was sequenced by 2 PacBio® SMRT cells (PacBio®) using the DNA sequencing kit 4.0 with C4 chemistry (PacBio®). A 1×240-minute movie was captured for each SMRT cell using the PacBio® RS sequencing platform. The genome was further assembled by DNA link, Inc with HGAP.3 protocol.

### Assembly of Illumina Sequence Data

Low quality reads trimming, and adaptor removal was performed using Trimmomatic software version 0.39. Bolger et al. Bioinformatics (2014) 30 (15): 2114-20. Paired end reads for 18 Lactobacillus samples were filtered using leading, trailing window of 20 and sliding window of 5 with average quality score of 20 to retain high quality reads. High-quality reads were used for de novo genome assembly with Unicycler (Wick et al. PLoS Comput Biol. (2017) 13 (6): e1005595) using the default assembly method. Scaffolds were filtered for a minimum of 200-bp read length. The quality of the subsequent assemblies was assessed by mapping the reads to assembly using bwa. Li H. Aligning sequence reads, clone sequences and assembly contigs with BWA-MEM. arXiv pre-print server. 2013. Genome completeness was found to be 99.46% for all the strains, using the CheckM lineage. Parks et al. Genome Res. (2015) 25 (7): 1043-55 Genome annotation, and feature prediction

Genome annotation was carried out using NCBI prokaryotic genome annotation pipeline, Prokaryotic Genome Annotation Pipeline (PGAP) that combines alignment based methods with methods of predicting protein-coding and RNA genes and other functional elements directly from sequence. Tatusova et al. Nucleic Acids Res. (2016) 44 (14): 6614-24. The biosynthetic gene clusters for secondary metabolites were determined using Antismash 5.0. Blin et al. Nucleic Acids Res. (2019) 47 (W1): W81-W7.

### Data Deposition

The raw sequencing reads, genome assemblies and annotations in this study were deposited in the NCBI BioProject following genome and bioproject accession numbers (Table 1 above).

### Example 3 Phylogenetic

### Analyses

Phylogenetic relationships of the genomes were explored with UBCG v3.0 using default settings. Na et al. J Microbiol. (2018) 56 (4): 280-5. This software tool employs a set of 92 single-copy core genes commonly present in all bacterial genomes. These genes then were aligned and concatenated within UBCG using default parameters. The estimation of robustness of the nodes is done through the gene support index (GSI), defined as the number of individual gene trees, out of the total genes used, that present the same node. A maximum-likelihood phylogenetic tree was inferred using FastTree v.2.1.10 with the GTR+CAT model. Price et al. PLoS One (2010) 5 (3): e9490.

### Identification of Prophages, Transposases and Other Insertion Sequences (IS)

Insertion sequence prediction was done using ISEscan v.1.7.2.1 (48). Prophage prediction was done using PhiSpy v4.2.6 which combines similarity- and composition-based strategies. Akhter et al. Nucleic Acids Res. (2012) 40 (16): e126.

### Example 4

### Probiotic and Treated Feed Preparation

Lactilactobacillus spp. were cultured for in vivo testing in BioStat B-DCU fermenters (Sartorius; Göttingen Germany) using MRS broth (BD). Cultures were dried in LyoStar 3 lyophilizer (SP; Warminster, PA), then lyophilized cake was powdered using mortar and pestle. Bacillus spp. were cultured from single colonies in sporulation medium ([8 g Bacto nutrient broth, 1 g KCl, 0.12 g MgSO₄·7H₂O, 5 g dextrose]/L adjusted to pH 7.6 with NaOH, with 0.1% each 1 M CaCl₂), 0.01 M MnSO₄, and 1 mM FeSO₄) for 96 hours, then washed and resuspended in cold PBS (Invitrogen). Maltodextrin solution was added for a final concentration of 15%, and spores were spray dried in a Buchi mini spray dryer (Buchi, Flawil, Switzerland) at an outlet temperature of 104° C. Dried spores with maltodextrin were mixed with 1.5% calcium phosphate as a desiccant.

Five groups were tested in the improvement of growth performance. 10 kg commercial extruded feed pellets were top coated with 750 g IVP. This IVP was prepared based on colony forming units (CFU) from 3-8% lyophilized bacteria or 0.01-0.2% spray dried spores, 91-99% food-grade excipients. Feed was coated first with IVP, then with 1% fish oil, then mixed for one additional minute. This process was repeated on larger feed pellets when fish reached 60 g. After final drying pellets were stored in plastic bags at 15° C.

### Performance Study

A 7-week study was performed. 600 Atlantic salmon parr weighing 30-50 g were recruited from internal populations, distributed without intentional bias in twelve 100 L study tanks randomly allocated to 6 groups with 2 replicate tanks for each group, and acclimatized to the conditions of the feeding trial and fed with a basal diet for seven days without handling. The control group (NCP) was fed commercial extruded basal diet and the probiotic group was given 75 mg/kg probiotic IVP corresponding to 2.23×10⁶-16×10⁸ CFUs/gram of feed. Feed caliber was adjusted according to biweekly sample weights. Fish were fed approximately 110% of the specific feed rate (SFR) using a Skretting feed table. Over the study period, fish were maintained in 100 L tanks of flow through fresh water under a photoperiod regime of 24-hour day light. Water flow during the holding period and during experimental period was set at a rate to ensure a minimum of 2.0 total volume water exchange/hour.

Supplemental oxygen was delivered as needed to the tank water to maintain appropriate saturated oxygen levels (70-130% saturation) and water temperature for all tanks was monitored daily.

### Global Untargeted Metabolomic Analysis

Study design: Two different probiotic strains of Lactilactobacillus were selected for this analysis and sent to MicroMGx (Chicago, IL) for analysis. Twelve different culture conditions were selected to influence the growth and metabolism of the Lactilactobacillus strains. The selected media additives are listed in Table 4, with concentrations listed.

TABLE 4
Media additive concentrations used in the study.

| Additive | Concentration | |
|---|---|---|
| Glucose | 111 | mM |
| Maltose | 58.5 | mM |
| Lactose | 58.5 | mM |
| L-fucose | 30 | mM |
| N-acetylglucosamine | 20 | mM |
| Inulin | 0.5% | w/v |
| Galactooligosaccharide | 0.5% | w/v |
| Galactooligosaccharide + zinc | 0.5 | mM |
| Galactooligosaccharide + vitamin d3 | 0.021 | mM |
| Galactooligosaccharide + ascorbic acid | 2.84 | mM |

| | |
|---|---|
| Galactooligosaccharide + zinc + vitamin | Above |
| d3 + ascorbic acid | concentrations |
| Bile salts | 0.3% |

To ensure statistical power, 3 biological replicates of each sample were analyzed. The total number of samples was 12 culture conditions×2 different strains×3 biological replicates=72 total samples. Additionally, each of the culture media were extracted and analyzed to enable the identification and removal of background signals. Samples were analyzed in random sequence to manage batch effects.

### Sample Preparation

Cultures of each of the Lactilactobacillus strains were grown overnight in MRS (BD) broth. Overnight cultures were then used to inoculate modified MRS broth (animal-origin peptones were replaced with vegetable proteose peptone; Sigma-Aldrich #29185) containing additives listed in the Table 4. Cultures were grown anaerobically for 72 hours. The cells and culture supernatant were separated by centrifugation 5 min at 16,000×g. Culture supernatant was extracted using Oasis HLB solid phase extraction cartridges (Waters; Milford, MA) and then dried down in a vacuum centrifuge for later use.

### Metabolomics Data Acquisition

Samples were analyzed on a Q-Exactive mass-spectrometer (Thermo Fisher) coupled to an Agilent 1200-series UHPLC.

### Identification of Metabolite Features

Metabolite features are defined as a specific m/z signal associated with a specific retention time. The features shown in this report were determined to be significant because they showed a change in abundance across media conditions of greater than two-fold, with a significance between groups (one-way ANOVA, *P<0.05). Where possible, metabolite features are assigned putative identifications by searching their observed accurate mass against a database of small molecules that are produced by bacteria.

### Lactilactobacillus Isolation and Molecular Identification

Of the 900 microbiome isolates cultured from Atlantic salmon intestine, 18 Lactilactobacillus isolates were cultured from Norwegian and North American salmon. Eight Lactilactobacillus strains including two strains described in the WGS and metabolomics portions of this study were isolated from the intestine of Atlantic salmon received from Norway. One Lactilactobacillus strain was isolated from hindgut and nine Lactilactobacillus strains were isolated from foregut of grower salmon from facilities in North America. 17 spore-forming Bacillus strains were isolated from the intestine of Atlantic salmon parr from a hatchery in Chile (Table 5).

TABLE 5
Description of probiotic library, Bacillus and Lactilactobacillus candidates.

| | | | | Bacillus | | Lactilactobacillus |
|---|---|---|---|---|---|---|
| Geographic | | Total | | listed | | listed |
| Source | Sample type | isolates | Bacillus | in QPS | Lactilactobacillus | in QPS |
| Norway | Parr and | 268 | 0 | 0 | 8 | 8 |
| | grower | | | | | |
| | intestine | | | | | |
| North | Grower | 394 | 0 | 0 | 10 | 10 |
| America | intestine | | | | | |
| Chile | Parr intestine | 238 | 17 | 6 | 0 | 0 |

Six of these Bacilli, and all of the Lactilactobacilli are listed in EFSA's qualified presumption of safety (QPS) list, suggesting they may be considered safe for probiotic use. Koutsoumanis et al. EFSA Journal (2020) 18 (2). Three of the Bacillus strains showed closest homology to B. velezensis, three showed closest homology to B. subtilis, identified by 16S sequencing and BLAST analyses (Table 6).

Based on the WGS and respective BLAST search comparison results, three Lactilactobacillus strains showed closest homology to L. sakei, and 15 of the strains, including PTA-16 and PTA-17, showed closest homology to published L. curvatus sequences (Table 6).

TABLE 6
Bacillus and Lactilactobacillus isolates and their growth profiles.

| | | | | Fish | | | |
|---|---|---|---|---|---|---|---|
| | | | | size | Microaerophilic | Aerobic | 15° C. |
| Strain | Geography | Sample | Water | (g) | growth | growth | Growth |
| BvELA005 | Chile | Intestine | Freshwater | 38 | ND | + | + |
| BvELA006 | Chile | Intestine | Freshwater | 38 | ND | + | + |
| BvELA014 | Chile | Intestine | Freshwater | 95 | ND | + | + |
| BsELA015 | Chile | Intestine | Freshwater | 95 | ND | + | + |
| BsELA016 | Chile | Intestine | Freshwater | 95 | ND | + | + |
| BSELA017 | Chile | Intestine | Freshwater | 95 | ND | + | + |
| LcELA23 | Norway | Intestine | Seawater | 1500 | + | + | + |
| LcELA29 | Norway | Intestine | Seawater | 1500 | + | + | + |
| LcELA33 | Norway | Intestine | Seawater | 1500 | + | + | + |
| LcELA92 | Norway | Intestine | Seawater | 1500 | + | + | + |
| PTA-16 | Norway | Intestine | Seawater | 1500 | + | + | + |
| LcELA96 | Norway | Intestine | Seawater | 1500 | + | + | + |
| LcELA98 | Norway | Intestine | Seawater | 1500 | + | + | + |
| PTA-17 | Norway | Intestine | Seawater | 1500 | + | + | + |
| LcELA2 | North | Hindgut | Seawater | 1200 | + | + | + |
| | America | | | | | | |
| LcELA59 | North | Foregut | Seawater | 1200 | + | + | + |
| | America | | | | | | |
| LcELA60 | North | Foregut | Seawater | 1200 | + | + | + |
| | America | | | | | | |
| LcELA61 | North | Foregut | Seawater | 1200 | + | + | + |
| | America | | | | | | |
| LcELA62 | North | Foregut | Seawater | 1200 | + | + | + |
| | America | | | | | | |
| LsELA64 | North | Foregut | Seawater | 6000 | + | + | + |
| | America | | | | | | |
| LsELA65 | North | Foregut | Seawater | 6000 | + | + | + |
| | America | | | | | | |
| LfELA68 | North | Foregut | Seawater | 6000 | + | + | + |
| | America | | | | | | |
| LcELA388 | North | Foregut | Seawater | 6000 | + | + | + |
| | America | | | | | | |
| LsELA391 | North | Foregut | Seawater | 6000 | + | + | + |
| | America | | | | | | |

Bv: Bacillus velezensis,
Bs: B. subtilis,
Lc: Lactilactobacillus curvatus,
Ls: L. sake,
Lf: L. fuchuensis

### Growth Profiles

All the Lactilactobacillus strains had similar growth profiles. All the 18 strains grew on MRS agar and broth microaerobically and aerobically, at 15° C. and 23° C. This is consistent with their isolation from cold water fish in water temperature 8.7-12° C. Bacillus candidates also grew at 15° C. (Table 6).

### Example 5

### Genomic Characterization

### In Silico Analysis

A total of 18 genomes were sequenced and characterized in this study. The genomes of PTA-16 and PTA-17 were sequenced by PacBio sequencing platform while the remaining strains were sequenced using Illumina platform. PTA-16 contained 3 contigs yielding a total estimated genome size of 1.99 Mb and PTA-17 contained 2 contigs yielding a total estimated genome size of 1.97 Mb. The genome properties, prediction and annotation of different features are summarized in (Table 7).

TABLE 7
Genomic properties of Lactilactobacillus strains.

| | | | | | | | | | Transfer | Non- | | |
|---|---|---|---|---|---|---|---|---|---|---|---|---|
| | | Depth of | | | Regulatory | Repeat | Ribosomal | Transfer | messenger | coding | Misc. | Misc. |
| Strain | Contigs | coverage | Genes | CDSs | elements | regions | RNAs | RNAs | RNAs | RNAs | binding | feature |
| PTA-16 | 3 | 152 | 2,029 | 1,941 | 5 | 3 | 18 | 67 | 1 | 2 | 7 | 3 |
| PTA-17 | 2 | 746 | 2,007 | 1,921 | 6 | 1 | 18 | 65 | 1 | 2 | 7 | 3 |
| LcELA388 | 2 | 347 | 2,133 | 2,047 | 6 | 4 | 18 | 65 | 1 | 2 | 8 | 3 |
| LsELA391 | 3 | 413 | 2,135 | 2,045 | 7 | 1 | 21 | 66 | 1 | 2 | 6 | 3 |
| LcELA2 | 78 | 801 | 1,885 | 1,835 | 5 | 3 | 3 | 44 | 1 | 2 | 7 | 3 |
| LcELA23 | 88 | 689 | 1,922 | 1,867 | 6 | 1 | 3 | 49 | 1 | 2 | 7 | 3 |
| LcELA29 | 79 | 364 | 1,913 | 1,863 | 6 | 1 | 3 | 44 | 1 | 2 | 7 | 3 |
| LcELA33 | 94 | 794 | 1,897 | 1,842 | 5 | 3 | 3 | 49 | 1 | 2 | 7 | 3 |
| LcELA59 | 71 | 300 | 1,770 | 1,721 | 6 | 2 | 3 | 44 | 1 | 2 | 7 | 3 |
| LcELA60 | 67 | 347 | 1,778 | 1,728 | 6 | 2 | 3 | 44 | 1 | 2 | 7 | 3 |
| LcELA61 | 66 | 304 | 1,872 | 1,820 | 6 | 2 | 3 | 46 | 1 | 2 | 7 | 3 |
| LcELA62 | 69 | 304 | 1,809 | 1,761 | 6 | 1 | 2 | 44 | 1 | 2 | 7 | 3 |
| LcELA92 | 79 | 598 | 1,879 | 1,829 | 5 | 3 | 3 | 44 | 1 | 2 | 7 | 3 |
| LcELALA96 | 79 | 731 | 1,890 | 1,840 | 5 | 3 | 3 | 44 | 1 | 2 | 7 | 3 |
| LcELA98 | 76 | 877 | 1,914 | 1,865 | 6 | 1 | 2 | 44 | 1 | 2 | 7 | 3 |
| LfELA68 | 45 | 337 | 1,842 | 1,816 | 6 | 1 | | 23 | 1 | 2 | 6 | 3 |
| LsELA64 | 41 | 229 | 2,089 | 2,032 | 7 | 3 | 2 | 52 | 1 | 2 | 6 | 3 |
| LsELA65 | 22 | 419 | 1916 | 1883 | 7 | | | 30 | 1 | 2 | 8 | 3 |

### Phylogenetic Analysis

Phylogenetic relationships of the genomes were explored with UBCG v3.0, which employs a set of 92 single-copy core genes commonly present in all bacterial genomes. These genes then were aligned and concatenated within UBCG using default parameters. The estimation of robustness of the nodes is done through the gene support index (GSI), defined as the number of individual gene trees, out of the total genes used, that present the same node.

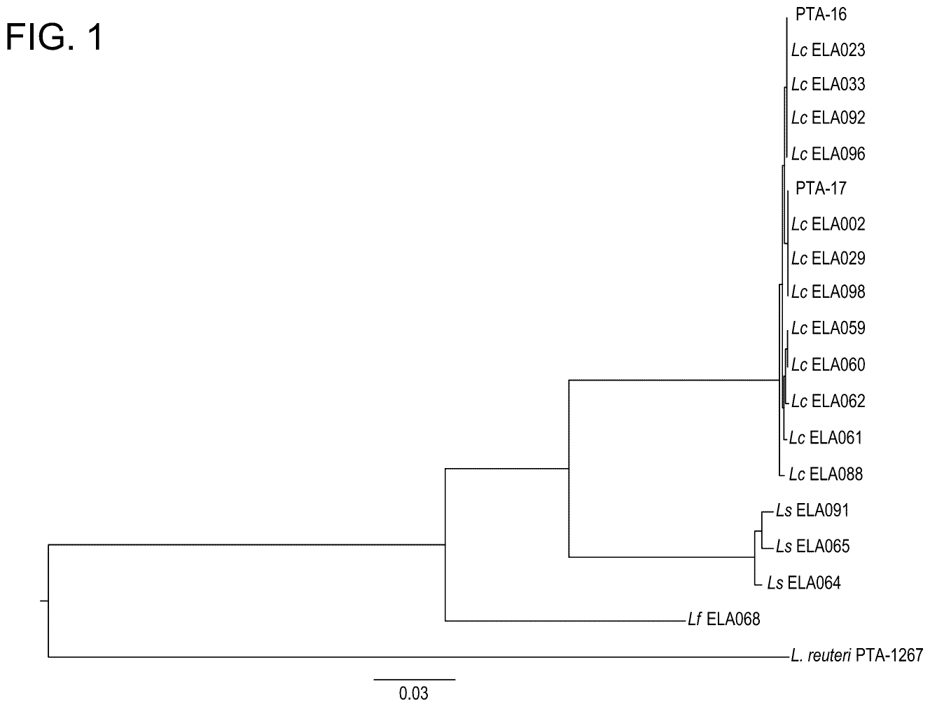

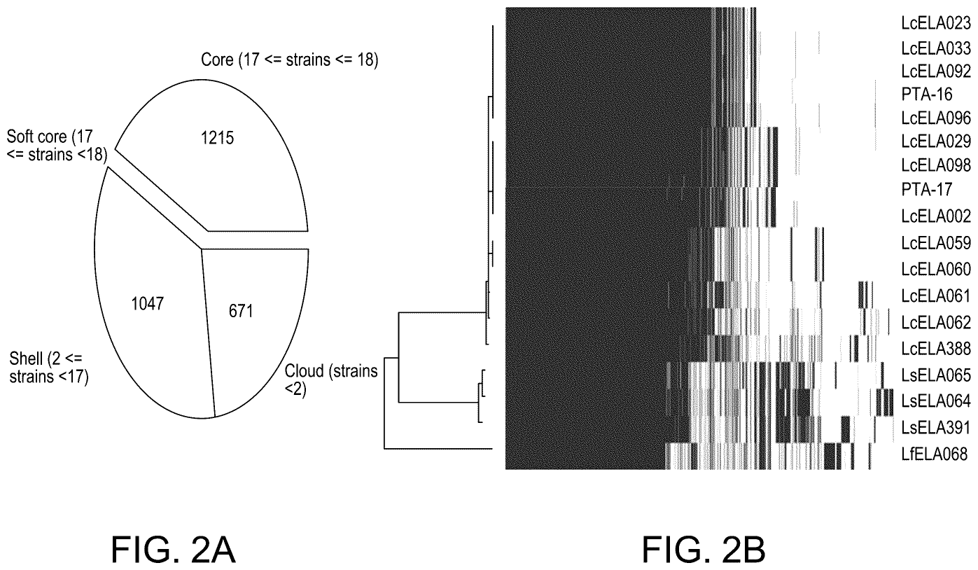

Phylogenetic analysis was performed on 18 Lactobacillus strains with L. reuteri strain ATCC PTA-126788 as an outgroup. As shown in FIG. 1, different Lactobacilli neatly grouped into their respective species clades.

### Comparative Genomics Analyses

Ortholog analysis was performed to identify paralogous and/or orthologous relationships between genomes of L. curvatus, L. sakei and L. fuchuensis strains. Overall, 98.6% of the genes were shared between strains with 1215 orthogroups having membership of at least one gene in all 18 genomes. L. curvatus strains PTA-16 and PTA-17 shared 1681 and 1663 genes among them, respectively. Orthology-based multi-protein phylogenetic tree was used to identify optimal strain combinations from different clades.

### Screening for Prophages, ISs and Transposases

Genomes were scanned for the presence of mobile genetic elements such as prophages, insertion sequences (ISs) and transposases. Both PTA-16 and PTA-17 strains contained seven prophage regions each. However, there were 3 phage genes (all coding for Tyrosine recombinase protein) in both genomes that were outside of prophage regions. Putative IS and associated proteins predicted by ISEscan revealed 79 ORFs in 10 IS families in strain PTA-16 and 68 ORFs in 10 IS families in strain PTA-17.

### Absence of Virulence Factors and Toxins

Both PTA-16 (3 contigs) and PTA-17 (2 contigs) strains were confirmed to be free of known virulence factors and/or toxins by comparing against virulence factor database (VFDB; search parameters of 380% identity and 380% alignment length/coverage), which is an integrated comprehensive online resource database for curating information about bacterial virulence factors and/or toxins. All other genomes were also free of virulence factors and toxins.

### Absence of Acquired Antimicrobial Resistance Genes

The genomes of PTA-16 and PTA-17 along with other Lactilactobacillus strains were searched for potential antimicrobial resistance genes against multiple AMR databases including NCBI-AMR, Resfinder DB and ARG-ANNOT using Abricate. The screening did not identify any potential antimicrobial resistance genes in any of the genomes except for LcELA65. LcELA65 contained a gene encoding tetracycline-resistant ribosomal protection protein (tetW) that confers resistance to tetracycline.

### Screening for Genes Involved in Biogenic Amines and Toxins

Functional annotation of the PTA-16 and PTA-17 genomes revealed that these strains do not contain any known protein-encoding genes involved in the production of biogenic amines with the exception of ornithine decarboxylase in the genome of PTA-16. Interestingly, both genomes had incomplete CDSs encoding tyrosine decarboxylase. No other toxins were identified in the genomes of both strains.

### Genes Involved in the Production of Lactic Acid and Other Beneficial Metabolites

Both PTA-16 and PTA-17 strains contained one CDS encoding for L-lactate dehydrogenase. However, no CDS encoding for D-lactate dehydrogenase (EC 1.1.1.28) was found in any of the sequenced strains.

Several coding sequences involved in adhesion of Lactobacilli to intestinal epithelium including chaperonin GroEL, signal peptidase II and elongation factor Tu were identified in both PTA-16 and PTA-17 genomes. Search for desired stress tolerance features in PTA-16 and PTA-17 strains revealed the presence of three CDSs encoding for DNA protection during starvation. Another stress resistant gene putatively encoding for phosphate starvation-inducible PhoH-like protein was also found in both strains.

### Example 6 Antimicrobial Susceptibility

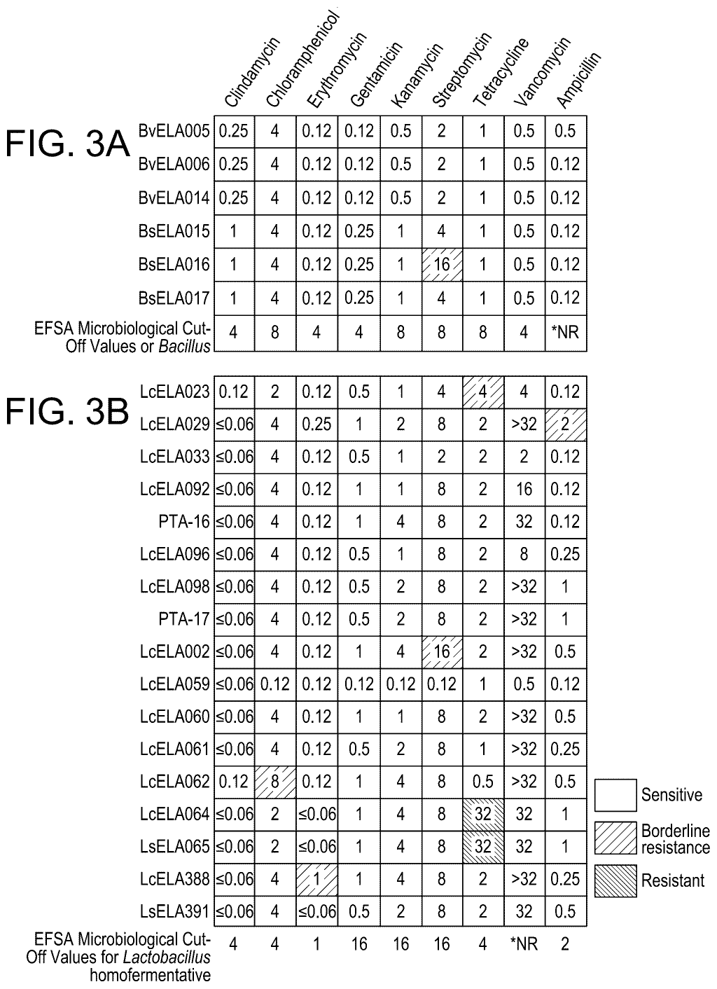

Minimum inhibitory concentrations were analyzed against relevant antibiotics according to EFSA guidelines (Rychen et al. EFSA Journal (2018) 16 (4): e05206), including Ampicillin, Vancomycin, Gentamicin, Kanamycin, Streptomycin, Erythromycin, Clindamycin, Tetracycline and Chloramphenicol. BvELA005, BvELA006, BvELA014, BsELA015, and BsELA017 were determined to be sensitive to all relevant antibiotics according to EFSA guidelines, while BsELA016 was sensitive to all relevant antibiotics except streptomycin, which was a two-fold dilution above the EFSA cut-off. BvELA005, BvELA006, BvELA014, BsELA015, BsELA017, LcELA33, LcELA92, PTA-16, LcELA96, LCELA98, PTA-17, LcELA59, LCELA60, LCELA61, and LsELA391 strains were sensitive to all relevant tested antibiotics according to EFSA guidelines (Rychen et al. EFSA Journal (2018) 16 (4): e05206), with MIC values at or below the reported species characteristic cut-off values (FIG. 3). BsELA16 and LcELA2 were one- or two-fold dilutions above EFSA microbiological cutoff of streptomycin, and LcELA23, LcELA29, LcELA62, and LcELA388 were one or two fold dilutions above cut off value of tetracycline, ampicillin, chloramphenicol, and erythromycin, respectively. This is considered acceptable due to the technical variation of the phenotypic method as recognized previously. NCCLS. Development 578 of in vitro susceptibility testing criteria and quality control parameters; approved 579 guideline 2nd ed. Wayne, PA, USA.: NCCLS documents M23-A2 NCCLS; 2001. LfELA68 was not viable in any MIC medium tested; LsELA64 and LsELA065 were highly resistant to tetracycline which rules them out as probiotic candidates.

### Example 7

### In Vivo Efficacy

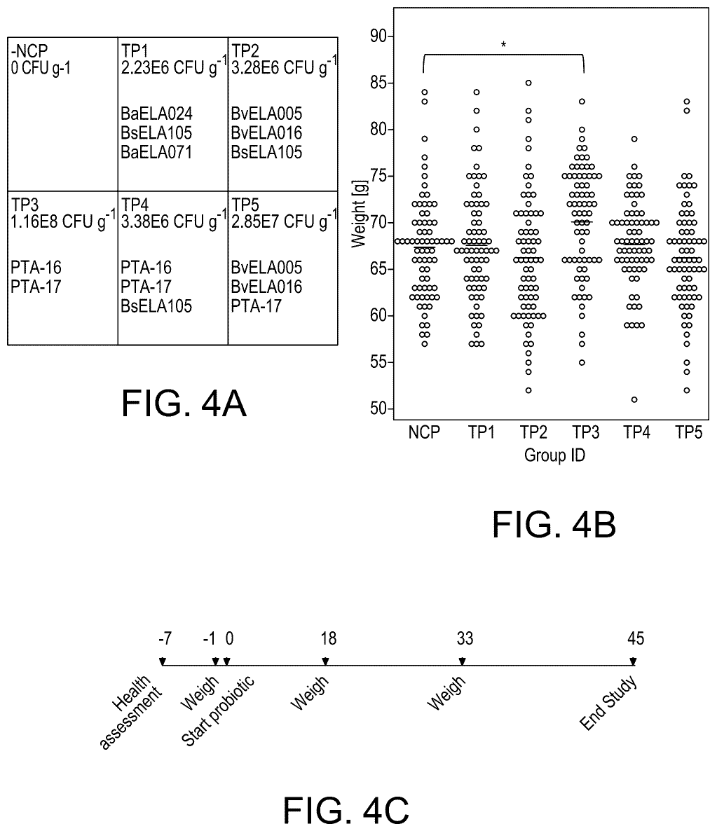

Five test products and one negative control product (FIG. 4A) were added to commercial fish pellets and fed to six groups of 100 fish each divided between two tanks per group. 10 fish were randomly selected from each tank, weighed, and returned on study days (SD)-1, 18, and 33. 70 fish were weighed, and all euthanized at the end of the study on SD 45 (FIG. 4B). While TP1 (Test Product 1), TP2, TP4, and TP5 weights did not differ significantly from NCP, TP3 weighed significantly more (p<0.01, n=70). Average weights for TP1, TP2, TP3, TP4, TP5, and NCP respectively were 67.61 g, 62.40 g, 70.23 g, 67.79 g, 66.56 g, and 67.37 g. Weights relative to the control group were 0.4%, −1.7%, 4.2%, 0.6%, and −1.7% for TP1, TP2, TP3, TP4, and TP5, respectively. No gross pathology (data not shown) nor mortality revealed concerns about safety, and the study timeline is shown in FIG. 4C.

### Global Untargeted Metabolomic Analysis

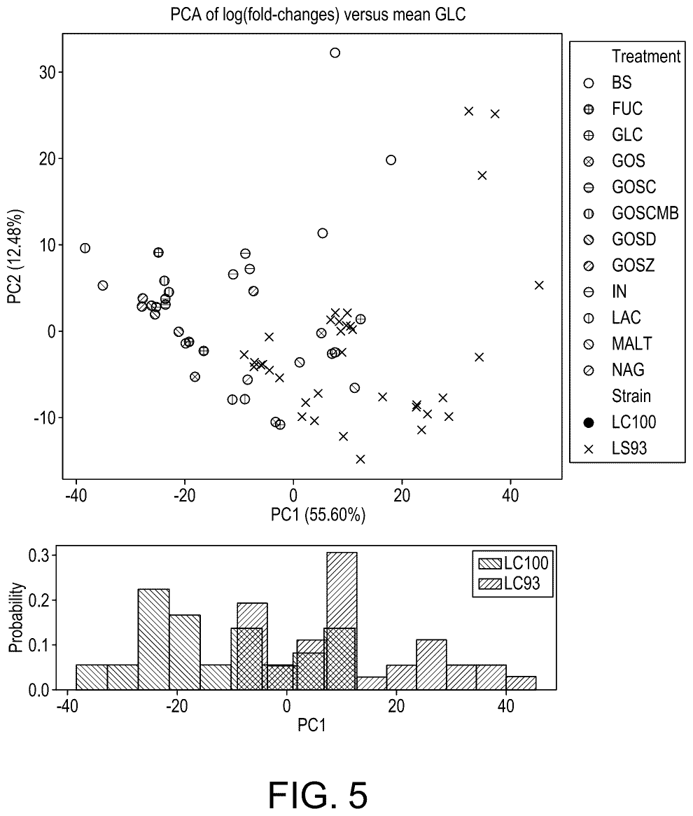

In order to compare the responses of both strains to different media additives, we compared their metabolomics profiles under different growth conditions. For the comparison, we carried out a principal component analysis of the log₂ fold-changes of feature abundances in each of the treatments compared to their average levels when the cells were grown on glucose. We only considered MS features present across all conditions with at minimum a 2-fold change in abundance compared to the glucose control in at least one sample (192 metabolites in total). As observed in FIG. 5, metabolomics profiles clustered by strain along the first principal component, representing over 50% of the variance in feature changes across strains, growth conditions and technical replicates. While media additives such as N-acetyl-glucosamine, and galactooligosaccharides (GOS) resulted in minor differences compared to the glucose control, additives including lactose, inulin, and GOS amended with vitamins and zinc resulted in larger metabolite shifts. Notably, the addition of bile salts resulted in distinct metabolomic profiles explaining most of the variance along the second principal component and clustering the samples of both strains.

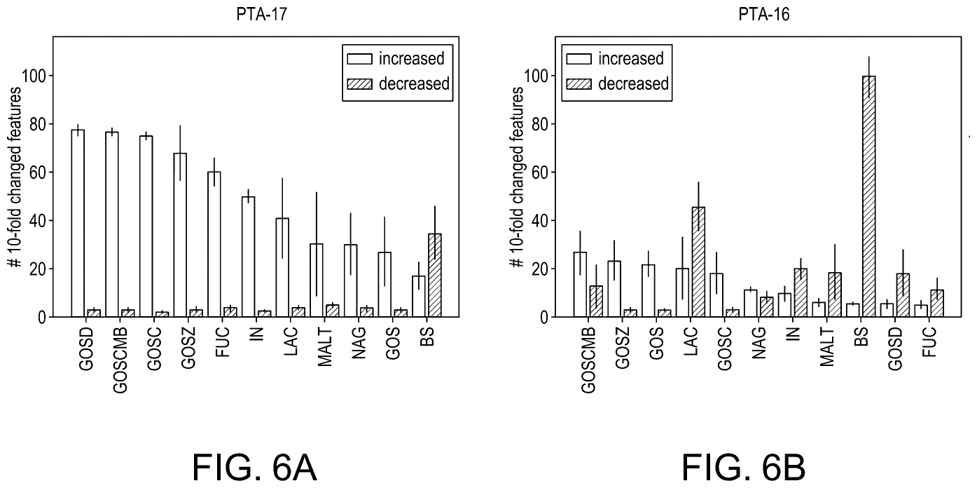

Comparing the magnitude of feature abundance changes showed that the different media additives caused more features to increase in abundance by more than ten-fold in PTA-17 than PTA-16 when compared to a glucose control (FIG. 6). Many of the prebiotic ingredients tested resulted in more than twice the number of features with increased expression in PTA-17.

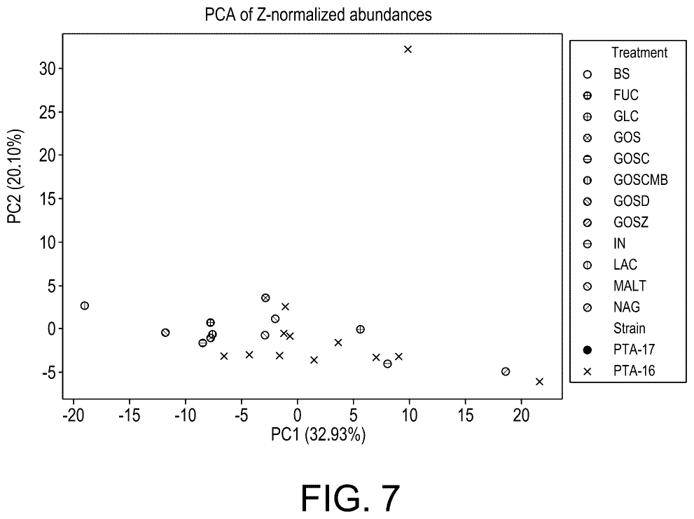

Looking at the abundances of individual features across media additives instead of the changes relative to a control media showed less clustering of samples by strain (FIG. 7). So, while the overall composition of both strains for the features analyzed is similar across conditions, strains respond differently to distinct additives. Consistent with the above results, samples from both strains supplemented with bile acids cluster together and away from the remaining treatments. Additionally, addition of lactose leads to the highest divergence in metabolomics profiles between both strains.

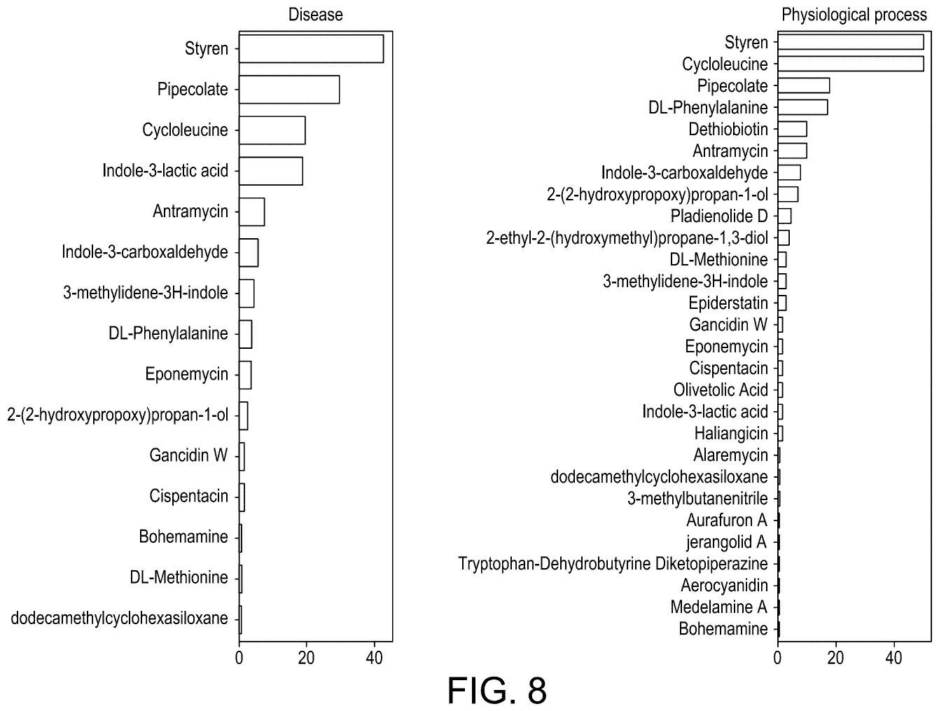

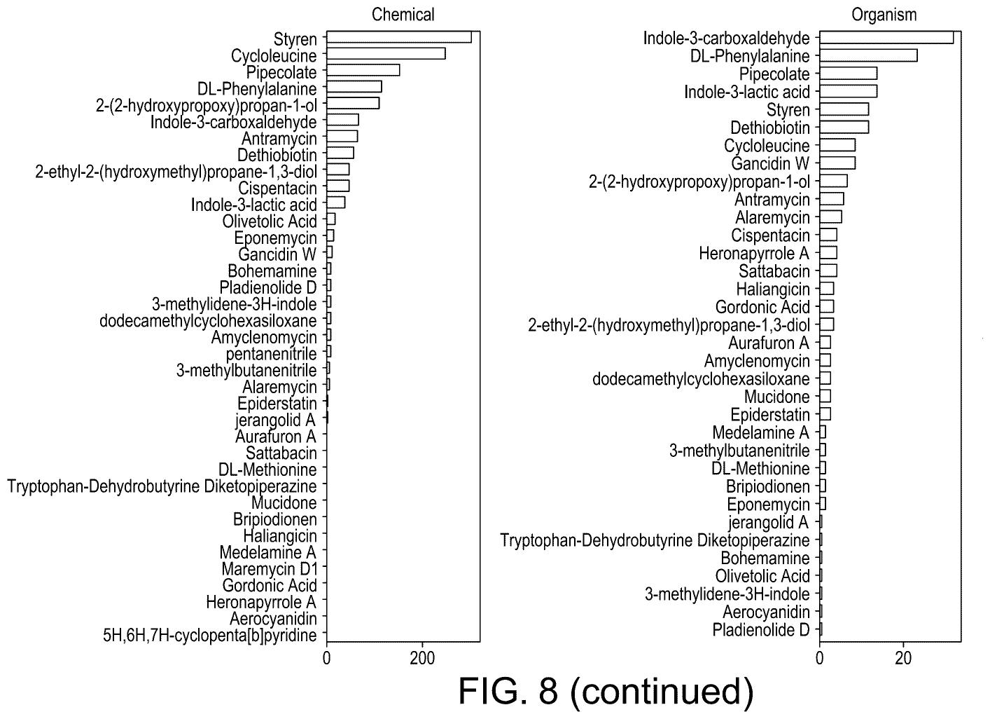

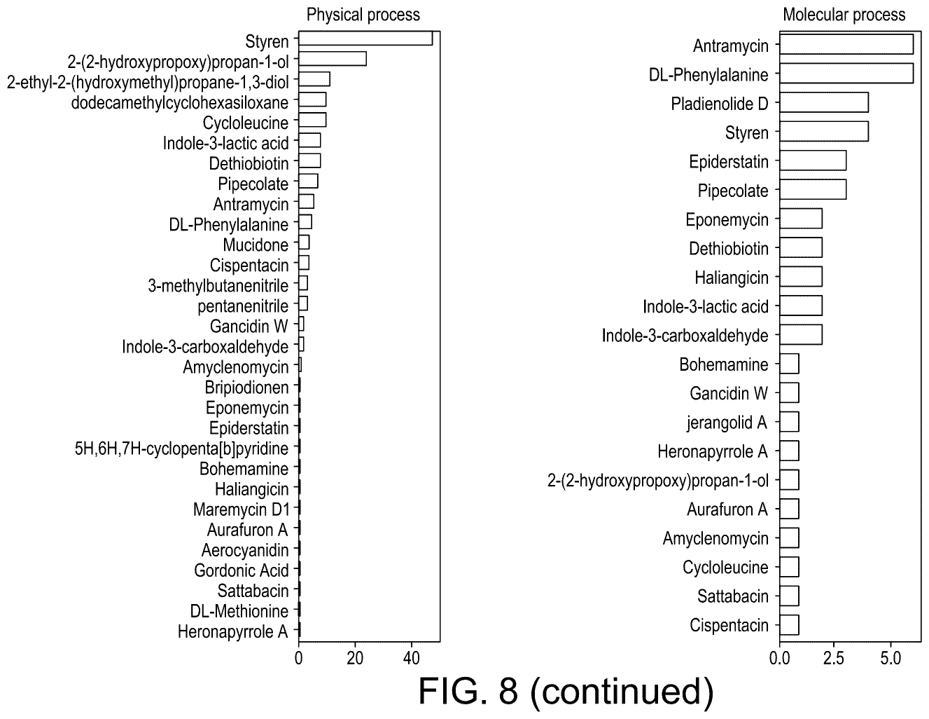

Out of the recovered MeSH terms associated with potential metabolites produced by the strains, 46% corresponded to chemicals, 10% to diseases, 6% to physical processes and 5%, to living organisms (FIG. 8). For example, multiple compounds (Aurafuron, Antramycin, Pladienolide, Epiderstatin, Eponemycin, Gancidin, and Medelamine) were associated with antibiosis, and metabolites including cycloleucine and 3-Methyleneindolenine were associated with body weight.

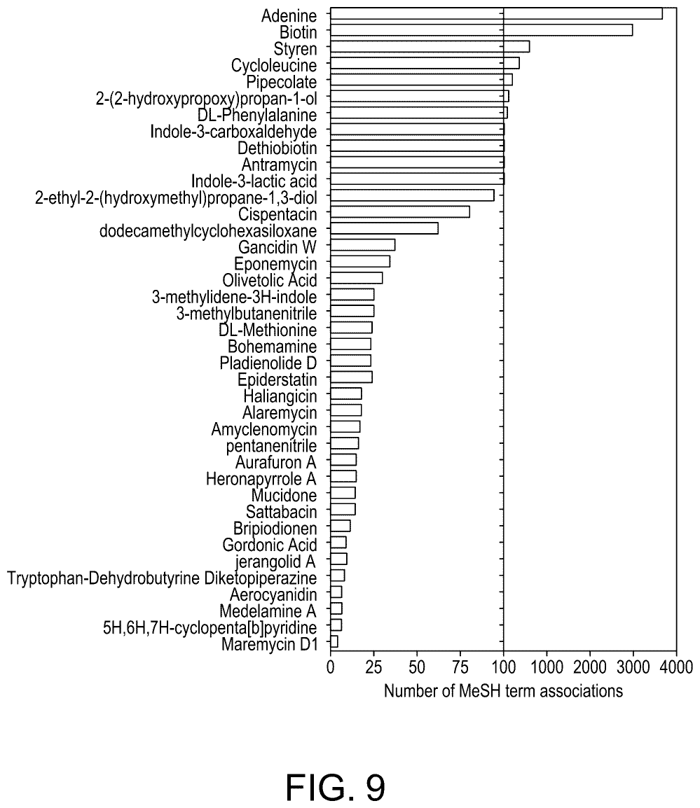

Out of about 200 features analyzed, 136 could be mapped to 5 or less potential identities in the MicroMGX database and none were uniquely mapped (Data not shown). In order to gain a broad idea of the possible physiological roles of these molecules we followed the approach outlined by Sartor et al. (PMID: 22492643) to identify medical subject headings (MeSH terms) associated with metabolites detected in cultures of LC100 and LS93 based on their co-occurrence across published research. The inventors identified at least one MeSH term associated with 39 out of 179 potential metabolite identities of MS features, representing 10005 significant associations (FDR<0.05) to 6239 MeSH terms (FIG. 9). These relationships illustrate the extent to which the identified compounds have been previously discussed in the scientific literature. Most of the associations uncovered were accounted for by Adenine and Biotin, whose central metabolic roles have been extensively studied. For the remaining metabolites, between 4 and 725 associations were identified.

With the goal to isolate and develop endogenous microbial isolates as potential probiotics to improve weight gain and enhance disease resistance in salmon, samples were collected from various growth stages (parr, smolts and grower) and major fish production sites (Norway, Chile, and North America). Smejkal G B, Kakumanu S. Safely meeting global salmon demand. npj Science of Food. 2018; 2 (1); rsen A, Asche F, Hermansen Ø, Nystøyl R. Production cost and competitiveness in major salmon farming countries 2003-2018. Aquaculture. 2020; 522:735089.

Parr and smolts were raised at 11-12° C. in freshwater while growers were raised at 8.7-12° C. in seawater. A library of 900 bacterial isolates were cultured from the intestines, skin, and gills of farmed Atlantic salmon. 16S rRNA sequencing identified 623 of these organisms, which informed selection of probiotic candidates from promising genera, sample diversity, and regulatory lists. Carnobacterium, Alivibrio and Lactobacillus were among the top probiotic genera isolated from Norwegian and North American samples. Carnobacteria are lactic acid bacteria which dominate fish hindgut by population; and non-pathogenic strains of Carnobacteria have been previously shown to improve weight gain and disease resistance in farmed Atlantic cod and salmon. Similarly, bathing with Alivibrio strains improves growth and FCR, and reduces mortality in Atlantic salmon. Spore-forming Bacillus are not a major part of the endogenous microbes of salmon; in agreement with this, strains belonging to Bacillus were only isolated from Chilean salmon samples but not from Norwegian and North American salmon samples.

Owing to their proven health benefits and long history of safe use, Lactobacilli are among one of the most commonly used probiotics in both human and animal health and are increasingly being evaluated as potential probiotics for fish.

With the goal to develop native Lactobacilli from salmon gut as Direct Fed Microbials (DFMs), the inventors chose isolates belonging to Lactobacillus species from foregut and hindgut samples that are listed in qualified presumption of safety (QPS) list put forth by the European Food Safety Authority for further characterization. Koutsoumanis K, Allende A, Alvarez-Ordóñez A, Bolton D, Bover-Cid S, Chemaly M, et al. Update of the list of QPS-recommended biological agents intentionally added to food or feed as notified to EFSA 11: suitability of taxonomic units notified to EFSA until September 2019. EFSA Journal. 2020; 18 (2). Indeed, all Lactobacillus strains isolated from salmon intestine were identified by 16S rRNA sequencing as members of the QPS list. Despite the long-established potential for lactic acid bacterial probiotics, only one product containing Pediococcus acidilactici is commercially available. The L. curvatus strains PTA-16 and PTA-17 described herein may be used as probiotics in Atlantic salmon.

Whole genome sequencing of Lactilactobacillus isolates allowed species identity confirmation from 16S sequencing, revealing 15 L. curvatus and three L. sakei isolates, confirming their place on the QPS list. In the initial round of strain characterization, we used Illumina platform to sequence and assemble the genomes of all 18 strains. Phylogenetic analysis has previously divided L. curvatus by its ability to metabolize plant-derived carbohydrates, so PTA-16 and PTA-17 were selected from diverse phylogenetic groups and fish specimens to form consortia for the in vivo study. PTA16 and PTA17 were further sequenced using PacBio sequencing platform. Long read sequencing technology enabled complete genome characterization with each genome represented by large, nearly complete contigs.

Comprehensive functional annotation of the L. curvatus strains PTA-16 and PTA-17 revealed presence of several genes important for probiotic efficacy. Probiotic bacteria are known to contain bioactive secondary metabolites that interact with other pathogenic bacteria to attenuate virulence.

Neither of the selected candidates seem to possess any bacteriocins found in Enzybase nor AntiSMASH. Analysis for antibiotic resistance genes revealed no hits using ResFinder, supporting PTA-16 and PTA-17 as safe probiotic candidates. Both PTA-16 and PTA-17 strains contained one coding sequence encoding L-lactate dehydrogenase (EC 1.1.1.27), which is responsible for lactic acid production. CDS encoding D-lactate dehydrogenase (EC 1.1.1.28) was not found in any of the strains. While the diversity of phages in gut ecosystems is getting increasingly well-characterized, knowledge is limited on how phages contribute to the evolution and ecology of their host bacteria. Prophage analysis of PTA-16 and PTA-17 showed 7 prophage regions each. Prophages can be advantageous for gut symbionts like L. curvatus by increasing its competitiveness in the intestinal niche.

Without being bound to a particular theory, the inventors found that the probiotics described herein are more effective than terrestrial probiotics, due to their adaptation to fish physiology and specific salinity and temperature requirements. While Bacillus probiotics have shown promising growth improvement in salmonids and other fish, the inventors found that their study revealed no improvement in terrestrial probiotics, nor in native Bacillus candidates, but only in native Lactilactobacillus candidates. As diadramous fish, salmon live in both freshwater and seawater. Lactobacillus dominate the gut of saltwater salmon compared with freshwater fish, and they are generally not recovered from very early stages. While these candidates were examined in available freshwater, growth in the Lactilactobacillus-fed group was significant improved at the end of the relatively short study. Lactilactobacillus probiotic's growth-enhancing effect may be amplified in longer freshwater as well as seawater environments.

Based on this in vivo performance improvement, PTA-16 and PTA-17 were further analyzed for their ability to secrete various metabolites in the first comprehensive study in the presence of different prebiotic additives. Synbiotics are the synergistic combination of prebiotic with probiotics, and since they have been shown to be beneficial in Caspian salmon, the inventors sought to identify potential prebiotics to enhance the efficacy of two L. curvatus candidates. Metabolomics revealed that when 11 prebiotics added to culture media, at least ten-fold PTA-16 and PTA-17 features were up- or down-regulated. This suggests that a synbiotic combination of the top probiotic candidates with one or more of these prebiotics is a promising approach to improve salmon performance.

For PTA-17, features are especially increased in the presence of inulin, GOS supplemented with vitamin D, vitamin C, zinc, and all three. Vitamin C and zinc have already been studied for supplementation for Atlantic salmon health so its inclusion with fish feed would be accessible and familiar to farmers. Inulin and GOS are popular, widely available prebiotics.

Features are especially decreased in the presence of bile salts, reflecting expected probiotic/digestive system interplay. For PTA-16, features are especially decreased in the presence of lactose and bile salts. The differences in feature upregulation between species in the presence of GOS is predicted by previous work on Lactobacillus galactooligosaccharide metabolism. These prebiotics could potentially work synergistically with PTA-16 and PTA-17 and enhance weight gain.

The inventors showed comprehensive genomic and promising in vivo evidence to support the safety and efficacy of two L. curvatus probiotic candidates, PTA-16 and PTA-17 as potential probiotics for salmon.

### Example 8

### Isolation of Lactobacillus Strains

A study and project was conducted to isolate and identify bacterial strains to act as probiotics for sustainable eco-friendly antibiotic alternatives for aquaculture. Challenges and goals in aquaculture include: disease outbreaks such as with sea lice, SRS; increasing pressure to reduce antibiotic/chemical use-resistance, environmental impact; longer production cycle-2-3 years; and provision of fish meal alternatives. Probiotics have the potential to address these challenges by (1) preventing and treating diseases; modulate immune response; disease resistance; (2) accelerating growth and development; improving FCR, PER, digestibility; and (3) are eco-friendly; promote a healthy environment; and improving water quality.

Piscirickettsia salmonis causes salmon rickettsial septicemia (SRS). SRS has a significant economic impact on the aquaculture industry, in an amount $600-700 million annually in Chile alone. SRS is the number one cause of salmon mortality and morbidity, to an amount of about 70% worldwide. Another agent causing issues in aquaculture is Tenacibaculum maritimum which causes fit rot. Fit rot has a significant economic impact on the aquaculture industry, in an amount $35 million or so worldwide annually. Fit rot results in eroded mouth, skin, fins and gills and the fish are not suitable for commercial use or sale.

A study was conducted to evaluate and identity potential probiotic bacteria. Existing libraries of probiotic bacteria were evaluated and native microbes from salmon were isolated and screened. The isolated native microbes were assessed to identify and characterize them by 16S rRNA sequence (to identify species) and through whole genome sequencing. Existing libraries of probiotic bacteria were similarly evaluated. The bacteria were then characterized in vitro to assess antimicrobial activity against P. salmonis & Tenaci and through whole genome sequencing (WGS) to characterize them for digestive enzymes, bacteriocins, anti-inflammatory molecules etc. Selected strains were then evaluated for efficacy in salmon by assessing performance (FRC, weight gain) and for salmon rickettsial septicemia (SRS) prevention.

Native microbe strains were isolated from parr gut of Chilean salmon and also from grower gut of Norway salmon. Isolation from the salmon improved the likelihood that bacteria capable of growing in the cold water aquaculture conditions could be isolated. Promising Lactobacillus strains, particularly Lactilactobacillus curvatus and Lactilactobacillus sakei were isolated and identified.

A tabulation of some isolated or selected strains is shown below in TABLE 8.

TABLE 8

| | | | Antimicrobial | 15° |
|---|---|---|---|---|
| Strain | Origin | Source | Resistance | Growth |
| BVELStrain A | Chile | parr | — | 4 |
| | | gut | | |
| BSUBStrain B | Chile | parr | — | 4 |
| | | gut | | |
| LSAK204093 | Norway | grower | — | 4 |
| | | gut | | |
| LCUR204100 | Norway | grower | — | 4 |
| | | gut | | |

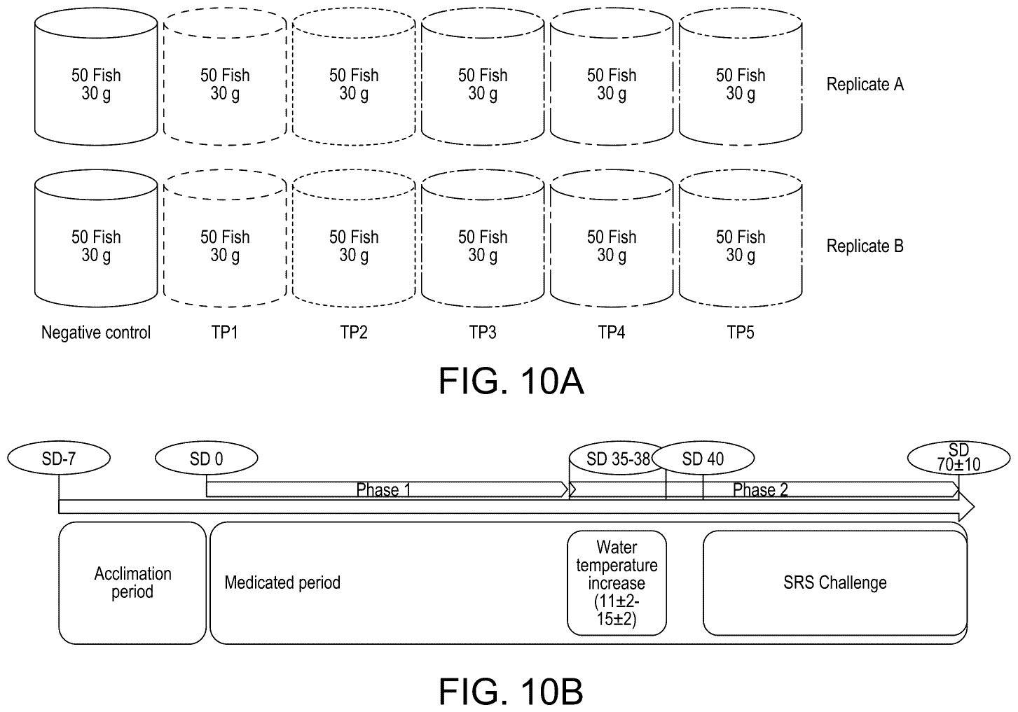

Bacillus strains and Lactobacillus strains were evaluated as probiotic candidates for performance and SRS resistance. A first study of several Bacillus strains and Salmon Lactobacillus strains 93 (ELA202093) and strain 100 (ELA204100) was conducted. Study design is as follows in TABLE 9 and aspects of the study are shown in FIG. 10. A control and 5 bacteria groups were evaluated: Negative Control is standard diet only; TP1 is B. amyloliquefaciens Strain A+B. subtilis Strain A+B. amyloliquefaciens Strain B+Standard diet; TP2 is B. velezensis Strain A+B. subtilis Strain B+B. subtilis Strain A+Standard diet; TP3 is L. curvatus ELA204100+L. sakei ELA204093+Standard diet; TP4 is L. curvatus ELA204100+L. sakei ELA204093+B. subtilis Strain A+Standard diet; TP5 is B. velezensis Strain A+B. subtilis Strain B+L. curvatus ELA204100+Standard diet.

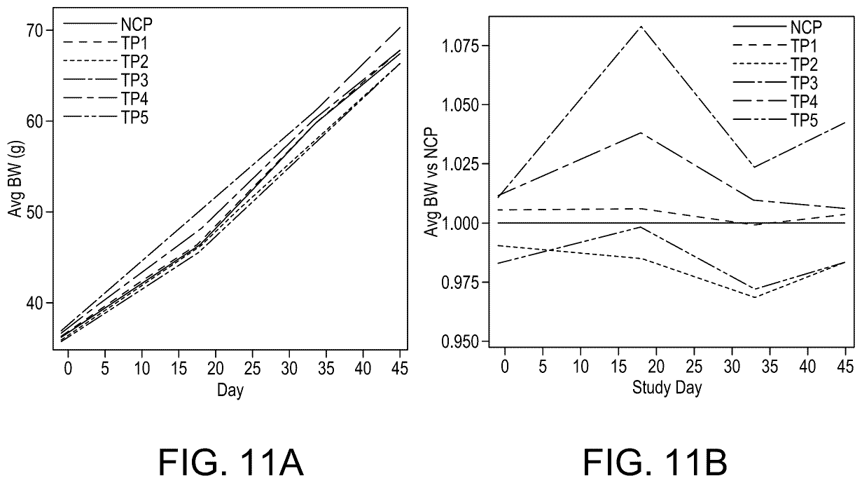

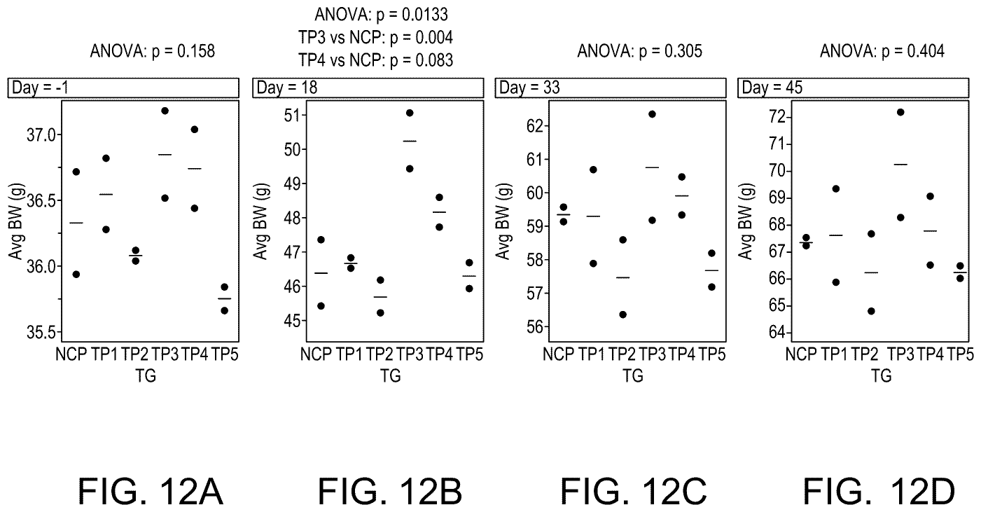

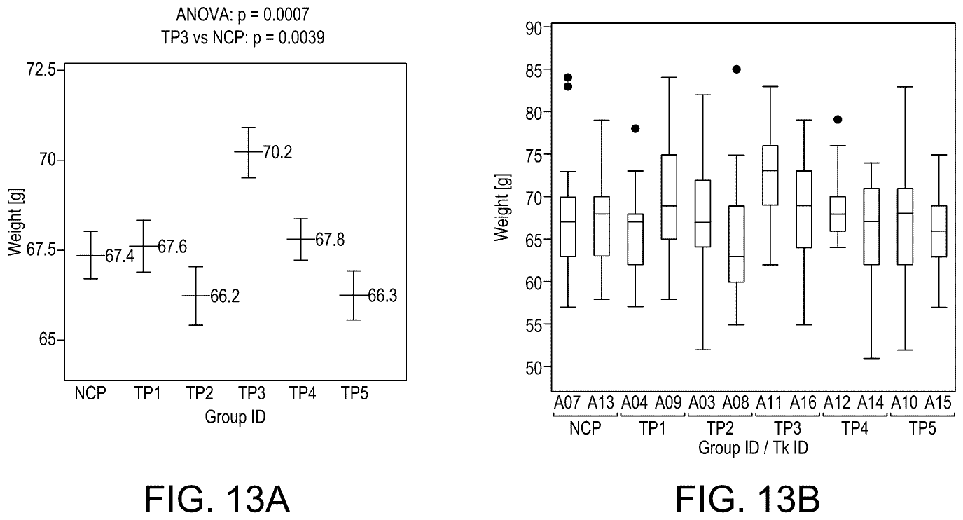

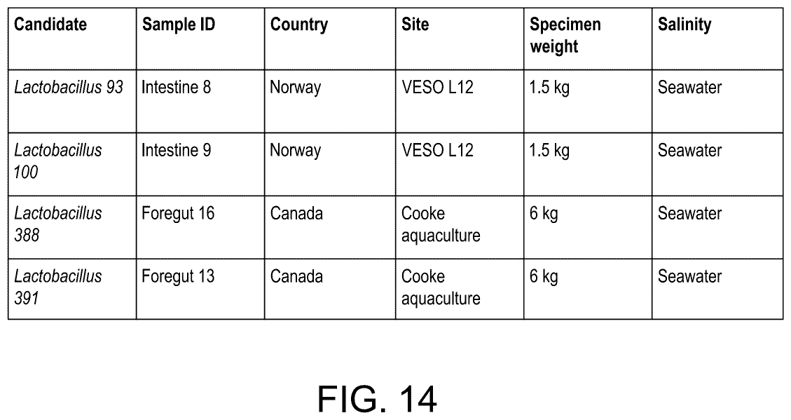

Results of the evaluation in terms of growth and body weight as a performance measure are depicted in FIGS. 11-13. Top probiotic bacteria candidate sample information is provided in FIG. 14.

TABLE 9

| Study Day | Study Activity |
|---|---|
| −7-0 | Acclimatization |
| −1 | Randomization; weight (individual) |
| All Study | Record daily observations |
| Days 0-70 | Feed issue as needed; probiotic feed will be fed all |
| | through this phase; record feed intake |
| 35 | Weight (individual); sacrifice 10 salmon per treatment - |
| | sample collection (blood and gut samples) |
| | Weight Gain Ratio (WGR); Feed Conversion Ratio (FCR); |
| | Specific Growth Rate (SGR) |
| 36-39 | Temperature conditioning for P. salmonis challenge |
| 40 | Challenge with P. salmonis |
| 50-60 | Observe for mortality/clinical signs |
| 60-70 | Sacrifice 10 salmon per treatment - sample collection |
| | (blood and gut samples) |
| | Mortality/survival (%) |
| | Microbiome profiling |
| | Gut histology |
| | Cytokines and other serum markers |

### REFERENCES

Azad M A K, Sarker M, Wan D. (2018) Immunomodulatory Effects of Probiotics on Cytokine Profiles Biomed Res Int v 2018 doi: 10.1155/2018/8063647
Cousin F J, Lynch S M, Harris H M, McCann A, Lynch D B, Neville B A, Irisawa T, Okada S, Endo A, O'Toole P W. (2015) Detection and Genomic Characterization of Motility in Lactobacillus curvatus: Confirmation of Motility in a s Species outside the Lactobacillus salivarius Clade. Appl Env Microbiol 81 (4).
Gatenby, C. (2017). From Wells to Watersheds: The Land Between Two Rivers [Web log post]. Retrieved Oct. 19, 2020, from https://usfwsnortheast.wordpress.com/2017/08/02/from-wells-to-watersheds-the-land-between-two-rivers/
Hartviksen M, Vecino J L G, Kettunen A, Myklebust R, Ruohonen K, et al. (2015) Probiotic and Pathogen Ex-vivo Exposure of Atlantic Salmon (Salmo salar L.) Intestine from Fish Fed Four Different Protein Sources. J Aquac Res Development 6:340. doi: 10.4172/2155-9546.1000340
Jones, G. 2019. Aqua Vaccines Research within REI, Elanco Meeting PP
Marine Harvest/MOWI. 2018. Salmon Farming Industry Handbook.
Marshall, S. H., Gómez, F. A., Klose, K. E. (2014) The Genus Piscirickettsia. In: Rosenberg E., DeLong E. F., Lory S., Stackebrandt E., Thompson F. (eds) The Prokaryotes. Springer, Berlin, Heidelberg
Peña A, Marshall, S. BMC Research Notes 2010, 3:101 http://www.biomedcentral.com/1756-0500/3/101
Rodriguez, J. 2015. Aquaculture: a Thriving Growth Story, Elanco Meeting PP
Rozas-Serri M, Enriquez R. 2013. Piscirickettsiosis and Piscirickettsia salmonis in fish: a review. J. Fish Dis. 37 (3)
Santos Y., F. Pazos and J. L. Barja (No. 55), Revised by Simon R. M. Jones and Lone Madsen. Tenacibaculum maritimum, causal agent of tenacibaculosis in marine fish. ICES Identification Leaflets for Diseases and Parasites of Fish and Shellfish. No. 70. 5 pp. http://doi.org/10.17895/ices.pub.4681
Yañez A, Silva H, Valenzuela K, Pontigo J, Godoy M, Troncoso J, Romero A, Figueroa J, Carcamo J, Avendaño-Herrera R. 2013. Two novel blood-free solid media for the culture of the salmonid pathogen Piscirickettsia salmonis. J. Fish Dis. 36 (587-591).

### Example 9

### Effect of Dietary Supplementation of Probiotics on Growth Performance, Immune Response, Antioxidant Properties and Survival of Atlantic Salmon (Salmo salar) in the Presence of Salmonid Rickettsial Syndrome (SRS) Challenge

Disease outbreaks cause tremendous losses to the salmonid farming industry and have become a major constraint for modern salmon farming. For microbial pathogens, there is an industry effort towards antibiotic reduction due to emergence of antibiotic-resistant bacteria and in line with best practice usage evolving across farm animal species. Probiotics are a viable, eco-friendly alternative to antibiotics. Probiotics are live microorganisms which when administered in adequate amounts confer a health benefit on the host. Originally introduced to control diseases in aquaculture, the use of probiotics has been extended to improve weight gain and performance. In aquaculture, probiotics contribute to disease prevention through competitive exclusion of pathogenic bacteria, production of antimicrobial molecules and enhancing the host immune system, which is one of the most purported benefits of using probiotics in aquaculture. The ability of probiotics to produce various digestive enzymes (better nutrient digestibility) and improve gut health (better nutrient absorption) may contribute to improved feed conversion ratio and weight gain.

Bacillus and Lactobacillus species are among the most commonly used probiotics in aquaculture. Bacillus species offer many unique advantages-being a spore former, Bacillus are known for their stability during harsh pelleting temperature, top coating, on the feed, and in the GI environment of the salmon. Bacillus are also well known for producing various antimicrobial peptides and digestive enzymes. The relative ease of fermentation/process, low cost of production and long shelf life of the final product further adds to the attractiveness of Bacillus species as probiotics for salmon.

Salmonid Rickettsial Syndrome (SRS, also known as piscirickettsiosis), caused by the facultative Gram-negative intracellular bacterium Piscirickettsia salmonis, is one of the most economically important diseases of salmon. Next to sea lice, SRS causes the most severe economic losses to Chilean salmon production, and leads to extensive antibiotic use totaling 300 tons in the seawater phase. The Chilean National Fisheries and Aquaculture Service (SERNAPESCA) estimates losses due to SRS to be over 700M USD per year, including direct losses due to disease, as well as costs related to antibiotics, vaccines and feed, and reduction in quality and size of surviving fish. The disease was first reported in Chile in 1989, and still today, 47.6% of all farmed salmonid mortalities of 2019 (including Atlantic salmon, Coho salmon and trout) are attributed to SRS. P. salmonis has also sporadically been found in other salmon-producing countries (Ireland, Norway, Canada, US and Scotland), with recent outbreaks on the west coast of Canada, but Chilean strains have higher virulence than others, as well as in other marine fish species.

In the study design, we evaluate the effect of dietary supplementation of spore-based, Bacillus probiotics on growth performance, immune response, and antioxidant properties, as well as survival of Atlantic salmon in the presence of SRS challenge. This study design is selected to minimize animal numbers while controlling for tank effect and variation due to individual fish feeding behavior. Should results indicate favorable effect of probiotics on the growth performance and survival in the presence of SRS challenge, it is anticipated that further studies would be needed to more fully explore the efficacy and benefits of probiotics. The present study evaluates the effect of dietary supplementation of Lactobacillus and Bacillus probiotics on growth performance, immune response, and antioxidant properties, as well as survival of Atlantic salmon in the presence of SRS challenge. Seven probiotic candidates will be evaluated in five combinations compared to a negative control.

This study uses a functionally blinded, replicated controlled design with facility owned, farmed Atlantic salmon held in FW. To determine the Challenge Model with target cumulative mortality of 20-30%, 120 fish are enrolled, per the defined inclusion criteria, which includes a set bodyweight range. 30 fish per tank will distributed in 4 tanks of 100 L. A disease titration with P. salmonis isolate AT17-209 is performed in single tanks at four concentrations (10-2,5, 10-3, 10-3,5 and 10-4). Fish are intraperitoneally injected with 0.1 ml of each inoculum dilution, using an appropriate needle size according to fish size as per SOP “Inoculación de patógenos en peces”. The challenge assessment is expected to finalize after 35±10 days, by comparing different specific survivals obtained by inoculum concentration.

Six hundred and twenty four (624) additional fish are selected for inclusion to the study during this process per the defined inclusion criteria, which includes a set bodyweight range. All fish are anaesthetized and individually weighed. Included fish will be distributed without intentional bias in twelve study tanks randomly allocated to 6 groups with 2 replicate tanks for each group. Study fish are offered unmedicated diet post-handling.

Post inclusion and study tank set up, study fish are acclimated for a minimum of 7 days without handling. To understand the baseline microbiome and immune parameters before treating with probiotics, 24 samples (2 samples per tank) are collected and combined from all groups on study day (SD)-1, the remaining 50 fish per tank will be anaesthetized then individually weighed. Fish bodyweights from SD-1 will be used to forecast and calculate the specific feed rate of the test diets with target minimum dose rates over the study period.

The administration of the test diets begins in the study tanks on SD 0, for a period of 35 days, from now on referred to as Phase 1 (See Table 10). To understand the effect of probiotics on the microbiome and immune parameters in the absence of SRS challenge, we collect 20 samples per treatment (10 samples per tank) before P. salmonis challenge. Upon completion of Phase 1, Phase 2 begins which considers to increase the temperature from 11±2° C. to 15±2° C., maintaining delivery of medicated feed and Challenge with P. salmonis strain AT17-209 dilution selected from challenge model. Efficacy will be evaluated based on the registration of daily mortality post-challenge and signs of disease in surviving fish. The challenge endpoint is three consecutive days without any deaths in either group in a single tank, after the onset of mortality. To understand the effect of probiotic candidates on the microbiome and immune parameters in the presence of SRS challenge, we collect 20 samples per treatment (10 samples per tank) at the end of the study. All surviving fish are euthanized with an anesthetic overdose and will be assessed for signs of disease. A total of 70±10 days of study among Phases 1 and 2.

TABLE 10
Tabular overview of treatment groups. Tank replicates
are indicated by ‘A’ and ‘B’.

| Study | Group | Replicate | | No. |
|---|---|---|---|---|
| Tank | identifi- | identifi- | | of |
| ID | cation | cation | Treatment description | fish |
| 1 | Chi- | A | B. amyloliquefaciens | 50 |
| | nook 1 | | ELA191024 + B. subtilis | |
| | (TP 1) | | ELA191105 + B. amyloliquefaciens | |
| | | | ELA202071 + Standard diet | |
| 2 | Chi- | 8 | B. amyloliquefaciens | 50 |
| | nook 1 | | ELA191024 + B. subtilis | |
| | (TP 1) | | ELA191105 + B. amyloliquefaciens | |
| | | | ELA202071 + Standard diet | |
| 3 | Chi- | A | B. velezensis ELA204005 + | 50 |
| | nook 2 | | B. subtilis ELA204016 + | |
| | (TP 2) | | B. subtilis ELA191105 + | |
| | | | Standard diet | |
| 4 | Chi- | B | B. velezensis ELA204005 + | 50 |
| | nook 2 | | B. subtilis ELA204016 + | |
| | (TP 2) | | B. subtilis ELA191105 + | |
| | | | Standard diet | |
| 5 | Chi- | A | L. curvatus ELA204100 + | 50 |
| | nook 3 | | L. sakei ELA204093 + | |
| | (TP 3) | | Standard diet | |
| 6 | Chi- | B | L. curvatus ELA204100 + | 50 |
| | nook 3 | | L. sakei ELA204093 + | |
| | (TP 3) | | Standard diet | |
| 7 | Chi- | A | L. curvatus ELA204100 + | 50 |
| | nook 4 | | L. sakei ELA204093 + | |
| | (TP 4) | | B. subtilis ELA191105 + | |
| | | | Standard diet | |
| 8 | Chi- | B | L. curvatus ELA204100 + | 50 |
| | nook 4 | | L. sakei ELA204093 + | |
| | (TP 4) | | B. subtilis ELA191105 + | |
| | | | Standard diet | |
| 9 | Chi- | A | B. velezensis ELA204005 + | 50 |
| | nook 5 | | B. subtilis ELA204016 + | |
| | (TP 5) | | L. curvatus ELA204100 + | |
| | | | Standard diet | |
| 10 | Chi- | B | B. velezensis ELA204005 + | 50 |
| | nook 5 | | B. subtilis ELA204016 + | |
| | (TP 5) | | L. curvatus ELA204100 + | |
| | | | Standard diet | |
| 11 | NCP | A | Standard diet | 50 |
| 12 | NCP | B | Standard diet | 50 |

### Schedule of the Study

A tentative outline of important study events is given in Table 11 for the milestones of the Challenge model and in Table 12 the in vivo events.

TABLE 11

| Study | |
|---|---|
| Day (±2 | |
| days) | Milestones of Challenge Model |
| Prior to | Select fish population of 120 healthy Atlantic salmon and |
| study | distribute 30 fish in each four tanks of 100 L. |
| −7 | Tank set up, start of acclimatization. Weigh all fish |
| during | Increase water temperature in tanks, from 11.0 ± 2° C. |
| acclimation | to 15 ± 2° C. |
| Daily | Maintain groups per normal husbandry, record mortalities |
| −1 | Fast |
| 0 | Challenge four dilutions. Weigh all fish |
| 35 ± 5 | End of Challenge model |

TABLE 12
Proposed schedule of in vivo study events.

| Study | |
|---|---|
| Day (±2 | Study |
| days) | Activity |
| Prior to | Health assessment |
| Study | |
| −7 | 624 fish will be enrolled and distributed in tanks (52 |
| | fish per tank). |
| | All fish will be individually measured and weighed (100% |
| | population). |
| −7 to 0 | Acclimation; Randomization |
| −1 | Weight sampling; all fish will be individually weighed |
| | (100% population) |
| | Sample collection; 2 fish per tank will be euthanized and |
| | sampled (blood and gut samples) |
| All | Record daily observations |
| Study | Sample weight each two weeks for feeding rate calculation |
| Days | Feed probiotic feed will be delivered from SD 0 and on (until |
| 0-70 | end of trial) |
| 34 | Fish will be fasted |
| 35 | Sample collection; 10 fish per treatment will be euthanized |
| | and sampled (blood and gut samples) |
| 35-38 | Temperature conditioning for P. salmonis challenge. Feed will |
| | be changed caliber gradually |
| 39 | Fish will be fasted |
| 40 | Challenge with P. salmonis. Weight sampling; all fish will be |
| | individually weighed (100% population). Fish will be fed with |
| | Nutra 60 |
| 60-70 | Weight sampling; all fish will be individually measured and |
| | weighed (100% population). Sample collection; 20 fish per |
| | treatment will be euthanized and sampled (blood and gut |
| | samples) |
| | Mortality/survival rate (%) |

### Experimental Fish and Rearing Conditions

### Experimental Animal Description

Atlantic salmon recruited to the study will be from laboratory stock maintained at the Aquarium Facility. Animal identification is summarized in Table 13.

TABLE 13
Summary of animal identification for Atlantic
salmon recruited to the study.

| | Challenge model | Probiotic evaluation |
|---|---|---|
| Species: | Atlantic salmon | Atlantic salmon |
| Fish Size: | Approximately | Approximately 40 g [30-50 g] |
| | 60-70 g average | at Day 0; No fish below 30 g will |
| | at Day 0 | be included at day 0. |
| Origin: | 2020_03 OV2AQG | 2020_03 OV2AQG |
| Age: | Parr | Parr |
| Gender: | Mixed | Mixed |
| Number: | 120 fish | 624 fish of which 600 will be |
| | | enrolled to the study. |
| Physiological | Clinically normal | Clinically normal pre-smolts |
| status: | pre- smolts | |
| Pre-treatment: | Not applicable | Not applicable |

### Animal Housing and Management

Over the study period, fish will be maintained in 100 L tanks of flow through fresh water under a photoperiod regime of 24-hour day light. Water flow during the holding period and during experimental period will be set at a rate to ensure a minimum of 2.0 total volume water exchange/hour.

If required, supplemental oxygen will be delivered to the tank water to maintain appropriate saturated oxygen levels (70-130% saturation). Water temperature for all tanks will be recorded daily. Temperature will be maintained at 12±2° C. during acclimation and Phase 1 and at 15±2° C. during challenges, with any adjustments using less than 2° C. change in a single day. Fish will not undergo any smoltification manipulation for this study.

### Inclusion to Study

Before SD-7, tank populations under consideration for the study will be screened for preinclusion to the study and a pre-study health declaration will be completed. Only tanks meeting the criteria will be anaesthetized and assessed on SD-7. If more than 600 animals are eligible for enrollment, only the first 624 eligible animals assessed will be enrolled. Participating fish must be:
Deemed by the Clinical Investigator and/or AV to be clinically healthy, sexually immature and without apparent deformities.
Average weight (see Table 13).
Data will be recorded

### Exclusion

In addition to the conditions above in inclusion, the following criteria will exclude individual fish prior to study:
Sick, weak, or stressed fish
Fish with visible signs of damage to the skin or mucous layer
Fish weighing less than 30 grams or exceeding 50 grams on day 0 phase 1

### Test Products

Five Test Products (TPs) will be assessed, each consisting of fish feed prepared with spores of Bacillus and/or with Lactobacillus as follows:
1. Chinook 1 (Test Product 1): B. amyloliquefaciens StrainA+B. subtilis StrainA+B. amyloliquefaciens StrainB+Standard diet
2. Chinook 2 (Test Product 2): B. velezensis StrainA+B. subtilis StrainB+B. subtilis StrainA+Standard diet
3. Chinook 3 (Test Product 3): L. curvatus ELA204100+L. sakei ELA204093+Standard diet
4. Chinook 4 (Test Product 4): L. curvatus ELA204100+L. sakei ELA204093+B. subtilis StrainA+Standard diet
5. Chinook 5 (Test Product 5): B. velezensis StrainA+B. subtilis StrainB+L. curvatus ELA204100+Standard diet
6. Negative Control Product: Standard diet
   The target dose range is shown calculated, per TP in Table 18 based on assayed CFU/g premix per Table 14 to Table 18.

TABLE 14
Test Product (TP) 1

| | Group identification |
|---|---|
| | TP1 |
| | Coded group name |
| | Group (G)1 |
| Description | Standard diet + B. amyloliquefaciens ELA191024 + B. |
| | subtilis ELA191105 + B. amyloliquefaciens ELA202071 |
| Probiotic strains | B. amyloliquefaciens ELA191024 + B. subtilis |
| | ELA191105 + B. amyloliquefaciens ELA202071 |
| Classification: | AAFCO/QPS, non-licensed |
| Target minimum dose | 1.63 × 10\hat{ }6 |
| rate (CFU/fish/day): | |
| Target minimum dose | 4.51 × 10\hat{ }7 |
| rate (CFU/fish BW/day): | |
| Formulation in 2436.6 g | 7.26 × 10\hat{ }10 CFUs total/1.058 g of spores/ |
| total of batch premix: | 2412 g corn starch/ 24.375 g SiO₂ |
| Assayed CFU/g of premix | 2.98 × 10\hat{ }7 |
| (total) | |
| Presentation and packaging: | White powder in 2 kg plastic canister |
| Storage: | Store in the original container. Keep the container |
| | tightly closed. Store between 4-15° C. in a dry place. |
| Lot identification: | To be included in study records |
| Expiration: | To be included in study records |

TABLE 15
Test Product (TP) 2

| | Group identification |
|---|---|
| | TP2 |
| | Coded group name |
| | Group (G)2 |
| Description | Standard diet + B. velezensis ELA204005 + B. subtilis |
| | ELA204016 + B. subtilis ELA191105 |
| Probiotic strains | B. velezensis ELA204005 + B. subtilis ELA204016 + |
| | B. subtilis ELA191105 |
| Classification: | AAFCO/QPS, non-licensed |
| Target minimum dose | 2.4 × 10\hat{ }6 CFUs/fish/day |
| rate (CFU/fish/day): | |
| Target minimum dose | 6.62 × 10\hat{ }7 |
| rate (CFU/fish BW/day): | |
| Formulation in 2434.3 g | 1.06 × 10\hat{ }11 CFUs total/3.756 g of spores/2409 g corn |
| total of batch premix: | starch/24.375 g SiO₂ |
| Assayed CFU/g of premix | 4.37 × 10\hat{ }7 |
| (total) | |
| Presentation and packaging: | White powder in 2 kg plastic canister |
| Storage: | Store in the original container. Keep the container |
| | tightly closed. Store between 4-15° C. in a dry place. |
| Lot identification: | To be included in study records |
| Expiration: | To be included in study records |

TABLE 16
Test Product (TP) 3

| | Group identification |
|---|---|
| | TP3 |
| | Coded group name |
| | Group (G)3 |
| Description | Standard diet + L. curvatus ELA204100 + L. sakei |
| | ELA204093 |
| Probiotic strains | L. curvatus ELA204100 + L. sakei ELA204093 |
| Classification: | AAFCO/QPS, non-licensed |
| Target minimum dose | 8.51 × 10\hat{ }7 |
| rate (CFU/fish/day): | |
| Target minimum dose | 2.35 × 10\hat{ }9 |
| rate (CFU/fish BW/day): | |
| Formulation in 2437.5 g | 3.78 × 10\hat{ }12 CFUs total/191.185 g lyophilized |
| total of batch premix: | Lactobacillus culture/24.375 g SiO₂ |
| Assayed CFU/g of premix | 1.55 × 10\hat{ }9 |
| (total) | |
| Presentation and packaging: | White powder in 2 kg plastic canister |
| Storage: | Store in the original container. Keep the container |
| | tightly closed. Store between 4-15° C. in a dry |
| | place. |
| Lot identification: | To be included in study records |
| Expiration: | To be included in study records |

TABLE 17
Test Product (TP) 4

| | Group identification |
|---|---|
| | TP4 |
| | Coded group name |
| | Group (G)4 |
| Description | Standard diet + L. curvatus ELA204100 + L. sakei |
| | ELA204093 + B. subtilis ELA191105 |
| Probiotic strains | L. curvatus ELA204100 + L. sakei ELA204093 + B. subtilis |
| | ELA191105 |
| Classification: | AAFCO/QPS, non-licensed |
| Target minimum dose rate | 2.47 × 10\hat{ }6 |
| (CFU/fish/day): | |
| Target minimum dose rate | 6.82 × 10\hat{ }7 |
| (CFU/fish BW/day): | |
| Formulation in 2437.3 g total | 1.01 × 10\hat{ }11 CFUs total/0.258 g of spores/191.185 g |
| of batch premix: | lyophilized Lactobacillus culture/24.375 g SiO₂ |
| Assayed CFU/g of premix | 4.5 × 10\hat{ }7 |
| (total) | |
| Presentation and packaging: | White powder in 2 kg plastic canister |
| Storage: | Store in the original container. Keep the container |
| | tightly closed. Store between 4-15° C. in a dry place. |
| Lot identification: | To be included in study records |
| Expiration: | To be included in study records |

TABLE 18
Test Product (TP) 5

| | Group identification |
|---|---|
| | TP5 |
| | Coded group name |
| | Group (G)5 |
| Description | Standard diet + B. velezensis ELA204005 + B. subtilis |
| | ELA204016 + L. curvatus ELA204100 |
| Probiotic strains | B. velezensis ELA204005 + B. subtilis ELA204016 + L. |
| | curvatus ELA204100 |
| Classification: | AAFCO/QPS, non-licensed |
| Target minimum dose rate | 2.09 × 10\hat{ }7 |
| (CFU/fish/day): | |
| Target minimum dose rate | 5.76 × 10\hat{ }8 |
| (CFU/fish BW/day): | |
| Formulation in 2434.5 g total | 9.25 × 10\hat{ }11 CFUs total/3.5 g of spores/78 g lyophilized |
| of batch premix: | Lactobacillus culture/24.375 g SiO₂ |
| Assayed CFU/g of premix | 3.8 × 10\hat{ }8 |
| (total) | |
| Presentation and packaging: | White powder in 2 kg plastic canister |
| Storage: | Store in the original container. Keep the container tightly |
| | closed. Store between 4-15° C. in a dry place. |
| Lot identification: | To be included in study records |
| Expiration: | To be included in study records |

TABLE 19

| | Group identification |
|---|---|
| | NCP |
| | Coded group name |
| | Group (G)1 |
| Description | Standard diet |
| Investigational | NA |
| Probiotic Product | |
| (IPP) name: | |
| IPP classification: | NA |
| IPP dose rate: | NA |
| IPP formulation: | NA |
| IPP presentation: | NA |
| IPP storage: | NA |
| IPP lot identification: | NA |
| IPP expiration: | NA |

### Treatment Preparation and Validation

Test diets will be formulated with probiotics by top coating the premix to the feed at an incorporation rate of approximately 750.0 g premix/10 kg feed. This inclusion rate considers the calculations and parameters described in Table 18, including an adjustment of SFR to 110% of expected rate during the treatment period to allow all fish opportunity to feed. Bodyweight is estimated to be ≈35-45 g on SD-1. Medicated (probiotic) diet preparation will be documented.

TABLE 20
Calculations for projected weekly SFR and dose rates over the study period. Minimum daily dose rates (in CFU/fish Bw/day)
are projected in Study Weeks 4 and 5. Dose rate calculations consider the assayed CFU/g premix per Table 14 to Table 18.

| | | | Phase 1 | | | | | Phase 2 | | | | |
|---|---|---|---|---|---|---|---|---|---|---|---|---|
| | | | Medicated period + Water T° increase (Week 6) + SRS Challenge | | | | | | | | | |
| | | Week | Week | Week | Week | Week | Week | Week | Week | Week | Week | Week |
| | | 0 | 1 | 2 | 3 | 4 | 5 | 6 | 7 | 8 | 9 | 10 |
| % Specific | | 100% | 100% | 100% | 100% | 100% | 100% | 100% | 100% | 100% | 100% | 100% |
| growth | | | | | | | | | | | | |
| rate (SGR) | | | | | | | | | | | | |
| Initial | | 30.0 | 34.7 | 40.0 | 45.9 | 52.52 | 59.8 | 68.0 | 79.04 | 92.8 | 108.0 | 125.3 |
| weight (g) | | | | | | | | | | | | |
| Average | | 32.3 | 37.3 | 42.9 | 49.2 | 56.1 | 63.9 | 73.5 | 85.9 | 100.4 | 116.6 | 135.0 |
| weight (gr) | | | | | | | | | | | | |
| Final | | 34.7 | 40.0 | 45.9 | 52.5 | 59.77 | 68.0 | 79.0 | 92.8 | 108.01 | 125.3 | 144.7 |
| weight (g) | | | | | | | | | | | | |
| Temp. (° C.) | | 11.0 | 11.0 | 11.0 | 11.0 | 11.0 | 11.0 | 13.0 | 15.0 | 15.0 | 15.0 | 15.0 |
| Specific feed | | 2.06 | 2.06 | 1.93 | 1.93 | 1.84 | 1.84 | 2.14 | 2.29 | 2.16 | 2.11 | 2.05 |
| rate (SFR; | | | | | | | | | | | | |
| % Bw/day) | | | | | | | | | | | | |
| 110% SFR | | 2.27 | 2.27 | 2.12 | 2.12 | 2.02 | 2.02 | 2.35 | 2.52 | 2.38 | 2.32 | 2.26 |
| Fish | | 1.615 | 1.865 | 2.145 | 2.46 | 2.805 | 3.195 | 3.675 | 4.295 | 5.02 | 5.83 | 6.75 |
| biomass/tank | | | | | | | | | | | | |
| (kg) | | | | | | | | | | | | |
| Feed/tank/day | | 0.037 | 0.042 | 0.045 | 0.052 | 0.057 | 0.065 | 0.086 | 0.108 | 0.119 | 0.135 | 0.153 |
| (kg) | | | | | | | | | | | | |
| Feed/fish/day | | 0.732 | 0.847 | 0.909 | 1.043 | 1.133 | 1.291 | 1.727 | 2.165 | 2.390 | 2.705 | 3.051 |
| (g) | | | | | | | | | | | | |
| Projected | TP1 | NA | 1.89E+06 | 2.03E+06 | 2.33E+06 | 2.53E+06 | 2.88E+06 | 3.86E+06 | 4.84E+06 | 5.34E+06 | 6.04E+06 | 6.82E+06 |
| dose | TP2 | | 2.77E+06 | 2.98E+06 | 3.42E+06 | 3.71E+06 | 4.23E+06 | 5.66E+06 | 7.09E+06 | 7.83E+06 | 8.86E+06 | 9.99E+06 |
| (CFU/ | TP3 | | 9.84E+07 | 1.06E+08 | 1.21E+08 | 1.32E+08 | 1.50E+08 | 2.01E+08 | 2.52E+08 | 2.78E+08 | 3.14E+08 | 3.55E+08 |
| fish/day) | TP4 | | 2.86E+06 | 3.07E+06 | 3.52E+06 | 3.82E+06 | 4.36E+06 | 5.83E+06 | 7.31E+06 | 8.06E+06 | 9.13E+06 | 1.03E+07 |
| | TP5 | | 2.41E+07 | 2.59E+07 | 2.97E+07 | 3.23E+07 | 3.68E+07 | 4.92E+07 | 6.17E+07 | 6.81E+07 | 7.71E+07 | 8.70E+07 |
| Projected | TP 1 | NA | 5.07E+07 | 4.74E+07 | 4.74E+07 | 4.51E+07 | 4.51E+07 | 5.25E+07 | 5.63E+07 | 5.32E+07 | 5.18E+07 | 5.05E+07 |
| dose | TP 2 | | 7.43E+07 | 6.94E+07 | 6.94E+07 | 6.62E+07 | 6.62E+07 | 7.70E+07 | 8.25E+07 | 7.79E+07 | 7.60E+07 | 7.40E+07 |
| (CFU/ | TP 3 | | 2.64E+09 | 2.46E+09 | 2.46E+09 | 2.35E+09 | 2.35E+09 | 2.73E+09 | 2.93E+09 | 2.77E+09 | 2.70E+09 | 2.63E+09 |
| fish | TP 4 | | 7.66E+07 | 7.16E+07 | 7.16E+07 | 6.82E+07 | 6.82E+07 | 7.93E+07 | 8.51E+07 | 8.03E+07 | 7.83E+07 | 7.63E+07 |
| Bw/day; | TP 5 | | 6.47E+08 | 6.04E+08 | 6.04E+08 | 5.76E+08 | 5.76E+08 | 6.70E+08 | 7.18E+08 | 6.78E+08 | 6.61E+08 | 6.44E+08 |
| CFU/kg) | | | | | | | | | | | | |

A pilot scale mixer will be used for preparation of the 5 test diets (TP1, TP2, TP3, TP4 and TP5) in a 10 kg batch size as per the recipe outlined in Table 21. Medicated feed will be prepared with two feed pellet sizes: Nutra Supreme HE 30 and Nutra Supreme HE 60, which will be delivered according to fish size (Nutra Supreme HE 30 will be delivered when fish are weighing up to approximately 60 g average Bw, and Nutra Supreme HE 60, when fish are over 60 g), and will be prepared in equal way. Medicated feed will be prepared using the recipe identified in 3 phases as follows:
1—Feed and premix mix for 30 seconds (dry mixing)
2—Fish oil addition over 30 seconds, no more than 0.5% oil
3—Keep mixing for another 60 seconds (wet mixing)

TABLE 21
Test diet recipes, per 10 kg batch size

| | Diet | | | Oil | Batch |
|---|---|---|---|---|---|
| Group | name | Feed (g) | Probiotic premix (g) | (g) | total (g) |
| TP1 | Diet 1 | 9,150 | 750 g (2.23 × 10\hat{ }10 | 100 | 10,000 |
| | | | CFUs total) | | |
| TP2 | Diet 2 | 9,150 | 750 g (3.28 × 10\hat{ }10 | 100 | 10,000 |
| | | | CFUs total) | | |
| TP3 | Diet 3 | 9,150 | 750 g (1.16 × 10\hat{ }12 | 100 | 10,000 |
| | | | CFUs total) | | |
| TP4 | Diet 4 | 9,150 | 750 g (3.38 × 10\hat{ }10 | 100 | 10,000 |
| | | | CFUs total) | | |
| TP5 | Diet 5 | 9,150 | 750 g (2.85 × 10\hat{ }11 | 100 | 10,000 |
| | | | CFU total) | | |
| NCP | Diet 6 | 9,900 | — | 100 | 10,000 |

One hundred grams duplicated feed samples are collected from each batch of medicated feed and recorded on CRF ‘Medicated feed preparation’. Samples are collected by the Clinical Investigator and stored frozen at −20° C. in airtight-labeled plastic bags for future analysis.

### Treatment Administration

During acclimation fish will be fed a commercially available and complete unmedicated commercial salmonid diet of the same pellet size and approximate composition as the negative control diet, provided by Skretting.

During medication periods phase 1 & 2, Fish will be fed approximately 110% of the specific feed rate (SFR) identified using a Skretting feed table (projected per Table 20). Exploratory weight sampling will be performed approximately every 14 days and will be registered on the CRF ‘Weight sampling’. Average bodyweights will allow adjusting feed amounts to be delivered to each tank.

Medicated feed quantity will be calculated for 14 days and thereafter be pre-weighed for assuming a constant daily SFR over the administration period, and stored in labelled plastic bags with date, group and tank identification.

Exploratory weight sampling will be performed approximately every 14 days. Average Body weights will allow adjusting feed amounts to be delivered to each tank and every 7 days feed will be calculated based on projected body weight.

### Body Weight and Length Assessments

Body weight data will be recorded from individual anaesthetized fish on tank set up, SD-1, SD40 and at the end of study, the survivors. Weigh scales will be calibrated on each day prior to use. The length (nose to fork length) will be recorded in all sampled fish and survivors.

### Sample Preparation for Microbiome and Metabolomics

### Profiling

For the gut samples destined to microbiome and metabolomic profiling, an incision to the ventral surface of the fish will be made to expose and then incise the gut. Any feces remaining within the hindgut will be manually removed using paper towel. Whole gut samples will be collected using disposable or sterilized forceps and placed in sterile whirlPak bags, then stored at −20 C. To minimize cross-contamination, processing of samples will use disposable equipment whenever possible and new or cleaned equipment will be used for each fish. Non-disposable items will be cleaned by washing with a detergent then a strong organic solvent (e.g. ethanol, acetone) between samples. Fresh disposable gloves and disposable scalpels will be used for each fish and the necropsy surface cleaned between animals. Each fish will be processed and samples collected individually with care to maintain CID to the various samples as they are taken.

### Assessment of Efficacy

During the challenge period of the study, any moribund fish that reach a humane endpoint (as described below) will be removed from a tank, euthanized, and counted as a mortality. A fish is selected for humane endpoint if any of the two following criteria is observed:
Criterion 1: Fish is in lateral-recumbency, dorsolateral-recumbency, or dorsalrecumbency on the bottom of the tank or floating at the water surface.
Criterion 2: Fish is unable to achieve or maintain a normal orientation for a salmonid in the water column.

Any mortality or terminally moribund fish noticed during the challenge observation period will be removed from the tank; will be recorded on CRF ‘Necropsy’ and assigned an increasing and sequentially numbered case identification number (CID), beginning with ‘1’. Mortalities and moribund fish will be noted as ‘M’, while survivors will be noted as ‘S’. An evaluation of clinical signs of SRS will be documented for all challenged fish (mortalities and survivors).

Clinical signs of SRS include one or more of the following observations: abdominal swelling, pale gills, petechial and ecchymotic haemorrhages on the fin bases, skin ulceration, subcapsular grey/yellow mottled liver coloration, small ring shaped foci of the liver, and yellowish mucous filled intestines (Rozas and Enríquez, 2014).

The SRS Indirect Immunofluorescence Antibody Test (SRS-IFAT) will be used for mortality confirmation in the efficacy assessment. Samples of the posterior kidney will be collected by inserting a first use loop into the tissue to prepare a smear on a glass slide. Samples for IFAT will be delivered and further processed by the Diagnostic Laboratory (at Puerto Varas Study Site) as soon as possible. Samples that cannot be processed on the same day will be stored at 6±4° C. and processed as soon as possible. All samples delivered to the Diagnostic Laboratory will be recorded on CRF ‘Derivation of sample’. For a mortality to be considered specific for SRS, at least one of the above clinical signs should be observed and/or the posterior kidney should be positive in the SRS-IFAT.

### Definition of Efficacy

TP efficacy will be defined as meeting one or more of the following criteria:

1. Relative improvement of bodyweight and growth parameters including but not limited to Weight Gain Ratio (WGR) and Specific Growth Rate (SGR), of the TP in comparison to the NCP. Calculations may consider the following periods: Phase I, Phase II and the combination of Phase I+II.

2. Improvement in survival of the TP in comparison to the NCP. Calculations may consider the following periods: Phase I, Phase II and the combination of Phase I+II.

3. A negative association of Absolute Risk Reduction (ARR) between the TP and fish mortality/signs of disease. Calculations may consider the following periods: Phase I, Phase II and the combination of Phase I+II. Mortalities and moribund fish will be used to calculate the ARR

### Piscirickettsia Challenge Assessment

### Preparation

Briefly, isolate AT17-209 will be thawed from frozen stocks of challenge seed and cultured in the Chinook salmon embryo cell line CHSE-214 with either minimum essential medium (MEM) or L-15 maintenance medium, with the addition of 10% fetal bovine serum (FBS) and without antibiotics, until a cytopathic effect (CPE) of 80-90% is obtained, according to SOP ‘Infección de células con un patógeno intracelular’. For the Challenge of Phase 2 in the efficacy assessment, the challenge material to be used will be defined based on the challenge model, targeting a minimum of 20-40% of SRS-specific mortality.

### Administration and Dosing

No more than seven days prior to beginning a challenge, a bulk average weight of the fish will be determined. Correct needle size for challenge injections will be determined by euthanizing a few fish and checking needle length required to deliver an i.p. injection. Groups of fish will be netted into an anesthetic bath, and once anesthetized, removed from the bath and i.p. injected one pelvic fin length anterior to the pelvic girdle on the ventral mid-line with 0.1 ml of challenge material and finally returned to their holding tank. Challenge administration details will be recorded.

### Description of Statistical Methods and Calculations

The tank will be the experimental unit and fish will be the observational unit. Variable calculations and statistical analyses will be performed for individual phases and the overall study. Growth performance variables may include but are not limited to Weight Gain Ratio (WGR) and Specific Growth Rate (SGR). The effect of treatment on growth performance variables will be analyzed using a one-way analysis of variance. All pairwise comparisons will be evaluated using a two-tail student's t-test. Differences in survival between treatments will be evaluated using Kaplan-Meier analysis. Mortality may also be evaluated using a mixed model procedure, assuming a binomial distribution and logit link. Analyses will be performed using JMP version 14.0 or higher (SAS Institute, Inc. Cary NC).

### Example 10

### Effect of Dietary Supplementation of Lactobacillus Probiotics on Growth Performance, Immune Response, Antioxidant Properties of Atlantic Salmon

Disease outbreaks cause tremendous losses to the salmonid farming industry and have become a major constraint for modern salmon farming. For microbial pathogens, there is an industry effort towards antibiotic reduction due to emergence of antibiotic-resistant bacteria and in line with best practice usage evolving across farm animal species. Probiotics are a viable, eco-friendly alternative to antibiotics. Probiotics are live microorganisms which when administered in adequate amounts confer a health benefit on the host. Originally introduced to control diseases in aquaculture, the use of probiotics has been extended to improve weight gain and performance. In aquaculture, probiotics contribute to disease prevention through competitive exclusion of pathogenic bacteria, production of antimicrobial molecules and enhancing the host immune system, which is one of the most purported benefits of using probiotics in aquaculture. The ability of probiotics to produce various digestive enzymes (better nutrient digestibility) and improve gut health (better nutrient absorption) may contribute to improved feed conversion ratio and weight gain.

In the proposed study design, we evaluate the effect of dietary supplementation of Lactobacillus probiotics on growth performance, immune response, and antioxidant properties.

This pilot study design is selected to minimize animal numbers while controlling for tank effect and variation due to individual fish feeding behavior.

Four probiotic candidates (four strains of Lactobacillus) are evaluated in two combinations (TP1 and TP2) compared to a negative control, to determinate their effect on growth performance, immune response, and antioxidant properties.

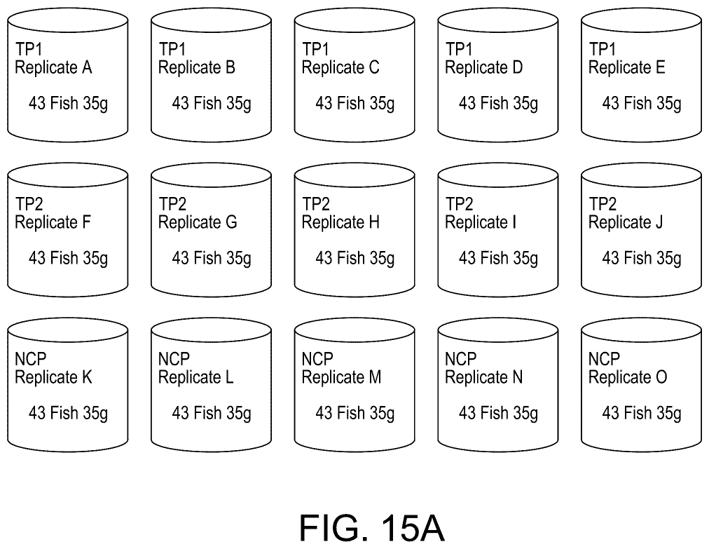

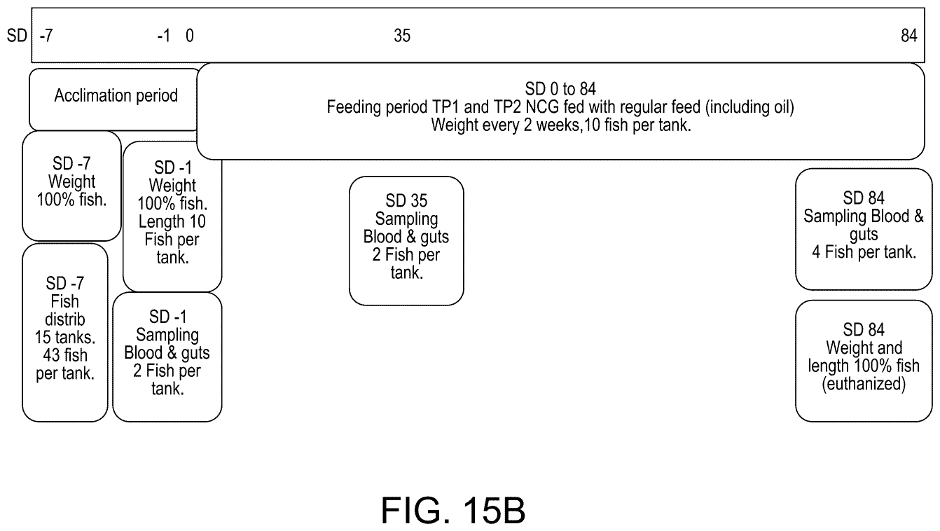

Five hundred and eighty-five (585), plus 60 extra fish (at 10%, totaling 645) will be selected for inclusion to the study during this process per the defined inclusion criteria, which includes a set bodyweight range. On day-7, all fish will be anaesthetized and individually weighed. Included fish will be distributed without intentional bias in fifteen study tanks (43 fish per tank) randomly allocated to 3 groups with 5 replicate tanks for each group (see table 22 and FIG. 15A). Study fish will be offered unmedicated diet post-handling. One hundred and fifty fish (50 per group and 10 per tank) will be lengthened.

Post inclusion and study tank set up; study fish will be acclimated for a minimum of 5 days without handling. To understand the baseline microbiome and immune parameters before treating with probiotics, 10 samples (2 samples per tank) per group will be collected and combined from all groups on study day (SD)-1. The remaining 41 fish per tank will be anaesthetized then individually weighed. Fish bodyweights from SD-1 will be used to forecast and calculate the specific feed rate of the test diets with target minimum dose rates over the study period.

The administration of the test diets will begin in the study tanks on SD 0, for a period of 84 days, or until fish weight average raise up to 125 grams, as established as a limit of biomass feasible to keep under proper conditions for the 100 L tanks. To avoid a high density at the end period of the study, the number of fish per tank can be decreased on the discretion of the Investigator, based on the following parameters: an increase of more than 48 Kg/m³ per tank and/or; fish reach an average weight higher than 125 grams. The objective of this is to ensure proper continuity of the study by completing the period of 10 weeks (as minimum) and to reach the goal of 12 weeks. In case that number are decreased, equal number per tank will be applied. A minimum number of 32 fish per tank should be considered to respect to reach at the end of the study, in order to keep statistical validation (the minimum difference may vary to 7 grams between different groups). Depopulated fish will be euthanized to be weighed and measured its length.

To understand the effect of probiotics on the microbiome and immune parameters, 10 samples per treatment (2 samples per tank) will be collected at day 35 and 20 samples per treatment (4 samples per tank) will be collected at the end of the study period (SD84). In addition, at SD84 all fish will be euthanized with an anesthetic overdose and will be weighted and lengthened (FIG. 15B).

TABLE 22
Tabular overview of treatment groups. Tank replicates are indicated
by letters ‘A to E’, ‘F to J’ and ‘K to O’.

| Study | Group | Replicate | | No. of |
|---|---|---|---|---|
| Tank ID | identification | identification | Treatment description | fish |
| 1 | TP 1 | A | Standard diet + L. curvatus ELA204100 + | 43 |
| | | | L. curvatus ELA204093. | |
| 2 | TP 1 | B | Standard diet + L. curvatus ELA204100 | 43 |
| | | | L. curvatus ELA204093. | |
| 3 | TP 1 | C | Standard diet + L. curvatus | 43 |
| | | | ELA204100 + L. curvatus | |
| | | | ELA204093. | |
| 4 | TP 1 | D | Standard diet + L. curvatus ELA204100 + | 43 |
| | | | L. curvatus ELA204093 | |
| 5 | TP 1 | E | Standard diet + L. curvatus ELA204100 + | 43 |
| | | | L. curvatus ELA204093. | |
| 6 | TP 2 | F | Standard diet + L. curvatus ELA214388 + | 43 |
| | | | L. sakei ELA214391 | |
| 7 | TP 2 | G | Standard diet + L. curvatus ELA214388 + L. | 43 |
| | | | sakei ELA214391 | |
| 8 | TP 2 | H | Standard diet + L. curvatus ELA214388 + L. | 43 |
| | | | sakei ELA214391 | |
| 9 | TP 2 | I | Standard diet + L. curvatus ELA214388 + L. | 43 |
| | | | sakei ELA214391 | |
| 10 | TP 2 | J | Standard diet + L. curvatus ELA214388 + L. | 43 |
| | | | sakei ELA214391 | |
| 11 | NCP | K | Standard diet | 43 |
| 12 | NCP | L | Standard diet | 43 |
| 13 | NCP | M | Standard diet | 43 |
| 14 | NCP | N | Standard diet | 43 |
| 1 | N | O | Standard diet | 43 |

TP—Test Product;
NCP—Negative Control Product

Schedule of the study—A tentative outline of important study events is given in Table 23 for the milestones of the study including the in vivo events; calendar dates and weeks are subject to change without study amendment and will be documented in the study master file (SMF).

TABLE 23
Proposed schedule of in vivo study events. Calendar dates
will vary with fish growth and will be reported in the FSR.

| Study Day | |
|---|---|
| (±2 days) | Study Activity |
| Prior to | Health assessment, fish pre-inclusion. |
| Study | |
| −7 | 645 fish will be enrolled and distributed in fifteen 100 L tanks |
| | (43 fish per tank). All fish will be individually weighed (100% |
| | population) and 10 fish per tank will be measured for length. |
| −7 to 0 | Acclimation; Tank randomization. |
| | Weight sampling: all fish will be individually weighed |
| | (100% population). |
| −1 | Sample collection: 2 fish per tank will be euthanized and |
| | sampled (blood and gut samples). |
| All Study | Record daily observations. |
| Days 0-84 | Sample weight each two weeks for feeding rate calculation |
| | (10 fish per tank). |
| | Study diets will be delivered from SD 0 until end of trial. |
| 35 | Sample collection: 10 fish per treatment will be euthanized, |
| | weighted and sampled for blood and gut samples. |
| 84 | All fish will be individually measured and weighed (100% |
| | population, euthanized). |
| | Sample collection: 20 fish per treatment (4 per tank) |
| | will be anesthetized and euthanized for blood and gut |
| | samples. |

Day 0 is defined as the beginning of Test products (TPs).

### Experimental Fish and Rearing Conditions

### Experimental Animal Description

Atlantic salmon recruited to the study will be from laboratory stock maintained at the Aquarium Facility. Animal identification is summarized in Table 23. Disease and treatment history will be recorded.

TABLE 23
Summary of animal identification for Atlantic
salmon recruited to the study.

| | Probiotic performance evaluation |
|---|---|
| Species: | Atlantic salmon |
| Fish Size: | Approximately 35 g [30-40 g] at Day 0; No fish below |
| | 30 g will be included at day 0. |
| Origin: | 2020_10_OV4AQG |
| Age: | Parr |
| Gender: | Mixed |
| Number: | 645 fish to be enrolled to the study. |
| Physiological | Clinically normal pre-smolts. |
| status: | |
| Pre-treatment: | Not applicable. |

### Animal Housing and Management

Over the study period, fish will be maintained in 100 L tanks of flow through fresh water under a photoperiod regime of 24-hour day light. Water flow during the holding period and during experimental period will be set at a rate to ensure a minimum of 2.0 total volume water exchange/hour. If required, supplemental oxygen will be delivered to the tank water to maintain appropriate saturated oxygen levels (70-130% saturation). Water temperature for all tanks will be recorded daily. Temperature will be maintained at 11.0±1.0° C. during the whole study.

### Animal Selection and Identification

A single pre-study assessment will occur on approximately SD-7. No individual tagging or identification will be undertaken in this study.

### Inclusion to Study

Before SD-7, tank populations under consideration for the study will be screened for pre-inclusion to the study and a pre-study health declaration will be completed using the CRF ‘Fish health declaration’. If more than 645 animals are eligible for enrollment, only the first 645 eligible animals assessed will be enrolled. Only fish meeting the criteria will be anaesthetized and assessed on SD-7. Participating fish must be:
Deemed by the Clinical Investigator and/or AV to be clinically healthy, sexually immature and without apparent deformities.
Average weight 33-37 g.
Data will be recorded

### Exclusion

In addition to the conditions above in inclusion, the following criteria will exclude individual fish prior to study:
Sick, weak, or stressed fish
Fish with visible signs of damage to the skin or mucous layer #Fish weighing less than 30 grams or exceeding 42 grams on SD 0.

### Post-Inclusion Removal (Withdrawal)

For any tank classification if either study treatment or observations are discontinued, the reason will be reported directly to the Investigator and noted using the CRF ‘Note to file’. The reasons could include:
Concomitant disorders that may interfere with the evaluation of response to treatment; at the discretion of the Clinical Investigator in consultation with the Sponsor's Representative (SR) and/or Study Advisors (SA).
The need to remove live animals affected by an adverse event (AE) will be made on an individual case basis by the Clinical Investigator in consultation with the SR/SAs.
Protocol deviation(s) that compromise integrity of the study.

### Acclimization and Study Tank Set Up

After inclusion to the study, fish will be allocated to study tanks as part of the handling inclusion process without intentional bias and acclimated per normal site husbandry.

Post study tank set up; fish will be acclimated for at least seven days before they are re-handled. Otherwise, animals will be maintained per normal husbandry procedures.

### Test Products

Two Test Products (TPs) will be assessed, each consisting of fish feed prepared with spores of Lactobacillus as follows:
1. Chinook 1 (Test Product 1): Standard diet+L. curvatus ELA204100+L. curvatus ELA204093.
2. Chinook 2 (Test Product 2): Standard diet+L. curvatus ELA214388+L. sakei ELA214391
3. Negative Control Product: Standard diet

The target dose range is shown calculated, per TP in Table 20. Calculations for projected weekly SFR and dose rates over the study period. Minimum daily dose rates (in CFU/fish Bw/day) are projected in Study Weeks 4 and 5. Dose rate calculations consider the assayed CFU/g premix per Table 24 and 25. DoseRE PROJMinim based on assayed CFU/g premix per tables 24 and 25.

TABLE 24
Test Product 1 (TP1)

| | Group identification |
|---|---|
| | TP1 |
| | Coded group name |
| | Group (G)1 |
| Description | Standard diet + L. curvatus ELA204100 + L. |
| | curvatus ELA204093 |
| Probiotic strains | L. curvatus ELA204100 + L. curvatus ELA204093 |
| Classification: | AAFCO/QPS, non-licensed |
| Target minimum dose rate | 8.51 × 10\hat{ }7 |
| (CFU/fish/day): | |
| Target minimum dose rate | 2.35 × 10\hat{ }9 |
| (CFU/fish BW/day): | |
| Formulation in 3527 g total | 4.44 × 10\hat{ }12 CFUs total/578 g lyophilized Lactobacillus |
| of batch premix: | culture/24.375 g SiO₂ |
| Assayed CFU/g of premix | 1.26 × 10\hat{ }9 |
| (total) | |
| Presentation and packaging: | White powder in 2 kg plastic canister |
| Storage: | Store in the original container. Keep the container tightly |
| | closed. Store between 4-15° C. in a dry place. |
| Lot identification: | To be included in study records |
| Expiration: | To be included in study records |

TABLE 25
Test Product 1 (TP2)

| | Group identification |
|---|---|
| | TP2 |
| | Coded group name |
| | Group (G)2 |
| Description | Standard diet + L. curvatus ELA214388 + L. |
| | sakei ELA214391 |
| Probiotic strains | L. curvatus ELA214388 + L. sakei ELA214391 |
| Classification: | AAFCO/QPS, non-licensed |
| Target minimum dose rate | 8.51 × 10\hat{ }7 |
| (CFU/fish/day): | |
| Target minimum dose rate | 2.35 × 10\hat{ }9 |
| (CFU/fish BW/day): | |
| Formulation in 3527 g total | 4.31 × 10\hat{ }12 CFUs total/831 g lyophilized Lactobacillus |
| of batch premix: | culture/24.375 g SiO₂ |
| Assayed CFU/g of premix | 1.21 × 10\hat{ }9 |
| (total) | |
| Presentation and packaging: | White powder in 2 kg plastic canister |
| Storage: | Store in the original container. Keep the container tightly |
| | closed. Store between 4-15° C. in a dry place. |
| Lot identification: | To be included in study records |
| Expiration: | To be included in study records |

TABLE 26
Negative Control Product

| | Group identification |
|---|---|
| | NCP |
| | Coded group name |
| | Group (G)1 |
| Description | Standard |
| | diet |
| Investigational | |
| Probiotic Product | NA |
| (IPP) name: | |
| IPP classification: | NA |
| IPP dose rate: | NA |
| IPP formulation: | NA |
| IPP presentation: | NA |
| IPP storage: | NA |
| IPP lot identification: | NA |
| IPP expiration: | NA |

### Treatment Preparation and Validation

Test diets will be formulated with probiotics by top coating the premix to the feed at an incorporation rate of approximately 500.0 g premix/10 kg feed. This inclusion rate considers the calculations and parameters described in Table 27 including an adjustment of SFR to 110% of expected rate during the treatment period to allow all fish opportunity to feed. Bodyweight is estimated to be ≈30-40 g on SD-1. Medicated (probiotic) diet preparation will be documented. Feed preparation as NCP or TP diets (‘Study Diets’): The same manufacturer provided, complete pelleted diet for salmon will be used as a base to prepare the NCP and the two TP diets. The respective probiotic candidates will be mixed with fish oil and sprayed onto the feed and dried at room temperature and stored at 4° C. The NCP will likewise be mixed with fish oil sprayed into the feed (but no probiotic), dried at room temperature, and stored per the TP. The NCP diet will be prepared first, prior to the TP diets.

TABLE 27
Calculations for projected weekly SFR and dose rates over the study period.
Minimum daily dose rates (in CFU/fish Bw/day) are projected in Study Weeks
4 and 5. Dose rate calculations consider the assayed CFU/g premix.

| | | Medicated Period | | | | | | |
|---|---|---|---|---|---|---|---|---|
| | | Week −1 | Week 1 | Week 2 | Week 3 | Week 4 | Week 5 | Week 6 |
| % Specific | | 100% | 100% | 100% | 100% | 100% | 100% | 100% |
| growth rate | | | | | | | | |
| (SGR) | | | | | | | | |
| Initial | | 31.0 | 35.8 | 41.4 | 47.4 | 54.3 | 61.8 | 69.9 |
| weight (g) | | | | | | | | |
| Average | | 33.4 | 38.6 | 44.4 | 50.8 | 58.0 | 65.8 | 74.6 |
| weight (gr) | | | | | | | | |
| Final | | 35.8 | 41.4 | 47.4 | 54.3 | 61.8 | 69.9 | 79.2 |
| weight (g) | | | | | | | | |
| Temp. (° C.) | | 10.8 | 10.8 | 10.8 | 10.8 | 10.8 | 10.8 | 10.8 |
| Specific feed | | 1.90 | 1.90 | 1.80 | 1.80 | 1.70 | 1.70 | 1.60 |
| rate (SFR; % | | | | | | | | |
| Bw/day) | | | | | | | | |
| 110% SFR | | 2.05 | 2.05 | 1.92 | 1.92 | 1.83 | 1.76 | 1.76 |
| Fish | | 1426 | 1549 | 1758 | 1987 | 2239 | 2516 | 2820 |
| biomass/ | | | | | | | | |
| tank (g) | | | | | | | | |
| Feed/tank/ | | 27 | 29 | 32 | 36 | 38 | 43 | 45 |
| day | | | | | | | | |
| (kg) | | | | | | | | |
| Feed/fish/day | | 0.6 | 0.7 | 0.8 | 0.9 | 0.9 | 1.0 | 1.2 |
| (g) | | | | | | | | |
| Projected | TP1 | 3.78E+07 | 4.41E+07 | 5.04E+07 | 5.67E+07 | 5.67E+07 | 6.30E+07 | 7.56E+07 |
| dose | TP2 | 3.63E+07 | 4.24E+07 | 4.84E+07 | 5.45E+07 | 5.45E+07 | 6.05E+07 | 7.26E+07 |
| (CFU/ | | | | | | | | |
| fish/day) | | | | | | | | |
| Projected | TP 1 | 1.13E+09 | 1.14E+09 | 1.14E+09 | 1.12E+09 | 9.78E+08 | 9.57E+08 | 1.01E+09 |
| dose | TP 2 | 1.09E+09 | 1.10E+09 | 1.09E+09 | 1.07E+09 | 9.39E+08 | 9.19E+08 | 9.73E+08 |
| (CFU/ | | | | | | | | |
| fish | | | | | | | | |
| Bw/day; | | | | | | | | |
| CFU/kg) | | | | | | | | |

| | | Medicated Period | | | | | |
|---|---|---|---|---|---|---|---|
| | | Week 7 | Week 8 | Week 9 | Week 10 | Week 11 | Week 12 |
| % Specific | | 100% | 100% | 100% | 100% | 100% | 100% |
| growth rate | | | | | | | |
| (SGR) | | | | | | | |
| Initial | | 79.2 | 89.3 | 100.4 | 112.2 | 125.4 | 139.8 |
| weight (g) | | | | | | | |
| Average | | 84.2 | 94.8 | 106.3 | 118.8 | 132.6 | 147.5 |
| weight (gr) | | | | | | | |
| Final | | 89.3 | 100.4 | 112.2 | 125.4 | 139.8 | 155.3 |
| weight (g) | | | | | | | |
| Temp. (° C.) | | 10.8 | 10.8 | 10.8 | 10.8 | 10.8 | 10.8 |
| Specific feed | | 1.54 | 1.50 | 1.50 | 1.46 | 1.43 | 1.40 |
| rate (SFR; % | | | | | | | |
| Bw/day) | | | | | | | |
| 110% SFR | | 1.70 | 1.65 | 1.61 | 1.57 | 1.52 | 1.48 |
| Fish | | 2994 | 3331 | 3700 | 4104 | 4541 | 5011 |
| biomass/ | | | | | | | |
| tank (g) | | | | | | | |
| Feed/tank/ | | 46 | 50 | 55 | 60 | 65 | 70 |
| day | | | | | | | |
| (kg) | | | | | | | |
| Feed/fish/day | | 1.2 | 1.3 | 1.4 | 1.5 | 1.7 | 1.8 |
| (g) | | | | | | | |
| Projected | TP1 | 7.56E+07 | 8.19E+07 | 8.82E+07 | 9.45E+07 | 1.07E+08 | 1.13E+08 |
| dose | TP2 | 7.26E+07 | 7.87E+07 | 8.47E+07 | 9.08E+07 | 1.03E+08 | 1.09E+08 |
| (CFU/ | | | | | | | |
| fish/day) | | | | | | | |
| Projected | TP 1 | 8.98E+08 | 8.64E+08 | 8.30E+08 | 7.95E+08 | 8.08E+08 | 7.69E+08 |
| dose | TP 2 | 8.62E+08 | 8.30E+08 | 7.97E+08 | 7.64E+08 | 7.76E+08 | 7.38E+08 |
| (CFU/ | | | | | | | |
| fish | | | | | | | |
| Bw/day; | | | | | | | |
| CFU/kg) | | | | | | | |

A pilot scale mixer will be used for preparation of the 2 test diets (TP1 and TP2) in a 10 kg batch size as per the recipe outlined in Table 28.

Medicated feed will be prepared with two feed pellet sizes: Nutra Supreme 30 and Nutra Supreme 60, which will be delivered according to fish size (Nutra Supreme 30 will be delivered when fish are weighing up to approximately 60 g average Bw, and Nutra Supreme 60, when fish are over 60 g), and will be prepared in equal way. At least three batches are considered to prepare for the whole study period, based on the required feed calibers. Medicated feed will be prepared using the recipe identified in 3 phases as follows:
1—Feed and premix mix for 30 seconds (dry mixing)
2—Fish oil addition over 30 seconds, no more than 0.5% oil
3—Keep mixing for another 60 seconds (wet mixing).

TABLE 28
Test diet recipes, per 10 kg batch size

| | Diet | Feed | | Oil | Batch |
|---|---|---|---|---|---|
| Group | name | (g) | Probiotic premix (g) | (g) | total (g) |
| TP1 | Diet 1 | 9,400 | 500 g (6.29 × 10\hat{ }11 CFUs | 100 | 10,000 |
| | | | total) | | |
| TP2 | Diet 2 | 9,400 | 500 g (6.05 × 10\hat{ }11 CFUs | 100 | 10,000 |
| | | | total) | | |
| NCP | Diet 3 | 9,900 | — | 100 | 10,000 |

### Treatment Administration

During acclimation fish will be fed a commercially available and complete unmedicated commercial salmonid diet of the same pellet size and approximate composition as the negative control diet, provided by Skretting.

During medication period, fish will be fed approximately 110% of the specific feed rate (SFR) identified using a Skretting feed table. Calculations for projected weekly SFR and dose rates over the study period. Minimum daily dose rates (in CFU/fish Bw/day) are projected in Study Weeks 4 and 5. Dose rate calculations consider the assayed CFU/g premix per Table 27 and 28.

Exploratory weight sampling will be performed approximately every 14 days (10 fish per tank) and will be registered. Average body weights will allow adjusting feed amounts to be delivered to each tank and every 7 days feed will be calculated based on projected body weight.

Medicated feed quantity will be calculated for 14 days and thereafter be pre-weighed for assuming a constant daily SFR over the administration period, and stored in labelled plastic bags with date, group and tank identification.

### Body Weight and Length Assessments

Body weight data will be recorded from individual anaesthetized fish on tank set up, SD-1, SD 35 and at the end of study (SD84). Weigh scales will be calibrated on each day prior to use. This information will be recorded. The length (nose to fork length) will be recorded in all sampled fish and survivors.

### Sample Collection Schedule

Over the study period, fish will be sampled for data and tissue collection as Table 28.

TABLE 28
Overview of fish procedures and sampling schedule.

| | Approximate Study Day | | | |
|---|---|---|---|---|
| | −7 | −1 | 35 | 84 |
| Sample type | Tank | Before Start | Medicated feed phase | |
| | set up | Medicated Feed | | |
| | & fish | | | |
| | selection | | | |
| Body weight | ✓ 100% | ✓ 100% fish | ✓ 10 fish | ✓ 100% fish |
| | fish | | per tank | |
| Collection | — | ✓ 2 fish | ✓2 fish | ✓ 4 fish |
| of Tissue | | per tank | per tank | per tank * |
| and Blood | | | | |
| samples | | | | |
| Euthanasia | — | — | — | ✓ 100% fish |

* May be assessed up to two days.

### Assessment of Efficacy

During the study period, in case that any moribund fish reach a humane endpoint (as described below) will be removed from a tank, euthanized, and counted as a mortality. A fish is selected for humane endpoint if any of the two following criteria is observed:
Criterion 1: Fish is in lateral-recumbency, dorsolateral-recumbency, or dorsal-recumbency on the bottom of the tank or floating at the water surface.
Criterion 2: Fish is unable to achieve or maintain a normal orientation for a salmonid in the water column.

Any mortality or terminally moribund fish noticed during the observation period will be removed from the tank; will be recorded on CRF ‘Necropsy’ and assigned an increasing and sequentially numbered case identification number (CID), beginning with ‘1’. Mortalities and moribund fish will be noted as ‘M’.

### Definition of Efficacy

TP efficacy will be defined as meeting the following criteria:
Relative improvement of bodyweight and growth parameters including, but not limited to: Weight Gain Ratio (WGR) and Specific Growth Rate (SGR), of the TP in comparison to the NCP. Calculations may consider the following periods: SD 0 to SD 35, SD 35 to SD 84, SD 0 to SD 84.

### Description of Statistical Methods and Calculations

The tank will be the experimental unit and fish will be the observational unit. Variable calculations and statistical analyses will be performed for individual phases and the overall study. Growth performance variables may include but are not limited to Weight Gain Ratio (WGR) and Specific Growth Rate (SGR). The effect of treatment on growth performance variables will be analyzed, based on data of fish weight, including the mean difference of more than two samples (one control and two treatments) at baseline of the test (time 0) and at the end of the test (Time t) considering time t as a cutoff according to crop densities.

### Example 11

### Genomic and Matabolomic Analysis of Probiotic Lactobacillus Strains

Analysis of each of Lactilactobacillus strains, Lactobacillus curvatus strains ELA204093 (strain 93), ELA204100 (Strain 100) and ELA214388 (strain 388) and Lactilactobacillus sakei strain ELA214391 (strain 391) was conducted. The genome nucleic acid sequence for Lactilactobacillus curvatus strains ELA204093 (strain 93) is provided in SEQ ID NO: 1. The genome nucleic acid sequence for Lactilactobacillus curvatus strains ELA204100 (strain 100) is provided in SEQ ID NO: 2. The genome nucleic acid sequence for Lactilactobacillus curvatus strains ELA214388 (strain 388) is provided in SEQ ID NO: 3. The genome nucleic acid sequence for Lactilactobacillus sakei strain ELA214391 (strain 391) is provided in SEQ ID NO: 4.

Predicted Bacteriocins are provided in Table 29. Also, Strain 391 is predicted to have a TP3KS type metabolite. Genome analysis is provided in Table 30.

TABLE 29

| | | | | Interpro | | |
|---|---|---|---|---|---|---|
| | | Enzy | uniprot | Group | | |
| Qseqid | Sseqid | ID | ID | Name | length | mismatch |
| LAB388_01458 | EN39341087\| | EN39341087 | D4VV13 | N-acetylmuramoyl- | 175 | 61 |
| | D\|D4VV13\|bacteriocin | | | L-alanine amidase | | |
| | | | | domain | | |
| LAB391_01405 | EN74141431\| | EN74141431 | A8E261 | N-acetylmuramoyl- | 193 | 66 |
| | \|A8E261\|Putative | | | L-alanine amidase | | |
| | | | | domain | | |
| LAB391_01405 | EN71201520\| | EN71201520 | GM0020 | N-acetylmuramoyl- | 161 | 41 |
| | \|GM0020\|Lys | | | L-alanine amidase | | |
| | 170-87 | | | domain | | |

TABLE 30

| | Sample Names | | |
|---|---|---|---|
| | LAB388 | LCUR100 | LAB391 |
| Contigs | 100 | 77 | 23 |
| Bases | 1,962,512 | 1,889,821 | 1930890 |
| CDS | 1,967 | 1,873 | 1,952 |
| CRISPR | 4 | 1 | 1 |
| Gene | 2,040 | 1,955 | 2,013 |
| misc_RNA | 36 | 36 | 31 |
| tRNA | 36 | 45 | 29 |
| tmRNA | 1 | 1 | 1 |

### Example 12

### Effect of in-Feed Administration of PTA-16, PTA-17, LCELA388 and LSELA391 on Growth Performance in Atlantic Salmon in Saltwater

### Materials and Methods

The goal of this study was to confirm the efficacy of L. curvatus strains PTA-127116 and PTA-127117 (TP1) in saltwater with longer duration. Two additional strains, L. curvatus LcELA388 and L. sakei LsELA391 (TP2), isolated from North American salmon intestine were also tested in this study. An 11-week study was performed. 450 female Atlantic salmon parr weighing 125-145 g were recruited from Icelandic hatcheries (CIC), distributed without intentional bias into six 500 L study tanks randomly allocated to three groups with two replicate tanks for each group, and acclimatized to the conditions of the feeding trial and fed with a basal commercial diet with composition appropriate for body weight for fourteen days without handling. The control group (NCP-S) was fed commercial extruded basal diet and the probiotic groups were given TP1-S and TP2-S containing a combined dose of 6.05-6.29×10⁷ CFUs/g. Fish were fed approximately 110% of the specific feed rate (SFR, >1.15) using a Skretting feed table using an automatic feeder. The amount of feed delivered ranged from 1.4 to 3.0/kg/tank/week. Over the study period, fish were maintained in 500 L tanks of flow through fresh water under a photoperiod regime of 24-hour day light. Water flow during the holding period and during experimental period was set at a rate to ensure a minimum of 1.0-1.3 total volume water exchange/hour. Supplemental oxygen was delivered as needed to the tank water to maintain appropriate saturated oxygen levels (70-130% saturation) and water temperature for all tanks was monitored daily.

### Results

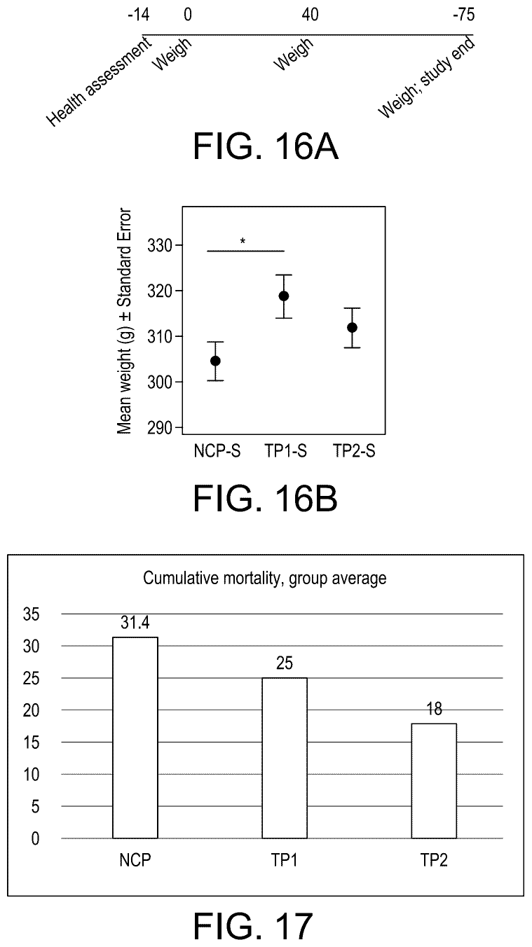

Two test products (TP1-S, TP2-S) and one negative control product (NCP-S) (FIG. 16) were added to commercial fish pellets and fed to three groups of 75 fish each divided between two tanks per group. TP1-S, Test Product 1-Saltwater; TP2-S, Test Product 2-Saltwater; NCP-S, Negative Control Product-Saltwater.

All fish were weighed and returned on study days (SD) 0, 40, and 75 (FIG. 16A). 20 fish were randomly selected from each tank, weighed, and returned on SD 18, 32, and 54. All fish were euthanized at the end of the study on SD 75 (FIG. 16A). While TP2-S(Test Product 2) did not differ significantly from NCP-S, TP1-S weighed significantly more (P=0.041 for NCP-S vs TP1-S, ANOVA with Dunnett's test (n=64)).

Average weights for TP1-S, TP2-S, and NCP-S were 318.7 g, 311.8 g, and 304.5 g, respectively (FIG. 16B). The specific growth rates (SGR) for TP1-S, TP2-S, and NCP-S were 2.23, 2.16 and 2.06, respectively. Weights relative to the control group were 4.7% and 2.4% for TP1-S and TP2-S, respectively. The 4.7% increase in final bodyweight for TP1-S translated to a 7.5% increase in average daily weight gain during the study compared to the control. No gross pathology (data not shown) nor mortality revealed concerns about safety.

### Example 13

### Effect of in-Feed Administration of PTA-16, PTA-17, LCELA388 and LSELA391 on Survival of Atlantic Salmon in the Presence of SRS Challenge

### Materials and Methods

The goal of this study was to evaluate the efficacy of L. curvatus strains PTA-127116, PTA-127117 (TP1) and LcELA388 and L. sakei strain LsELA391 (TP2) for survival in the presence of Piscirickettsia salmonis challenge in a cohabitation model. A 90-day study with the last 61 days taking place during the P. salmonis cohabitation challenge period was performed. 450 female Atlantic salmon parr weighing 139+/−2.6 grams were recruited, distributed without intentional bias into six 500 L study tanks randomly allocated to three groups with two replicate tanks for each group, and acclimatized to the conditions of the feeding trial and fed with a basal commercial diet with composition appropriate for body weight for fourteen days without handling. The control group (NCP-S) was fed commercial extruded basal diet and the probiotic groups were given TP1-S and TP2-S containing a combined dose of 6.05-6.29×10⁷ CFUs/g. Fish were fed approximately 145% of the specific feed rate (SFR, >1.15) using a Skretting feed table using an automatic feeder. The amount of feed delivered ranged from 1.4 to 3.0/kg/tank/week. Over the study period, fish were maintained in 500 L tanks of flow through fresh water under a photoperiod regime of 24-hour day light. Water flow during the holding period and during experimental period was set at a rate to ensure a minimum of 1.0-1.3 total volume water exchange/hour. Supplemental oxygen was delivered as needed to the tank water to maintain appropriate saturated oxygen levels (70-130% saturation) and water temperature for all tanks was monitored daily. One week before challenge started, water temperature was raised in order to acclimate the fish to the challenge water parameters.

The P. salmonis challenge was performed on a cohabitation mode by inserting P. salmonis inoculated fish to act as shedders. On challenge day, shedder fish of the same origin as the test fish were inserted into the six study tanks after injecting them intraperitoneally (i.p.) with a 0.1 ml dose of the challenge inoculum. The inoculum concentration was 3.9×10⁶ colony forming units (CFU)/ml of P. salmonis belonging to the EM90 (A) genogroup. Fifteen shedders were inserted into each study tank, corresponding to a 21.4% of the test fish number (N=70). Challenge lasted for 61 days.

### Results

All shedder fish died within the study period. At the end of the challenge period, cumulative mortality within the experimental groups TP1 (L. curvatus strains PTA-127116 and PTA-127117) and TP2 (L. curvatus strain LcELA388 and L. sakei strain LsELA391) was 25.0±9.1% and 18.0±0.8%, respectively. Control group cumulative mortality was 31.4±2.0%. See FIG. 17. Statistically significant differences on survival analyses were found between the control and TP2 groups.

### Example 14

### Effect of Dietary Supplementation of Probiotics During Post-Smolt Stage (Sea Water), on Growth Performance, Immune Response, and Antioxidant Properties of Atlantic Salmon (Study No.: VCC-0136)

The main objectives of the study are to evaluate growth and performance in post-smolts of Atlantic salmon fed experimental diets with probiotics compared to a negative control and between groups. The secondary objectives of the study are to sample fish for characterizing immune response and blood chemistry and to compare to negative control and between groups. Also studied is preliminary stability of the IVPs following preparation of experimental feeds.

### Study Schedule

TABLE 31
Study schedule

| | | Study |
|---|---|---|
| Task | Activity | week |
| 1 | Fish distribution into study tanks, | −3 |
| | acclimation (CR#1) | |
| 2 | Initial sampling (S1) | −1 |
| 3 | Feed production and stability | −1/0 |
| | sampling FEED | |
| | #1, #2; #3 and #4 | |
| 4 | Feeding experimental feeds | 0-10 |
| | (probiotics), for a 10-week period | |
| 5 | Fish weight monitoring | 2-10 |
| 6 | Intermediate sampling (S2) | 5 |
| 7 | Final sampling (S3) | 10 |
| 8 | Study termination | 10 |
| 9 | Report | 14 |

### Day 0 Definition

The day of start of feeding is defined as day 0.

### Study Summary

This study was designed to assess in vivo growth performance in Atlantic salmon fed with test substances (TP: Probiotic 1 and Probiotic 2). The study was comprised by two treatment groups (TP: Probiotic 1-P1 and Probiotic 2-P2) and one control group (NCP) in duplicate study tanks.

Fish were acclimated for fourteen days (14) before commencement of feeding with the experimental feeds. On day of acclimation/distribution, fish were sedated with Isoeugenol and anaesthetized with Benzocaine, individual weight/length of all fish were recorded, and fish were distributed without bias to their study tanks.

An Initial weight/length measure was performed at distribution (week-3) with an average weight of 128.3 g (SD±7.4). Initial sampling (S1) was carried out in week-1 with an average weight of 143.5 g (SD±9.9).

On study day 0 (start of feeding), fish were fed only with the study diets (test or control) for the following 10 weeks of feeding. Four (4) study diets batches were prepared using a non-coated dry commercial feed pellet where the probiotics were suspended in fish oil mix manually and by using a semi automatic dispersion tool, then feed pellets were coated with the oil by a Forberg vacuum coater. At day of preparation samples were collected for probiotic stability evaluation, and later every 14 days after each batch preparation.

Intermediate sampling (S2) was carried out after 5 weeks of feeding showing an average weight of 240.1 g, with differences among groups, and the final sampling was performed after 10 weeks of feeding with an average weight of 311.6 g showing differences among groups (S3). Fish weights were recorded every two weeks, and feed were sampled for stability every 14 days S1, S2 and S3 include 100% of fish weighted and length measured.

At all every sampling point blood/serum was collected as well as gut content and stored at −80° C. Additionally pyloric caeca was collected and stored in RNA later at −20° C.; distal intestine was fixed in 4% buffered formalin and stored at ambient temperature. Head kidney smear were collected for immune marker analysis.

In relation to the growth performance parameters: The fish weight increased from an average range 143-146 g to a range of 303-334 g during the study period. There were no significant differences in Final Weight (FW), Fork Length (FL), Specific Growth Rate (SGR), Thermal Growth Coefficient (TGC) and Weight Gain (WG) of the diet groups from the initial (S1) to the intermediate (S2) sampling. On the other hand, the Condition Factor (CF) were significantly impacted by the diet groups; Control group showed lower CF in comparison to P1.

Additionally, there were no significant differences from the initial (S1) to final sampling (S3) with respect to FL, SGR, TGC and WG. Differences were observed on FW, where the Control group showed lower in comparison to P1, with no differences with P2. Differences was detected also on CF, Control group showed lower CF when compared to P1 group.

### Study Compounds

### Test Substance

The test substance(s) were received, labelled and stored. The test substances were kept refrigerated at 5±1.5° C. and protected from light.

$$TP 1 = Lactobacillus curvatus \#93 + Lactobacillus curvatus \#100 ( two lots )$$

$$TP 2 = Lactobacillus curvatus \#388 + Lactobacillus curvatus \#391 ( two lots )$$

### Control Substance/Control Feed

There was no added control substance. Oil mix and inclusion level was the same among experimental feeds batches (i.e., 18%).

Test substances were combined with a standard diet to form the test products.

### Experimental Fish and Rearing Conditions

TABLE 32
Test fish

| | |
|---|---|
| Species | Atlantic salmon (Salmo salar), post-smolts |
| Strain | Stofnfiskur |
| Origin | Iceland |
| Gender distribution | All females |
| Batch No. | ST2006 (Acclimation JNo. 2114) |
| Fish size | 128,3 g (SD ± 7.4) |
| Treatment status | Non-vaccinated |
| Physiological status | Post-smolt |
| Number of fish | 420 test fish + 30 extra fish (samples) |

### Inclusion/Exclusion (Non-Inclusion) Criteria

### Inclusion Criteria:

All study fish participating in the study were unvaccinated. At first, the infectious status of the study fish and/or egg origin was documented to be negative for the following pathogens: Piscirickettsia salmonis, Renibacterium salmoninarum, Flavobacterium psychrophilum, Piscine orthoreovirus (PRV), Infectious Salmonid Anemia Virus (ISAv) and Infectious Pancreatic Necrosis virus (IPNv). Fish batches are screened by q-PCR for the above-mentioned pathogens before smolt release from hatchery to challenge rooms. Samples were obtained between 14 days before the acclimation period started. A new sampling was performed at the hatchery, confirming the PRV positiveness of the sampled tank.

Fish batch was assessed for gill Na+/K+ ATPase values at an external laboratory during the fourth week of 24:0 h photomanipulation. Fish were deemed smoltified when ATPase values are ≥15 U/mg.

All study fish in the study were never treated with antibiotics and were deemed clinically healthy, meaning they had no condition causing an impediment to mobility or feeding, i.e., fish had good quality fins and good body condition provided they were selected during the study tanks conformation.

### Exclusion (Non-Inclusion) Criteria

Sexually matured, injured or deformed fish and fish that appeared not to be fully smoltified were excluded from the study upon arrival or during distribution or at treatment in study tanks. As a provision any fish batch that could have gone through an outbreak or had been detected with pathogens was excluded. PRV was an exemption, which was tested to know the sanitary status and prevalence.

### Husbandry Management

Fish and tanks were tended and monitored on a daily basis. Fish were fed via automatic feeders (115 EGI, EWOS Growth Index) using feed provided by CIC. Salinity was ca. 27% % during the whole study. The photoperiod light:dark was 24:0 h the whole study. Environmental parameters were recorded automatically (water exchange, temperature and oxygen saturation inside each tank; salinity and pH in header tank).

TABLE 33
Husbandry conditions

| | |
|---|---|
| Tank size | 0.5 m³ (500 l) |
| Salinity | Average of 27.3‰ (SD ± 0.81) during acclimation. |
| | Average of 27.5‰ (SD ± 0.51) during the experimental feeding |
| Density | About 20.5 kg/ m³ at stocking: |
| | About 40.5 kg/ m³ at termination (differences depending on the |
| | experimental group) |
| Temperature (range) | 12.3° C. (SD ± 0.18) during acclimation |
| | 12.3° C. (SD ± 0.14) during experimental feeding period |
| Dissolved oxygen | >70% saturation inside tanks |
| Flow | 1.0-1.3 tanks turnover/h during the study |
| Water discharge | Tube overflow system |
| Cleaning | Once a day (flushing only if required) |
| Photoperiod regime | L:D = 24:0 |
| Feeding | Arvotec automatic feeder). Microration feeding from 16:00 to 04:00 |
| | (approx. 96 pulses in 12 h). |

TABLE 34
Animal Welfare

| Topic | Indication |
|---|---|
| Justification of number of fish to be | 75 fish per tank, 450 total fish, are required to have |
| used: | sufficient data for growth evaluation and sufficient fish |
| | for samplings. |
| | Two replica per group. |
| Endpoint definition | Sampling at termination |
| Temperature adjustments | Standard is no more than 1° C./day |
| Salinity adjustments | approx. 28‰ is required for keep post-smolts of salmon |
| Anaesthesia | Benzocaine for weight records; Isoeugenol |
| | before fish movements or handling as sedative |
| Euthanasia | Overdose of benzocaine |
| Starvation | 8 h before weight samplings (SD 36 and SD71) and 60% |
| | of the ration the day before |

### Study Design

### Design Summary

This study was designed to assess in vivo growth performance in Atlantic salmon fed with test substances (TP: Probiotic 1 and Probiotic 2). The study comprised two treatment groups (Probiotic 1 and Probiotic 2; P1 and P2) and one control group (NCP) in duplicate study tanks. Fish weight was recorded during the study. Tissue and blood samples were collected during the study.

Fish were acclimated for fourteen days before commencement of feeding with the experimental feeds (see Table 36). On day of acclimation/distribution, fish were sedated with Isoeugenol and anaesthetized with Benzocaine, individual weight/length of all fish was recorded, and fish were distributed without bias to their study tanks. On study day 0 (start of feeding), fish were fed only with the study diets (test or control) for the following 10 weeks of feeding. Four (4) study diets batches were prepared using a non-coated dry commercial feed pellet where the probiotics were suspended in fish oil mix manually and by using a dispersion tool, then feed pellets were coated with the oil by a Forberg vacuum coater. At day of preparation samples were collected for probiotic stability evaluation, and later every 14 days after each batch preparation.

Prior samplings, all study fish were sedated with Isoeugenol in the study tank. Then fish were anaesthetized with benzocaine for distribution and weight samplings. Before tissue and blood sampling, fish were euthanized with Benzocaine. Blood, tissue samples and intestine content were sampled before feeding and two times during the study.

### Day 0 Definition

The day of start of feeding was defined as study Day 0.

### Marking

There was no marking procedure in the study fish.

### Feeding and Starving of Fish

Fish were fed (≥1.2 specific feeding rate) during the feeding period. Throughout the duration of the study the fish were overfed to ensure that access to feed is not limited. All the fish were individually weighed and transferred into the study tanks. All study tanks were fed by automatic feeders. Feeding rates were adjusted to ensure overfeeding—not heavy overfeeding that may clog the tanks and make for sub-optimal tank environment. Fish in all tanks were starved as shown in Table 35.

TABLE 35
Times of starving/feed deprivation
of fish before and during the study

| Fish group starved | Event |
|---|---|
| All fish | 24 h before distribution |
| All fish | 8 h before defined weight samplings (SD 36 |
| | and SD 71) and 60% of the ration |

### Test Groups

TABLE 36
Allocation of fish groups during feeding period and samplings.

| | Test/ | Tank | | Sampled | | |
|---|---|---|---|---|---|---|
| Tank | control | volume | Fish | fish (no) | Density | Salinity |
| ID | substance | (L) | (N) | per tank | (kg/m³) ¹ | (‰) |
| B101 | Probiotic 1 | 500 | 75 | S1: 5 | 20.4 | 27-28 |
| | (P1) | | | S2: 5 | | |
| B102 | Probiotic 1 | 500 | 75 | S3: 10 | 20.6 | 27-28 |
| | (P1) | | | | | |
| B103 | Control (Co) | 500 | 75 | | 20.5 | 27-28 |
| B106 | Control (Co) | 500 | 75 | | 20.5 | 27-28 |
| B104 | Probiotic 2 | 500 | 75 | | 20.4 | 27-28 |
| | (P2) | | | | | |
| B105 | Probiotic 2 | 500 | 75 | | 20.7 | 27-28 |
| | (P2) | | | | | |

### Feed Preparation

Study diets were prepared using a non-coated dry commercial feed pellet with a size of 4 mm (EWOS Micro 100) and/or 6 mm (Micro 250). Test diets (P1, P2, and NCP) were prepared as follows: the test substances, Probiotic 1 or Probiotic 2, were suspended in an oil mix (blend of fish oils, blend of vegetable/poultry/marine oils, and/or soy lecithin) by using Ultraturrax T50 (with a dispersion tool); then feed pellets will be coated with the blended mix oil by a Forberg vacuum coater. Temperature in oil mix, non-coated dry feed pellets and during the dispersion and coating process was kept below 35° C. Test diets (TP1, TP2) were prepared to reach 5% of Probiotic 1 and Probiotic 2. Control diet (NCP) was coated with same oil-mix and at same inclusion level. Study diets were coated with 18% weight-based oil-mix. The study diets were given from the day of commencement of the feeding and throughout the whole-study period. Feed production was documented in a medicated feed preparation record form.

Feeds were given during the feeding period, except for day of starving/feed deprivation/reduced feeding prior samplings.

Experimental feeds were produced according to the projection of fish weight and required amounts defined by specific feeding rations, daily delivered feed was documented. Five (5) time points of feed preparations (FEED #1 to #5) were programmed to prepare in total 15 batches of experimental feeds for each of the experimental diets (Table 37). Only 12 batches were used in this present study.

TABLE 37
Feed batches prepared of each experimental diet.

| | | Amount | |
|---|---|---|---|
| | Pellet | prepared | Probiotic Lot. |
| Preparations | size | (Kg) | No. |
| FEED #1 | 4 mm | 10 | — |
| | | 10 | Chinook 2-1 |
| | | 10 | Chinook 2-2 |
| FEED #2 | 4 mm | 10 | — |
| | | 10 | Chinook 2-1 |
| | | 10 | Chinook 2-2 |
| FEED #3 | 4 mm | 5 | — |
| | | 5 | Chinook 2-1 |
| | | 5 | Chinook 2-2 |
| FEED #4 | 6 mm | 10 | — |
| | | 10 | Chinook 3-1 |
| | | 10 | Chinook 3-2 |
| FEED #5* | 6 mm | 2 | |
| | | 2 | Chinook 3-1 |
| | | 2 | Chinook 3-2 |

*Not used

### Outcome Parameters—Measures of Effect—Growth Performance Calculations

Fish growth is the main outcome parameter in the study. Samples and registration of sampled fish is also an outcome parameter in the study. Fish growth performance was analysed using the following equations:

$$Weight gain ( W G \% ) = ( ( FW - IW ) / IW ) \times 100$$

$$Specific growth rate ( S G R ) = ( ( Ln ( FW - ( IW ) ) / D ) \times 100$$

$$Thermal growth coefficient ( T G C ) = ( ( {( FW )}^{( 1 / 3 )} - {( IW )}^{( 1 / 3 )} ) / T \times D ) ) \times 200$$

$$Condition factor ( C F ) = ( FW / {FL}^{3} ) \times 100$$

Where, FW=mean final body weight of fish (g), IW=mean initial body weight of fish (g), T is water temperature in ° C., D is feeding duration in days. IL and FL are the initial and final fork length (cm) of fish, respectively.

### Statistical Analysis

Statistical analyses were performed. Data was analysed for descriptive statistics and significant differences among groups by GraphPad Prism 7 (version 9.31, GraphPad Software, Inc.). Tanks and/or treatment/groups were used for the analysis of growth performance. Normality of the data was checked with various tests within GraphPad Prism 7. One- or Two-way ANOVA (parametric or non-parametric) with Multiple comparisons tests were run depending on the characteristics of the data.

No Adverse Events were registered in this study.

### Sampling and Diagnostics

### Weight

Calculation of mean fish weight at distribution, on sampled fish and at termination was carried out. All study fish were weighed and measured at distribution, S1 (initial sampling), at S2 (intermediate sampling) and at termination S3. Additionally, weight sampling of 20 fish per tank was programmed every two weeks.

TABLE 38
Summary of activities and samplings of the Study.

| Study Day +/− 3 | Study Activity |
|---|---|
| −21 +/− 2 − 18 | Health assessment / Sample for screening |
| | 450 fish were enrolled and distributed in 6 500 L tanks (total 75 fish per |
| | tank). All fish were individually weighed (100% population) and length |
| | measured. |
| −18 to 0 | Acclimation; Randomization. |
| −1 | Si: sampling 1 |
| | Weight sampling: all fish were individually weighed (100% |
| | population). |
| | Sample collection: 10 fish per treatment (5 per tank) were |
| | euthanized and sampled (blood and gut samples) |
| 0-71 | Record daily observations. |
| | Sample weight each two weeks for feeding rate calculation (20 fish |
| | per tank). |
| | Study Diets were delivered from SD 0 until end of trial. |
| | Feed samples for stability check, every 14 days. |
| 36 | S2: sampling 2 |
| | Weight sampling: all fish were individually weighed (100% population) |
| | Sample collection: 10 fish per treatment (5 per tank) were |
| | euthanized and sampled (blood and gut samples). |
| 71 | S3: sampling 3 |
| | Weight sampling: all fish were individually weighed (100% population) |
| | Sample collection: 20 fish per treatment (10 per tank) were |
| | euthanized and sampled (blood and gut samples). |

### Feed Sampling and IVP Stability

Study feeds were sampled at day of preparation and every 14 days. Feed sampling was performed. A protocol used to assess IVP stability i.e., CFU counts.

Feed was processed and CFU counts were performed as follows. About 10 g feed was weight and placed in 50 ml tube. 20 mL sterile PBS was added into the 50 ml tub. The mixture was soaked at least 30 minutes and vortexed well. 4500 μl sterile PBS was poured into four 15 ml centrifuge tubes, and labelled label 1-4. 500 μl of supernatant soaked mixture was added to tube 1 (10⁻¹ dilution), and pulsed vortex for 10 seconds. Proceeded with serial dilutions until 10⁻⁴, repeating vortex every time. Each dilution (100 μl) was plated in duplicate in Man, Rogosa and Sharpe (MRS) agar and spread evenly using a Drigalsky loop (plate 100 μl sterile PBS as a blank control). Procedure was repeated with the untreated feed for a negative feed control. Plates were incubated at 22 (+/−1° C.) for 4 (+/−1)-4 days. After final day of incubation each plate was photograph and afterwards colonies counted using ImageJ. CFU was calculated based on colonies per dilution and plotted on log curve. The procedure started with the control diet to secure no cross contamination.

### Results

### Observations

1) Environmental parameters including oxygen saturation, temperature, salinity, pH, water exchange and feed amounts were recorded during acclimation and feeding periods.
2) All fish that die during acclimation (minimum 5 days before study start) and after study start—or are removed at humane endpoints—were necropsied.
3) Weight and fork length were recorded in each sampling.

### Efficacy Assessment—Growth Performance

Weight Gain, Specific Growth Rate (SGR), Thermal Growth Coefficient (TGC) and Condition Factor (CF) of the TP (P1 and P2) in comparison to the NCP (Control), were calculated between the period of initial sampling and intermediate sampling (S1 to S2); and initial sampling and final sampling (S1 to S3). There were no mortalities during the study. The growth performance parameters are presented in Table 39.

TABLE 39
Growth performance indicators of Atlantic salmon offered experimental feeds: Control
(NCP), Probiotic 1 (P1) and Probiotic 2 (P2), evaluated at different time periods.
Initial to Intermediate sampling

| Parameters | [symbol] | [symbol] | [symbol] | FL (cm) | CF (g/cm3) | SGR | TGC | WG (%) |
|---|---|---|---|---|---|---|---|---|
| [symbol] of | | | | | | | | |
| main effects | | | | | | | | |
| Control [symbol] | 145.07 ± | 23.39 ± | 236.70 ± | 26.75 ± | 1.23 ± | 1.36 ± | 2.10 ± | 63.20 ± |
| | 0.83 | 0.05 | 1.9 | 0.07 | 0.00 [symbol] | 0.04 | 0.06 | 2.10 |
| Probiotic | 142.78 ± | 23.36 ± | 242.58 ± | 26.93 ± | 1.24 ± | 1.48 ± | 2.28 ± | 70.00 ± |
| 1 (P1) | 0.89 | 0.05 | 2.51 | 0.09 | 0.00 [symbol] | 0.07 | 0.12 | 4.10 |
| Probiotic | 142.65 ± | 23.37 ± | 240.93 ± | 26.73 ± | 1.26 ± | 1.45 ± | 2.27 ± | 68.45 ± |
| 2 (P2) | 0.80 | 0.04 | 2.35 | 0.08 | 0.00 [symbol] | 0.00 | 0.01 | 0.15 |
| P values | 0.103 | 0.916 | 0.103 | 0.1543 | 0.013 | 0.200 | 0.330 | 0.170 |

Initial to Final sampling

| | [symbol] | R (cm) | [symbol] | FL (cm) | CF (g/cm3) | | | |
|---|---|---|---|---|---|---|---|---|
| Parameters | SEM | SEM | SEM | [symbol] | [symbol] | SGR | TGC | WG (%) |
| [symbol] of | | | | | | | | |
| main effects | | | | | | | | |
| Control [symbol] | 145.07 ± | 23.39 ± | 304.47 ± | 29.37 ± | 1.19 ± | 2.06 ± | 1.69 ± | 109.90 ± |
| | 0.83 | 0.05 | 4.23 [symbol] | 0.11 | 0.00 [symbol] | 0.03 | 0.02 | 2.00 |
| Probiotic | 142.78 ± | 23.36 ± | 318.69 ± | 29.64 ± | 1.21 ± | 2.23 ± | 1.84 ± | 123.30 ± |
| 1 (P1) | 0.83 | 0.05 | 4.73 [symbol] | 0.13 | 0.00 [symbol] | 0.14 | 0.13 | 10.90 |
| Probiotic | 142.65 ± | 23.37 ± | 311.75 ± | 29.62 ± | 1.19 ± | 2.17 ± | 1.78 ± | 117.95 ± |
| 2 (P2) | 0.80 | 0.04 | 4.31 [symbol] | 0.11 | 0.00 [symbol] | 0.04 | 0.03 | 3.05 |
| P values | 0.103 | 0.916 | 0.033 | 0.197 | 0.024 | 0.467 | 0.467 | 0.333 |

[symbol]

[symbol]

indicates data missing or illegible when filed

The fish weight increased from an average range 143-146 g to a range of 303-334 g during the study period. There were no significant differences in FW, FL, SGR, TGC and WG of the diet groups from the initial (S1) to the intermediate (S2) sampling. On the other hand, CF were significantly impacted by the diet groups; Control group showed lower CF in comparison to P2.

Additionally, there were no significant differences from the initial (S1) to final sampling (S3) with respect to FL, SGR, TGC and WG. Differences were observed on FW, where the Control group showed lower in comparison to P1 (304 g vs. 318 g), with no differences with P2 (311 g). Differences were detected also on CF, Control group showed lower CF when compared to P1 group.

### Stability Assessment of Probiotics in Feed

Five (5) time points of feed preparations (FEED #1 to #5) (Table 37) were prepared accounting for a total of 15 batches of experimental feeds. Only 12 batches were used in the present study; all were tested for stability. At the day of preparation, two samples of about 100 g were collected per experimental diet, and stored at 4° C. These samples were used to monitor the stability of probiotics the next day of preparation of the medicated feeds. Thereafter, the samples were collected directly from the bag stored at the feed storage room (15-20° C.). Exception was the second monitoring (14 days after preparation of feed) which was collected from storage at 4° C. Stability of probiotics in feed was monitored 1 day after preparation (storage at 4° C.), 14 days and 28 days after preparation (storage at 15-20° C.).

Stability of probiotics in feed are graphically shown in the following Table 40.

| FEED | Batch | | Calculated concentration (CFU / ml) | | |
|---|---|---|---|---|---|
| # | JNo. | Product | Day 1 | Day 14 | Day 28 |
| 1 | 2107 | Control | 6.50, E+02 | 1.65, E+03 | 5.00, E+04 |
| | 2108 | Chinook | 3.60, E+07 | 3.55, E+07 | 3.42, E+07 |
| | | 2-1 | | | |
| | 2109 | Chinook | 2.84, E+07 | 2.53, E+07 | 9.00, E+06 |
| | | 2-2 | | | |
| 2 | 2110 | Control | 1.50, E+03 | 2.80, E+03 | 2.50, E+03 |
| | 2111 | Chinook | 4.25, E+07 | 3.67, E+07 | 3.10, E+07 |
| | | 2-1 | | | |
| | 2112 | Chinook | 2.33, E+07 | 1.11, E+07 | 5.25, E+06 |
| | | 2-2 | | | |
| 3 | 2113 | Control | 1.70, E+03 | 1.30, E+04 | 9.50, E+03 |
| | 2114 | Chinook | 2.40, E+07 | 3.73, E+07 | 2.03, E+07 |
| | | 2-1 | | | |
| | 2115 | Chinook | 1.10, E+07 | 1.00, E+07 | 5.55, E+06 |
| | | 2-2 | | | |
| 4 | 2116 | Control | 0.00, E+00 | 5.00, E+02 | * |
| | 2117 | Chinook | 2.08, E+07 | 1.80, E+06 | * |
| | | 31 | | | |
| | 2118 | Chinook | 5.74, E+07 | 1.85, E+06 | * |
| | | 3-2 | | | |
| 5** | 2119 | Control | 0.00, E+00 | | |
| | 2120 | Chinook | 2.47, E+07 | | |
| | | 3-1 | | | |
| | 2121 | Chinook | 5.94, E+07 | | |
| | | 3-2 | | | |

### Example 15

### Effect of Dietary Supplementation of Probiotics During Post-Smolt Stage (Seawater) on Growth Performance, Immune Response, Antioxidant Properties and Survival of Atlantic Salmon (Salmo salar) in the Presence of a Piscirickettsia salmonis Challenge (Study No.: VCC-0137)

One of the main objectives of the study are to evaluate growth and performance in post-smolts of Atlantic salmon fed experimental diets with probiotics compared to a negative control and between groups. Another main objective is to evaluate the survival rate of the experimental groups in response to a controlled P. salmonis infection challenge under a cohabitation model. Another main objective is to evaluate the infectious status of fish within the experimental groups in response to a controlled P. salmonis infection challenge under a cohabitation model.

### Study Schedule

TABLE 41
Study schedule. The first day of control/experimental
feeding was defined as Study Day 0.

| | | Study |
|---|---|---|
| Task | Activity | day |
| 1 | Health assessment / Samples for screening | −22 |
| 2 | Fish distribution into study tanks | −21 |
| 3 | Formal acclimation begins | −14 |
| 4 | Production of feed batch N1 | −4 |
| 5 | Sampling S1 | −1 |
| 6 | Experimental feeding begins | 0 |
| 7 | Weight sampling (study day 14) | 14 |
| 8 | Production of feed batch N2 | 22 |
| 9 | Water temperature to 15° C. | 23 |
| 10 | Sampling S2 | 28 |
| 11 | Cohabitation challenge; shedders inoculation | 29 |
| 12 | Production of feed batch N3 | 43 |
| 13 | Production of feed batch N4 | 62 |
| 14 | Production of feed batch N5 | 77 |
| 15 | Sampling S3 & Study termination | 90-91 |
| 16 | Final Report | 148 |

### Study Summary

The study aimed at evaluating the short-term performance effects as well as the survival response against a controlled cohabitation P. salmonis infection model of adding mixtures of probiotics (test products) in the feed of Atlantic salmon post smolts. Test product diets (TP) and a control diet were tested under a duplicated tanks design. Study fish were stocked into six 500 L tanks (N=82 fish per tank) and acclimated for 14 days at a water temperature of 12.4±0.2° C. and a salinity of 28.7±2.8%. After the acclimation period, the experimental/control feed delivery started on all tanks in a duplicated tanks design. Average fish weight at this point was 139.5±2.6 grams. Control feed, TP1 (Chinook 3-1), TP2 (Chinook 3-2), TP1 (Chinook 4-1), TP2 (Chinook 4-2) were delivered into tanks. The experimental and control feed were produced on-site and the viability of the added probiotics in these was assessed three times (days 1, 14 and 28 post-production) for each feed batch produced (N=5). The experimental feeding period lasted for 90 days, with the last 61 days taking place during the P. salmonis cohabitation challenge period. During all this period, feed was delivered in excess to all six study tanks with SFR being above 1.45% throughout the whole study. One week before challenge started, water temperature was raised in order to acclimate the fish to challenge water parameters. During this week and during challenge, average temperature was 15.1±0.2° C. and salinity was 27.6±2.2% %. No significant differences were found between study groups when comparing weight gain and other performance parameters for any of the studied periods,

The P. salmonis challenge was performed on a cohabitation mode by inserting P. salmonis inoculated fish to act as shedders. On challenge day, shedder fish of the same origin as the test fish were inserted into the six study tanks after injecting them intraperitoneally (i.p.) with a 0.1 ml dose of the challenge inoculum. The inoculum concentration was 3.9×10⁶ colony forming units (CFU)/ml of P. salmonis belonging to the EM90 (A) genogroup. Fifteen shedders were inserted into each study tank, corresponding to a 21.4% of the test fish number (N=70). Challenge lasted for 61 days. All shedder fish died within the study period. At the end of the challenge period, cumulative mortality within the experimental groups TP1 and TP2 was 25.0±9.1% and 18.0±0.8%, respectively. Control group cumulative mortality was 31.4±2.0%. Statistically significant differences on survival analyses were found between the control and TP2 groups.

All mortalities during the challenge as well as all survivors at the end were examined by necropsy for Piscirickettsiosis clinical signs. During the study, three samplings were performed on day-1 (S1; before experimental feeding started), day 28 (S2; before disease challenge started) and day 90 (S3; end of challenge period and study termination).

### Study Compounds

### Test Substances/Products/Investigational Veterinary Product (IVP)

The test substances were kept refrigerated at about 5.0° C. and protected from light. Test products are as follows:

$$TP 1 = Lactobacillus curvatus \#93 + Lactobacillus curvatus \#100 ( two lots )$$

$$TP 2 = Lactobacillus curvatus \#388 + Lactobacillus curvatus \#391 ( two lots )$$

The test substances were combined with a standard diet to form the test products.

### Control Substance/Product

There was no added control substance.

### Experimental Fish and Rearing Conditions

TABLE 42
Test fish

| | |
|---|---|
| Species | Atlantic salmon (Salmo salar), post-smolts |
| Strain | Stofnfiskur |
| Origin | Iceland |
| Gender distribution | All females |
| Fish size | 114.4 ± 8.5 at stocking |
| Treatment status | Non-vaccinated |
| Physiological status | Post-smolt |
| Number of fish | Total of 602 fish |
| | 492 test fish |
| | 90 shedder fish |
| | 20 fish initial screening |

### Inclusion/Exclusion (Non-Inclusion) Criteria

### Inclusion Criteria:

All study fish participating in the study were unvaccinated. The infectious status of the study fish and/or egg origin is documented to be negative for the following pathogens: P. salmonis, Renibacterium salmoninarum, Flavobacterium psychrophilum, Piscine orthoreovirus (PRV), Infectious Salmonid Anemia Virus (ISAv) and Infectious Pancreatic Necrosis virus (IPNv). Study fish batch was screened by q-PCR for the above-mentioned pathogens before smolt release from hatchery/smoltification room to challenge room. All analyses resulted negative for the following pathogens: R. salmoninarum (PCR and IFAT); P. salmonis (PCR), F. psychrophilum (PCR), PRV and IPNV.

Fish batch was assessed for gill Na+/K+ ATPase values at an external laboratory during the fourth week of 24:0 h photomanipulation; that is 28 days before stocking fish into the study tanks. Fish were deemed smoltified with ATPase values ≥14.5 U/mg.

All study fish were never treated with antibiotics and were deemed clinically healthy, meaning they had no condition causing an impediment to mobility or feeding, i.e., fish had good quality fins and good body condition provided they were selected during the study tanks formation.

### Exclusion (Non-Inclusion) Criteria

Injured or deformed fish and fish that appeared not to be fully smoltified were excluded from the study upon arrival or during distribution or during samplings.

### Husbandry Management

Fish were fed via automatic feeders (115 EGI, EWOS Growth Index) using feed provided by CIC. Salinity was about 28% % during the whole study. The photoperiod light: dark was 24:0 h the whole study. Environmental parameters were recorded automatically (water exchange, temperature and oxygen saturation inside each tank; salinity and pH in header tank).

TABLE 43
Husbandry conditions

| Tanks volume | 0.5 m³ (500 l) |
|---|---|
| Salinity | 28.0 ± 2.6‰ |
| Density | Av. 18.8 kg/ m³ at stocking |
| | Av. 21.5 kg/ m³ at S1 (one day before experimental |
| | feeding started) Av. 29.6 kg/ m³ at S2 (one day |
| | before cohabitation challenge) |
| Temperature | 12.5 ± 0.3° C. during acclimation and for the first 21 days of |
| | experimental feeding |
| | 15.1 ± 0.2° C. seven days before challenge and during challenge |
| Dissolved oxygen | >77% saturation inside tanks (minimum value 77%-maximum value |
| | 118%) |
| Flow | 8.3 ± 0.2 L/min (i.e., 1.0 tanks turnover/h) |
| Water discharge | Tube overflow system |
| Cleaning | Once a day (flushing only if required) |
| Photoperiod regime | L:D = 24:0 |
| Feeding | 115% (automatic feeder). Microration feeding from 16:00 to 04:00 |
| | (approx. 96 pulses in 12 h). SFR average of 1.54 ± 0.1% during |
| | acclimation and first 28 days of feeding. SFR average of 1.45 ± |
| | 0.1%. |

TABLE 44
Animal Welfare

| Topic | Indication |
|---|---|
| Justification of number | 82 test fish per tank, 492 total test fish, are |
| of fish to be used: | required to have sufficient data for growth |
| | evaluation, samplings and for survival |
| | evaluation on the challenge test. Every group |
| | will have only two replicas. Furthermore, 15 |
| | shedder fish per tank will be used for the |
| | cohabitation challenge (total of 90 shedders) |
| Endpoint definition | Cumulative mortality. Sampling at termination |
| Temperature adjustments | Standard is no more than 1.5° C./day |
| Salinity adjustments | >27‰ is required for keeping post-smolts of |
| | salmon |
| Anaesthesia | Benzocaine for weight recording and before |
| | shedder fish i.p. (intraperitoneal) inoculation |
| Euthanasia | Overdose of benzocaine |
| Starvation | 24 h before fish stocking and before final |
| | euthanasia of challenge survivors |

### Study Design

### Study Aims and Study Groups

This study was designed to assess the effects of different test products (Probiotic 1-TP1- and Probiotic 2-TP2-) tested in duplicated tanks on growth performance and in response to a P. salmonis challenge when fed to Atlantic salmon post-smolts. The study also includes a control group in duplicate study tanks (Non-Treated Control NCP). See Table 45 for study groups distribution and tanks assignations.

TABLE 45
Allocation of fish groups during feeding period and fish destination.
Fish were stocked in a layered distribution in groups of 20
at a time from tank B101 until B106. Then the process was repeated
until reaching the desired number of 82/tank.

| | | | | | Fish for | |
|---|---|---|---|---|---|---|
| | Test/ | Experimental | Tank | Fish | samplings | |
| Tank | control | group | volume | (N) | (S1-S2- | Fish for |
| ID | substance | ID | (L) | * | S3) N | challenge |
| B103 | None | NCP | 500 | 82 | 5- | 70 |
| | (control | | | | 5- | |
| | group) | | | | 10 | |
| B106 | None | NCP | 500 | 82 | 5- | 70 |
| | (control | | | | 5- | |
| | group) | | | | 10 | |
| B101 | Test | TP1 | 500 | 82 | 5- | 70 |
| | Product 1 | | | | 5- | |
| | | | | | 10 | |
| B102 | Test | TP1 | 500 | 82 | 5- | 70 |
| | Product 1 | | | | 5- | |
| | | | | | 10 | |
| B104 | Test | TP2 | 500 | 82 | 5- | 70 |
| | Product 2 | | | | 5- | |
| | | | | | 10 | |
| B105 | Test | TP2 | 500 | 82 | 5- | 70 |
| | Product 2 | | | | 5- | |
| | | | | | 10 | |

* Two extra fish were included from the beginning on each study tank to compensate in case of eventual mortalities during this period. These extra fish were discarded from the study during S2, right before challenge (see section 9.6 for samplings details).
**S3 took place at the end of the challenge period, so those 10 fish are also included in the “Fish for challenge” column.

### Day 0 Definition

The first day of experimental/control feed delivery to the study groups was defined as study Day 0.

### Study Duration

Fish were stocked into the study tanks on study day-21. Formal study acclimation period started on study day-14. Experimental/control feeding started on study day 0) and lasted for a period of 90 days. Of these, the first 29 days took place without any challenge while during the latter 61 days the experimental feeding took place concomitantly with a cohabitation challenge with P. salmonis.

### Experimental/Control Feeds Preparation and Delivery

Study diets were prepared at the experimental unit using a non-coated dry commercial feed pellet with a size of 4 mm (EWOS Micro 100). Test diets (TP1, TP2, and NCP) were prepared as follows; the test products, Probiotic 1 or Probiotic 2, were suspended in an oil mix (blend of fish oils) by using Ultraturrax T50 (with a dispersion tool), then feed pellets were coated with the blended mix oil by a Forberg vacuum coater. Temperature in oil mix, non-coated dry feed pellets and during the dispersion and coating process was kept below 40° C. Test diets (TP1, TP2) were prepared to reach a 5% inclusion on feed. Control diet (NCP) was coated with same oil-mix and at same oil inclusion level as TP1 and TP2. Study diets were coated with 18% weight-based oil-mix. The study diets were given from the day of commencement of the feeding and throughout the whole study including the challenge period. To cover all this period, experimental and control feed were prepared on five occasions (Table 46).

TABLE 46
Feed batches prepared of each experimental/control diet.

| | | Amount | | Test product |
|---|---|---|---|---|
| | Pellet | prepared | Feed | Lot. |
| Preparations | size | (Kg) | JNo. | No. |
| FEED #1 | 4 mm | 10 | 2122 | — |
| | | 10 | 2123 | Chinook 3-1 |
| | | 10 | 2124 | Chinook 3-2 |
| FEED #2 | 4 mm | 10 | 2125 | — |
| | | 10 | 2126 | Chinook 4-1 |
| | | 10 | 2127 | Chinook 4-2 |
| FEED #3 | 4 mm | 10 | 2135 | — |
| | | 10 | 2136 | Chinook 4-1 |
| | | 10 | 2137 | Chinook 4-2 |
| FEED #4 | 4 mm | 10 | 2140 | — |
| | | 10 | 2141 | Chinook4-1 |
| | | 10 | 2142 | Chinook 4-2 |
| FEED #5 | 4 mm | 10 | 2143 | — |
| | | 10 | 2144 | Chinook 4-1 |
| | | 10 | 2145 | Chinook 4-2 |

Experimental and control feed were delivered with automatic feeders. Fish were fed in excess (115% of the regular feeding for proper growth) during the experimental feeding period, except for days of feeding reduction/starving before samplings (feeding at 60% of normal ration) or final euthanasia (full starving for 24 hours) of challenge survivors.

During the acclimation and first 28 days of experimental feeding periods, Specific Feeding Ratio (SFR) was 1.54±0.1%. During challenge period, SFR was 1.45±0.1%. Automatic feeders were used with micro-rations delivered from 16:00 to 04:00 on the next day (approx. 96 pulses in 12 h). During the challenge period and after mortalities started, feeding ratio was adjusted daily for each individual study tank.

### Weight and Length Records

All study fish were individually recorded for weight and length on stocking day (study day −21), one day before experimental/control feeding started (study day −1) and one day before cohabitation challenge started (study day 28). Furthermore, a sample of fish (N=20/tank) was registered for weight and length on study day 14. Only the first 28 days of experimental/control feeding were used for growth and performance evaluations. Table 8 shows average population weight per tank on all sampling points.

TABLE 47
Study groups and weight recordings at stocking and during the
growth and performance evaluation period (study days −1
through 90). All weight data presented as Average ± SD (grams).

| | | W on | W on | W on | Weight | |
|---|---|---|---|---|---|---|
| | | day −21 | day −1 | day 14 | on day 28 | Weight |
| Tank | Group | (N = | (N = | (N = | (N = | on day 90 |
| ID | ID | 82) | 82) | 20) | 77) | (N = >38) |
| B103 | NCP | 114.4 ± | 139.2 ± | 176.8 ± | 210.6 ± | 261.8 ± |
| | | 7.8 | 11.8 | 16.7 | 24.3 | 30.5 |
| B106 | NCP | 113.8 ± | 136.6 ± | 174.9 ± | 208.6 ± | 361.0 ± |
| | | 8.7 | 14.1 | 19.9 | 28.3 | 30.4 |
| B101 | TP1 | 114.4 ± | 143.2 ± | 181.9 ± | 212.6 ± | 379.0 ± |
| | | 8.5 | 14.6 | 16.6 | 25.6 | 30.9 |
| B102 | TP1 | 115.3 ± | 141.8 ± | 177.8 ± | 216.7 ± | 370.5 ± |
| | | 8.3 | 13.4 | 24.6 | 23.7 | 31.0 |
| B104 | TP2 | 114.1 ± | 138.4 ± | 176.7 ± | 210.6 ± | 372.9 ± |
| | | 9.6 | 13.9 | 19.2 | 25.5 | 30.8 |
| B105 | TP2 | 114.2 ± | 138.8 ± | 175.0 ± | 211.5 ± | 364.3 ± |
| | | 8.1 | 11.5 | 14.5 | 22.0 | 30.5 |

Before samplings (24 hours) feeding was reduced to a 60% of the corresponding amount. All study fish were sedated with Isoeugenol 50% in the study tanks before weight samplings. Then fish were anaesthetized with Benzocaine 20% for an optimum handling.

### Preparation for Challenge

After the first 28 days of experimental/control feeding a cohabitation challenge against P. salmonis took place. One week before challenge (study day 21) temperature was raised to about 15° C. in order to acclimate all study fish to the disease challenge temperature. From then on, temperature was maintained throughout the study until termination (15.1±0.2° C.).

### Challenge

### Challenge Isolate

The challenge isolate P. salmonis have been kept in an ultra-freezer at −75 to −85° C. The frozen material was thawed and grown on cysteine haemoglobin agar plates, harvested and diluted with Leibovitz-15 medium.

### Challenge Procedure

The challenge was performed by cohabitation with P. salmonis inoculated shedders in sea water. The three study groups were challenged in two replicated tanks each (same tanks as during the first 28 days of experimental/control feeding) according to Table 48. The challenge carriers (shedders; originated from same original batch as test fish and kept on a separate tank under similar water characteristics) were anesthetised, marked using Visible Implant Elastomer (VIE)-tags by injecting a small amount of red VIE tag intradermally (right mandible) and then i.p. injected with P. salmonis using 1 ml disposable syringes with short needless (0.5×8 mm) in accordance with C-1022 and C-1025, before adding them to the challenge tanks. The total amount of shedders was 21.4% of the total amount of test fish in the tanks. Each shedder was i.p. injected with 0.1 ml of the challenge isolate at a concentration of 3.9×10⁶ colony forming units (CFU)/ml. Shedder fish were starved for 24 hours before being inoculated.

TABLE 48
Allocation of fish for the cohabitation challenge.

| | | | | Inoculum | |
|---|---|---|---|---|---|
| Tank | Experimental | Test | Shedder | volume | Challenge |
| ID | Group | fish N | fish N | (ml/fish) | material |
| B103 | NCP | 70 | 14* | 0.1 | P. salmonis |
| B106 | NCP | 70 | 15 | 0.1 | JNo. 2004 |
| B101 | TP1 | 70 | 15 | 0.1 | at 3.9 × 10⁶ |
| B102 | TP1 | 70 | 15 | 0.1 | CFU/ml |
| B104 | TP2 | 69* | 15 | 0.1 | |
| B105 | TP2 | 70 | 15 | 0.1 | |

*One shedder fish died unexpectedly before time on tank B103. Similarly, one test/cohabitant fish died unexpectedly on tank B104 before time.

### Termination

Mortality was observed throughout a 61 days period after challenge started.

### Outcome Parameters—Measures of Effect

Fish growth and survival are the two main outcome parameters in the study. Samples and registration of sampled fish and mortalities, as well as feed stability records are also outcome parameters in the study.

### Statistical Analysis

Data was analysed for descriptive statistics and significant differences among groups by GraphPad Prism 9 (version 9.3.1, GraphPad Software, LLC.). Tanks and/or treatment/groups were used for the analysis of growth performance. Normality of the data was checked with various tests. One-way ANOVA (parametric or non-parametric) with multiple comparisons tests were run depending on the characteristics of the data. Survival analyses between experimental groups was tested under a mantel-Cox test.

### Weight and Length

All study fish were individually recorded for weight and length on stocking day (study day −21), one day before experimental/control feeding started (study day −1) and one day before cohabitation challenge started (study day 28). Furthermore, a sample of fish (N=20/tank) was registered for weight and length on study day 14. All mortalities during challenge as well as all survivors were also registered for these parameters.

### Anatomopathological Examination and IFAT Preparations

100% of challenged fish that died in the P. salmonis challenge as well as all survivors after elimination were examined by necropsy. Furthermore, a head kidney smear was prepared for each mortality and survivor fish into an untreated glass slide for later immunofluorescence antibody test (IFAT) analyses. Smears were fixed with acetone for >3 minutes, let dry at room temperature and then stored at −20° C.

### Bacteriology

Bacteriological analyses were performed. In total, head kidneys from 63 fish were scored for being positive/negative for P. salmonis growth on APS media with 59 out of 63 plates being positive. Furthermore, agar plates with TSA+sal and FMM media were used for bacteriological analyses on kidneys from 62 and 37 fish, respectively.

TABLE 49
Samplings

| Sampling | Study | N of fish | |
|---|---|---|---|
| number | day | sampled | Tissues sampled |
| S1 | −1 | 30 (5/tank) | Blood serum |
| | | | Head kidney |
| S2 | 28 | 30 (5/tank) | Pyloric caeca |
| | | | Distal intestine |
| S3 | 90-91 | 60 (10/tank) | Gut/gut contents |

The study included 3 sampling points that involved tissue samples.

On each sampling (S1, S2 and S3), and one tank at a time, all fish in the tank were sedated with Isoeugenol 50%, and then either 5 (S1 and S2) or 10 (S3) were netted out into a bucket containing Benzocaine 20% as anaesthetic. Fish were then euthanized before proceeding to samples collection. For all fish in all three samplings weight and length were recorded. Sampling tubes/flasks with their contents (RNALater/formalin) as well as acetone and positive-charged-slides were provided.

### Feed Sampling and IVP Stability

Study feeds were sampled on each preparation day. Two samples of >100 grams were taken from each experimental/control diet, labelled and light-proof stored at about 4° C. until delivery. A protocol was used to assess experimental/control feeds stability (i.e., CFU counts). Feed was processed and CFU counts were performed as follows about 10 g feed was weighed and placed into a 50 ml tube. 20 mL sterile PBS was added into the 50 ml tube. The mixture was soaked at least 30 minutes and vortexed well. 4500 μl sterile PBS was poured into four 15 ml centrifuge tubes, and labelled label 1-4 for making serial dilutions. 500 μl of supernatant of the feed-PBS-soaked mixture was added to tube 1 (10⁻¹ dilution), and mixed using a vortex for 10 seconds. Serial dilutions were made until 10⁻⁴, repeating vortex every time. Each dilution (100 μl) was plated in duplicate in Man, Rogosa and Sharpe (MRS) agar plates and spread evenly using a Drigalsky loop. Procedure was repeated with the untreated feed for a negative feed control (this was performed before the experimental feeds to avoid contamination). Plates were incubated at ˜22° C. for 3 to 4 days. After final day of incubation, colonies were counted from pictures taken to the plates using Image software. CFUs were calculated based on colonies per dilution.

### Results

### Observations and Records

Feeding, care and registration of mortalities was carried out daily. All mortalities and survivors were stored at −20° C. in bags labelled per day per tank. Stored fish will be eliminated by incineration after this Final Report has been accepted.

Two fish died during the first days of challenge period due to unexpected causes. One test fish on tank B104 and one shedder fish on tank B103. Both fish were not included for cumulative mortalities values at the end of the challenge.

### Weight and Performance

Weight gain (WG), Specific Growth Rate (SGR) and Thermal Growth Coefficient (TGC) of the test products TP1 and TP2 compared to the control group (NCP) were calculated for the periods between S1 (study day −1—before experimental/control feeding started—) and S2 (study day 28—one day the disease challenge started—) as well as between S1 and S3 (study day 90-final day of challenge-) (Table 50). Comparisons were also made for fish length and condition factor at each point (Table 51).

TABLE 50
Weight and performance parameters for the periods between start
feeding (Day −1), challenge (Day 29) and end of study (Day 90).

| | Weight (g ± SD) | | | Period D −1 to D 28 | | |
|---|---|---|---|---|---|---|
| | D −1 | D 28 | D 90 | SGR | TGC | WG |
| NCP | 137.9 ± | 209.6 ± | 361.4 ± | 1.50 ± | 2.05 ± | 52.0 ± |
| | 1.83 | 1.40 | 0.57 | 0.02 | 0.03 | 1.00 |
| TP1 | 142.5 ± | 214.6 ± | 374.8 ± | 1.46 ± | 2.02 ± | 50.6 ± |
| | 1.00 | 2.90 | 6.04 | 0.07 | 0.10 | 3.09 |
| TP2 | 138.6 ± | 211.0 ± | 368.6 ± | 1.50 ± | 2.06 ± | 52.3 ± |
| | 0.29 | 0.64 | 6.06 | 0.00 | 0.01 | 0.14 |
| | Period D −1 to D 90 | | | Period D 29 to D 90 | | |
| | SGR | TGC | WG | SGR | TGC | WG |
| NCP | 1.07 ± | 1.51 ± | 162.0 ± | 0.89 ± | 1.29 ± | 72.4 ± |
| | 0.01 | 0.01 | 3.06 | 0.01 | 0.01 | 0.87 |
| TP1 | 1.07 ± | 1.52 ± | 162.9 ± | 0.91 ± | 1.32 ± | 74.6 ± |
| | 0.01 | 0.01 | 2.39 | 0.05 | 0.06 | 5.17 |
| TP2 | 1.09 ± | 1.53 ± | 166.0 ± | 0.91 ± | 1.32 ± | 74.7 ± |
| | 0.01 | 0.04 | 4.94 | 0.03 | 0.05 | 3.41 |

TABLE 51
Length and condition factor at study sampling days −1, 28 and 90.

| | Length (cm ± SD) | | | Condition factor | | |
|---|---|---|---|---|---|---|
| | D −1 | D 28 | D 90 | D −1 | D 28 | D 90 |
| NCP | 22.6 ± | 25.6^{a} ± | 30.5 ± | 1.19 ± | 1.25 ± | 1.26 ± |
| | 0.15^{a} | 0.02^{a} | 0.03 | 0.01^{a} | 0.01^{ab} | 0.00 |
| TP1 | 22.9 ± | 25.8^{b} ± | 30.9 ± | 1.18 ± | 1.24 ± | 1.26 ± |
| | 0.01^{b} | 0.02^{b} | 0.08 | 0.01^{ab} | 0.01^{a} | 0.02 |
| TP2 | 22.8 ± | 25.5^{a} ± | 30.7 ± | 1.17 ± | 1.27 ± | 1.26 ± |
| | 0.00^{ab} | 0.06^{a} | 0.22 | 0.00^{b} | 0.01^{b} | 0.00 |

Values are expressed as means ± SD of two replicates. The letter ^{a} and ^{b} (based on post-hoc results) represent significant differences (p < 0.05) among groups.

### Stability Assessment of Test Products in Feed

Five time points of feed preparations (FEED #1 to #5) (Table 46) were prepared accounting for a total of 15 batches of experimental feeds. All of these were tested for stability. On feed preparation day, two samples of about 100 g were collected per control/experimental diet, and stored at 4° C. These samples were used to monitor the stability of probiotics the next day of preparation (day 1 post preparation). Thereafter, the samples were collected directly from the bag stored at the feed storage room (about 18° C.) on days post preparation 14 and 28.

Evaluation records of stability of probiotics in feed were documented in a specific form for this purpose and are shown in Table 52.

TABLE 52
Test products stability assessments.

| | |
|---|---|
| Feed | Calculated concentration (CFU / ml) |

| batch N | Product | Day 1 | Day 14 | Day 28 |
|---|---|---|---|---|
| 1 | NCP | 0.00E+00 | 7.00E+03 | 0.00E+00 |
| | TP1 | 6.86E+07 | 1.94E+07 | 3.70E+06 |
| | TP2 | 7.21E+07 | 4.04E+07 | 2.10E+06 |
| 2 | NCP | 1.50E+02 | 0.00E+00 | 0.00E+00 |
| | TP1 | 9.00E+06 | 6.60E+07 | 3.10E+07 |
| | TP2 | 4.71E+07 | 5.25E+06 | 3.50E+06 |
| 3 | NCP | 0.00E+00 | 0.00E+00 | 0.00E+00 |
| | TP1 | 6.21E+07 | 4.99E+07 | 2.21E+07 |
| | TP2 | 7.90E+06 | 5.80E+06 | 3.25E+06 |
| 4 | NCP | 0.00E+00 | 0.00E+00 | 0.00E+00 |
| | TP1 | 4.75E+07 | 4.10E+07 | 2.43E+07 |
| | TP2 | 5.50E+06 | 2.30E+06 | 2.05E+06 |
| 5 | NCP | 0.00E+00 | 0.00E+00 | 0.00E+00 |
| | TP1 | 4.26E+07 | 3.25E+07 | 2.12E+07 |
| | TP2 | 6.65E+06 | 2.45E+06 | 1.50E+06 |

### Mortalities and Relative Percent Survival (RPS)

Cumulative mortalities per group on all study tanks as well as pooled results are shown on Table 53. RPS values were calculated. Analysis of survival showed that a statistical significant difference was found between NCP and TP1 (p=0.01). Shedder fish reached a 100% mortality on both challenge tanks between days 10 and 23 post challenge with a 98.9% of these dying between days 10 and 16 post challenge.

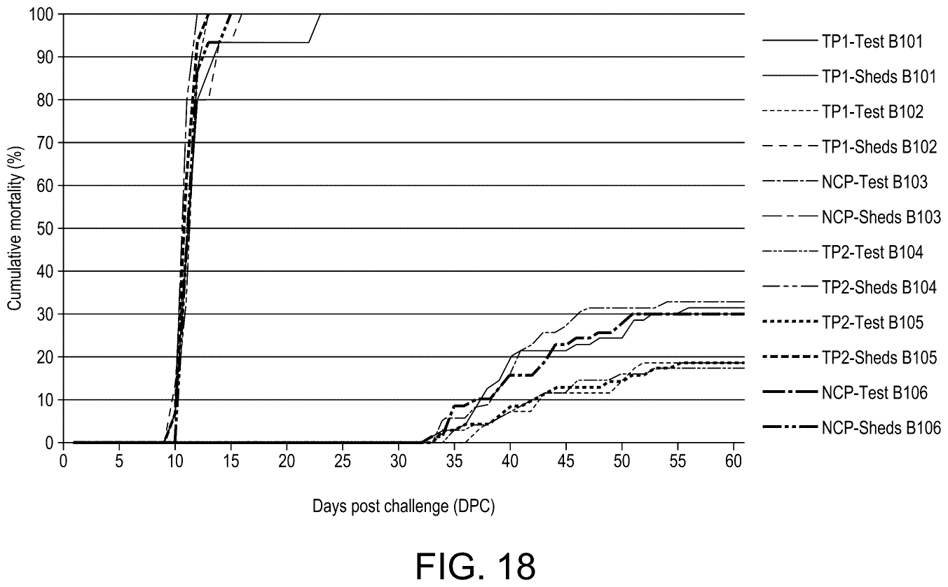

TABLE 53
Challenge tanks B101 to B106 containing all three study groups
NCP, TP1 and TP2 in duplicates. Cumulative mortality and RPS
at the end of the challenge (day 61) on test fish challenged by
cohabitation with P. salmonis injected shedders (FIG. 18).

| | | | | Group | |
|---|---|---|---|---|---|
| | | Cumulative | Cumulative | Cumulative | RPS |
| | | mortality | mortality | mortality | End |
| Group | Tank | (N) | (%) | (av.) | point |
| NCP | B103 | 23 | 32.9 | 31.4 | — |
| | B106 | 21 | 30.0 | | |
| TP1 | B101 | 22 | 31.4 | 25.0 | 20.5 |
| | B102 | 13 | 18.6 | | |
| TP2 | B104 | 12 | 17.4 | 18.0 | 42.8 |
| | B105 | 13 | 18.6 | | |

### Bacterial Load by IFAT and Bacteriology

The IFAT analyses were performed. IFAT results are summarized on Table 54. Bacteriological results for P. salmonis growth on APS media plates are summarized on Table 55. Results for unspecific bacterial growth on other media (TSA+sal and FMM) are not included in table as these were not conclusive.

TABLE 54
Summary of IFAT results. IFAT analyses were performed. Table
shows separate results for fish that died during the challenge
(Mortalities) and fish that survived the challenge (Survivors).
For each tank, the number of fish belonging to the different
IFAT scores are shown. Crosses from one to three depicts
a semi-quantitative system for bacterial load assessment
on the head kidney smears (n = 1 per fish).

| | | Mortalities | | | | Survivors | | | |
|---|---|---|---|---|---|---|---|---|---|
| | | | | | + | | | | + |
| | | | | + | + | | | + | + |
| Group | Tank | Negative | + | + | + | Negative | + | + | + |
| NCP | B103 | 6 | 11 | 5 | 0 | 37 | 1 | 0 | 0 |
| | B106 | 7 | 12 | 1 | 1 | 39 | 0 | 0 | 0 |
| TP1 | B101 | 10 | 11 | 1 | 0 | 36 | 2 | 0 | 0 |
| | B102 | 7 | 5 | 1 | 0 | 42 | 2 | 1 | 2 |
| TP2 | B104 | 2 | 7 | 2 | 1 | 46 | 1 | 0 | 0 |
| | B105 | 4 | 7 | 2 | 0 | 47 | 1 | 0 | 0 |

TABLE 55
Bacteriological analyses; P. salmonis growth on APS media
plates. A total of 63 fish were analysed for bacteriological
analyses by plating head-kidney samples onto TSA media plates.
Incubation was at 22° C. Overall positivity reached a 93.7%.

| | | | | | Positivity |
|---|---|---|---|---|---|
| Group | Tank | Fish N | Positive | Negative | % |
| NCP | B103 | 11 | 10 | 1 | 90.9 |
| | B106 | 12 | 11 | 1 | 91.7 |
| TP1 | B101 | 14 | 14 | 0 | 100 |
| | B102 | 10 | 9 | 1. | 90.0 |
| TP2 | B104 | 7 | 7 | 0 | 100 |
| | B105 | 9 | 8 | 1 | 88.9 |

This disclosed subject matter may be embodied in other forms or carried out in other ways without departing from the spirit or essential characteristics thereof. The present disclosure is therefore to be considered as in all aspects illustrated and not restrictive, the scope of the disclosure being indicated by the appended Claims, and all changes which come within the meaning and range of equivalency are intended to be embraced therein.

Various references are cited throughout this Specification, each of which is incorporated herein by reference in its entirety.

1. A probiotic composition comprising isolated Lactilactobacillus species, said Lactilactobacillus species comprising a first Lactilactobacillus curvatus strain and a second Lactilictobacillus strain, and a carrier suitable for animal administration, wherein said second Lactilactobacillus strain comprises at least one of a second Lactilactobacillus curvatus strain, a Lactilactobacillus sakei strain, a Lactilactobacillus fuchuensis strain and combinations thereof.

2. (canceled)

3. The probiotic composition of claim 1,
(a) wherein said first Lactilactobacillus curvatus strain comprises any of the following strains:
Lactilactobacillus curvatus ELA204093 corresponding to ATCC deposit PTA-127116 or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 1,
Lactilactobacillus curvatus ELA204100 corresponding to ATCC deposit PTA-127117 or a Lactilactobacillus strain having at least having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 2, or
Lactilactobacillus curvatus ELA214388 corresponding to ATCC deposit PTA-127118 or a Lactilactobacillus strain having at least 97% identity, 98% identity, 98.5% identity, 98.6 % identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 3, and combinations thereof; and
wherein said second Lactilactobacillus strain comprises any of the following strains which is different than said first Lactilactobacillus curvatus strain or strains:
Lactilactobacillus curvatus ELA204093 corresponding to ATCC deposit PTA-127116 or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 1,
Lactilactobacillus curvatus ELA204100 corresponding to ATCC deposit PTA-127117 or a Lactilactobacillus strain having at least having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 2,
Lactilactobacillus curvatus ELA214388 corresponding to ATCC deposit PTA-127118 or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 3, or
Lactilactobacillus sakei ELA214391 corresponding to ATCC deposit PTA-127119, and combinations thereof or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 4; or
(b) wherein said first Lactilactobacillus curvatus strain comprises any of the following strains:
Lactilactobacillus curvatus ELA214002 having the genomic sequence of SEQ ID NO: 5, or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 5,
Lactilactobacillus curvatus ELA204023 having the genomic sequence of SEQ ID NO: 6 or a Lactilactobacillus strain having at least having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 6,
Lactilactobacillus curvatus ELA204029 having the genomic sequence of SEQ ID NO: 7 or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 7,
Lactilactobacillus curvatus ELA204033 having the genomic sequence of SEQ ID NO: 8 or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 8, and combinations thereof,
Lactilactobacillus curvatus ELA214059 having the genomic sequence of SEQ ID NO: 9 or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 9,
Lactilactobacillus curvatus ELA214060 having the genomic sequence of SEQ ID NO: 10 or a Lactilactobacillus strain having at least having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 10,
Lactilactobacillus curvatus ELA214061 having the genomic sequence of SEQ ID NO: 11 or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 11,
Lactilactobacillus curvatus ELA214062 having the genomic sequence of SEQ ID NO: 12 or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 12,
Lactilactobacillus curvatus ELA204092 having the genomic sequence of SEQ ID NO: 13 or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 13,
Lactilactobacillus curvatus ELA204096 having the genomic sequence of SEQ ID NO: 14 or a Lactilactobacillus strain having at least having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 14,
Lactilactobacillus curvatus ELA204098 having the genomic sequence of SEQ ID NO: 15 or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 15, or
Lactilactobacillus curvatus ELA214117 having the genomic sequence of SEQ ID NO: 16 or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 16; and
wherein said second Lactilactobacillus strain comprises any of the following strains which is different than said first Lactilactobacillus curvatus strain:
Lactilactobacillus curvatus ELA214002 having the genomic sequence of SEQ ID NO: 5, or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 5,
Lactilactobacillus curvatus ELA204023 having the genomic sequence of SEQ ID NO: 6 or a Lactilactobacillus strain having at least having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 6,
Lactilactobacillus curvatus ELA204029 having the genomic sequence of SEQ ID NO: 7 or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 7,
Lactilactobacillus curvatus ELA204033 having the genomic sequence of SEQ ID NO: 8 or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 8, and combinations thereof,
Lactilactobacillus curvatus ELA214059 having the genomic sequence of SEQ ID NO: 9 or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 9,
Lactilactobacillus curvatus ELA214060 having the genomic sequence of SEQ ID NO: 10 or a Lactilactobacillus strain having at least having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 10,
Lactilactobacillus curvatus ELA214061 having the genomic sequence of SEQ ID NO: 11 or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 11,
Lactilactobacillus curvatus ELA214062 having the genomic sequence of SEQ ID NO: 12 or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 12,
Lactilactobacillus curvatus ELA204092 having the genomic sequence of SEQ ID NO: 13 or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 13,
Lactilactobacillus curvatus ELA204096 having the genomic sequence of SEQ ID NO: 14 or a Lactilactobacillus strain having at least having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 14,
Lactilactobacillus curvatus ELA204098 having the genomic sequence of SEQ ID NO: 15 or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 15,
Lactilactobacillus curvatus ELA214117 having the genomic sequence of SEQ ID NO: 16 or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 16,
Lactilactobacillus fuchuensis ELA214068 having the genomic sequence of SEQ ID NO: 17 or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 17,
Lactilactobacillus sakei ELA214064 having the genomic sequence of SEQ ID NO: 18 or a Lactilactobacillus strain having at least 97% identity, 98% identity, 98.5% identity, 98.6 % identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 18, or
Lactilactobacillus sakei ELA214065 having the genomic sequence of SEQ ID NO: 19, or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 19.

4. (canceled)

5. (canceled)

6. (canceled)

7. (canceled)

8. (canceled)

9. The probiotic composition of claim 1, wherein a ratio of said first Lactilactobacillus curvatus strain and said second Lactilactobacillus strain is 0.75-1.5:1 or 1:0.75-1.5.

10. The probiotic composition of claim 1 comprising isolated Lactilactobacillus species, and a carrier suitable for animal administration, said isolated Lactilactobacillus species comprising at least two of the following:
ELA204093 corresponding to ATCC deposit PTA-127116 or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 1;
ELA204100 corresponding to ATCC deposit PTA-127117 or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 2;
ELA214388 corresponding to ATCC deposit PTA-127118 or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 3; or
ELA214391 corresponding to ATCC deposit PTA-127119 or a Lactilactobacillus strain having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 4.

11. The A probiotic composition of claim 1 comprising a first isolated Lactilactobacillus curvatus strain and a second isolated Lactilactobacillus strain; and a carrier suitable for animal administration;
wherein said composition reduces or inhibits the colonization of an animal by a pathogenic bacterium or provides improved growth performance in an animal when an effective amount is administered to an animal, as compared to an animal not administered the composition; and
(a) wherein the first isolated Lactilactobacillus curvatus strain comprises a nucleic acid sequence having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 1 and wherein the second isolated Lactilactobacillus strain comprises a nucleic acid sequence having at least 97% identity, 98% identity, 98.5% identity, 98.6% identity, 98.7% identity, 98.8 % identity, 98.9% identity, 99% identity, 99.1% identity, 99.2% identity, 99.3% identity, 99.4% identity, or 99.5% identity in genomic sequence to the sequence of SEQ ID NO: 2; or
(b) wherein the first isolated Lactilactobacillus curvatus strain comprises a nucleic acid sequence having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 3 and wherein the second isolated Lactilactobacillus strain comprises a nucleic acid sequence having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 4; or
(c) wherein the first isolated Lactilactobacillus curvatus strain comprises a nucleic acid sequence having at least 97% identity, 98% identity, 98.5% identity, 98.6% identity, 98.7% identity, 98.8% identity, 98.9% identity, 99% identity, 99.1% identity, 99.2% identity, 99.3% 1, and wherein the second isolated Lactilactobacillus strain comprises a nucleic acid sequence having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 3; or
(d) wherein the first isolated Lactilactobacillus curvatus strain comprises a nucleic acid sequence having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 2; and wherein the second isolated Lactilactobacillus strain comprises a nucleic acid sequence having at least 97% identity, 98% identity, 98.5% identity, 98.6% identity, 98.7% identity, 98.8% identity, 98.9% identity, 99% identity, 99.1% identity, 99.2% identity, 99.3% identity, 99.4% identity, or 99.5% identity in genomic sequence to the sequence of SEQ ID NO: 4.

12. (canceled)

13. (canceled)

14. The probiotic composition of claim 1 further comprising at least one Bacillus species wherein the Bacillus species is Bacillus velezensis, Bacillus subtilis, or a combination thereof.

15. (canceled)

16. The probiotic composition of claim 14 wherein the Bacillus species comprises at least one of B. amyloliquefaciens ELA191024, B. subtilis ELA191105, B. amyloliquefaciens ELA202071, and combinations thereof.

17. The probiotic composition of claim 1, wherein said first Lactilactobacillus curvatus strain and said second Lactilactobacillus strain do not have any identifiable antimicrobial resistance genes or only have one antimicrobial resistance gene.

18. (canceled)

19. The probiotic composition of, wherein said first Lactilactobacillus curvatus strain and said second Lactilactobacillus strain do not have any identifiable genes involved in biogenic amines and toxins or only have one gene involved in biogenic amines and toxins.

20. (canceled)

21. The probiotic composition of 1-12, wherein said first Lactilactobacillus curvatus strain and said second Lactilactobacillus strain are sensitive to an antibiotic selected from at least one of Ampicillin, Vancomycin, Gentamicin, Kanamycin, Streptomycin, Erythromycin, Clindamycin, Tetracycline, Chloramphenicol, or combinations thereof.

22. (canceled)

23. The probiotic composition of claim 1, wherein the probiotic composition is in the form of a liquid, dry powder, pellets, suspension, or a combination thereof.

24. The probiotic composition of claim 1, wherein the probiotic composition comprises between about 1×10⁶ and 1×10⁹ CFU/g of said first Lactilactobacillus curvatus strain and said second Lactilactobacillus strain, wherein the probiotic composition comprises at least about 1×10⁶, 1×10⁷ CFU/g, 1×10⁸ CFU/g, or 1×10⁹ CFU/g of said first Lactilactobacillus curvatus strain and said second Lactilactobacillus strain, or comprising from about 1×10⁴ to about 1×10¹⁰ viable spores per gram dry weight of said first Lactilactobacillus curvatus strain and said second Lactilactobacillus strain.

25. (canceled)

26. (canceled)

27. The probiotic composition of claim 1 further comprising a prebiotic, inulin, vitamin D, vitamin C, zinc, N-acetyl-glucosamine, galactooligosaccharides (GOS), lactose, or combinations thereof.

28. (canceled)

29. (canceled)

30. The probiotic composition of claim 1 wherein said first Lactilactobacillus curvatus strain and said second Lactilactobacillus strain are not genetically engineered.

31. (canceled)

32. A direct fed microbial comprising the composition of claim 1 wherein the composition is formulated as animal feed, feed additive, food ingredient, water additive, water-mixed additive, consumable solution, consumable spray additive, consumable solid, consumable gel, injection, or combinations thereof.

33. A fish food product, wherein the fish food product is in the form of pellets, powder, granules, or a combination thereof, comprising the probiotic composition of claim 1, wherein the fish food product comprises about 3-8% w/w of lyophilized said first Lactilactobacillus curvatus strain and said second Lactilactobacillus strain or wherein the fish food product comprises about 0.01-0.2% w/w spray dried spores of said first Lactilactobacillus curvatus strain and said second Lactilactobacillus strain, optionally further comprising food-grade excipients.

34. (canceled)

35. (canceled)

36. (canceled)

37. (canceled)

38. A method for improving feed efficiency in an animal comprising administering to the animal the probiotic composition of claim 1.

39. The method of claim 38 wherein the animal is a fish or is poultry.

40. (canceled)

41. (canceled)

42. (canceled)

43. The method of claim 38 39 wherein the administration is effective in at least one of: improving growth performance, increasing antioxidants, improving immune response, improving survival, and improving performance selected from average daily feed intake (ADFI), average daily gain (ADG) and feed conversion ratio (FCR) as compared to an animal not administered the composition.

44. The method of claim 38 wherein the animal administered the composition exhibits a feed conversion ratio that is decreased by at least 1%, at least 5%, at least 6%, at least 7%, at least 8%, at least 9%, at least 10%, or at least 15%, or exhibits a weight that is increased by at least 1%, at least 5%, at least 10%, at least 15%, at least 25%, or at least 50% as compared to an animal not administered the composition.

45. (canceled)

46. The method of claim 38 wherein the administration improves at least one of humoral immune modulation, bacteriocin production, lymphocyte modulation, and inhibits pathogens and combinations thereof.

47. The method of claim 46 wherein the pathogens are aquatic pathogens and are Aeromonas.

48. (canceled)

49. (canceled)

50. (canceled)

51. (canceled)

52. A method for reducing or inhibiting the colonization of an animal by a pathogenic bacterium or improving disease resistance in an animal comprising administering to an animal an effective amount of the probiotic composition of claim 1.

53. The method of claim 52 wherein the administration is effective in at least one of: improving growth performance, increasing antioxidants, improving immune response and improving survival, as compared to an animal not administered the composition.

54. The method of claim 52 with the proviso that said administration does not comprise administration of an antibiotic.

55. The method of claim 52 wherein said administration is to a salmon and wherein the administration alleviates, or reduces an infection from at least one of Piscirikettsia salmonis and Tanacibaculum maritimum or wherein the administration of the probiotic composition treats, alleviates, or reduces at least one of salmon rickettsial septicemia (SRS) and fit rot.

56. (canceled)

57. (canceled)

58. (canceled)

59. The method of claim 52 wherein the animal is human, non-human, poultry which includes chicken and turkey, bird, cattle, swine, fish, cat, or dog.

60. The method of claim 52 wherein mortality rate is decreased by at least 1%, at least 5%, at least 10%, at least 15%, at least 25%, or at least 50%, or wherein the administration results in said animal exhibiting an improved gut characteristic, as compared to an animal not administered the probiotic composition; wherein said improved gut characteristic includes at least one of: decreasing pathogen-associated lesion formation in the gastrointestinal tract, increasing feed digestibility, increasing meat quality, modulating microbiome, improving short chain fatty acids, and increasing gut health by reducing permeability and inflammation as compared to an animal not administered the probiotic composition.

61. (canceled)

62. The method of claim 60 wherein the pathogen-associated lesion formation in the gastrointestinal tract of the animal administered the probiotic composition is decreased by at least 1%, at least 5%, at least 10%, at least 15%, at least 25%, or at least 50%, as compared to an animal not administered the probiotic composition.
